SEQUENCE LISTING (32 sequences)

SEQ ID NO: 1 - DNA, 2860 bp, Mus musculus
  CDS (104)..(997)
actcggcctctaagctcctggagttgctgcgtccgcgcagtccggaactgcggtgtggccccagccgggacagagagggtccgtcgtaacctgttgagtaactatggaagactatatcaaaatagagaaaattggagaaggtacttacggtgtggtgtataagggtagacacagagtcactggccagatagtggccatgaagaagatcagacttgaaagcgaggaagaaggagtgcccagtactgcaattcgggaaatctctctattaaaagaacttcgacatccaaatatagtcagcctgcaggatgtgctcatgcaggactccaggctgtatctcatctttgagttcctgtccatggacctcaagaagtacctggactccatccctcctgggcagttcatggattcttcactcgttaagagttacttacaccaaatcctccagggaattgtgttttgccactcccggcgagttcttcacagagacttgaaacctcaaaatctattgattgatgacaaaggaacaatcaaactggctgatttcggccttgccagagcgtttggaataccgatacgagtgtacacacacgaggtagtgacgctgtggtaccgatctccagaagtgttgctgggctcggctcgttactccactccggttgacatctggagtatagggaccatatttgcagaactggccaccaagaagccgcttttccacggcgactcagagattgaccagctcttcaggatcttcagagctctgggcactcctaacaacgaagtgtggccagaagtcgagtccctgcaggactacaagaacacctttcccaagtggaagccggggagcctcgcatcccacgtcaagaacctggacgagaacggcttggatttgctctcaaaaatgctagtctatgatcctgccaaacgaatctctggcaaaatggccctgaagcacccgtactttgatgacttggacaatcagattaagaagatgtagccctctggatggatgtccctgtctgctggtcgtaggggaagatcgtgttgtttaccgttggctctcttcctgtcttgtatagttttctttgtttgtaaactgtcatctggacttttcttaatttcctacgtataacttaattaacatgtaaatattattccatatgaatttaaatataattctgtatatgtgcagatgtcactgtggtggctgttaattactataacacaagtgttaattactacaacataagacttgagtctccctagacttcccagcagccattcctgcagctcggagcacagttgaaggagctgagctcaggcctcgtgatgctttcaagtgcctccgtgttctggatatatatgattcctggtcagtttcttgccatttggaaactacaacccacctacggacagtgtttttctacttgtgcttaagcagttgggatgagaaggccaaagacccgaggatgtctagagtaatgacccccagatggaagtgcaccaaagctggctgggtttcacagctagagatcaggggctgtccagagcaggacagcttagaacatttatgaagactccctatttttaggtttgttgtaaagctgttgtctagttggattcctgtgctctgcatggtcagaggtaggttagaggatttgccttggcttctaaatccaatttgaaaactgcttaaaatctcctgtcctctcttagcagtgtctaaaaatgtccttgtccaaatatttagctgagattcctcactttggaaaaggagccgtatcgctgtgctgcttagtgtaattcttagaagcagcctgacttatctgctagcagtcaaagggatgcctgagacgactgctcctcttagaactaaaggctgggatgcttaagtttgtctactgtttggaggatctcggtaagactgagcccctgttcctgtggccacctcagtttaccagtacctcagcctcagcctcctgcatttgctggagtcagggaaggtccccagccctgagccctgacgctcctgattgtagagactgtcagttggaggtaaaacgttcattgaagtagtcagcagccacatgcatttagggcactccagtgtcagagaccatcctggaggtttctaaccctgccgctggcagtctactcccaagacagatcagttagagtggtcagcaaacaccaactgctgcagaaacctgtgttggtgtggttccctgctgctgctgggaactgggcccaagactagagagcttggggctgcggttgatcatgggttctgttcctgcattacaccttgaaatccaagccttctaatatctcccttcggatcataagttgtgaatttggtcctccgcccccgccaggttttctatacttgggtttgtcttgctgacattttcaagagtcctgactaagacggtgattagtgtgacatgacttgagaactaccgatttgaagcacacttgaagttaacaaattctctcatgattatacttttaactttttataagattgcttgagctcacccagatctctgttgggaagtaactgggtaacaaaagccgttgcactggtttgacagctaacaactgttggtactttgtattcagaaggaatgaggtagcgattgaatggctggggtgttgtttccacagtttatacactaaaaatttgggtagccaggaggtagtggcgcgcaatctttaatctcagcagaggcagtcctatctcttgagttggaggccagcctgcctgagttccagaccagccagggctacaggaagaagtcttaaaaaaattttttttccctgtggatgtaaacccatgagaatgactgctgtatctatc

SEQ ID NO: 2 - protein, 297 aa, Mus musculus
MEDYIKIEKIGEGTYGVVYKGRHRVTGQIVAMKKIRLESEEEGVPSTAIREISLLKELRHPNIVSLQDVLMQDSRLYLIFEFLSMDLKKYLDSIPPGQFMDSSLVKSYLHQILQGIVFCHSRRVLHRDLKPQNLLIDDKGTIKLADFGLARAFGIPIRVYTHEVVTLWYRSPEVLLGSARYSTPVDIWSIGTIFAELATKKPLFHGDSEIDQLFRIFRALGTPNNEVWPEVESLQDYKNTFPKWKPGSLASHVKNLDENGLDLLSKMLVYDPAKRISGKMALKHPYFDDLDNQIKKM

SEQ ID NO: 3 - DNA, 1923 bp, Homo sapiens
  CDS (143)..(1036)
agcgcggtgagtttgaaactgctcgcacttggcttcaaagctggctcttggaaattgagcggagagcgacgcggttgttgtagctgccgctgcggccgccgcggaataataagccgggatctaccatacccattgactaactatggaagattataccaaaatagagaaaattggagaaggtacctatggagttgtgtataagggtagacacaaaactacaggtcaagtggtagccatgaaaaaaatcagactagaaagtgaagaggaaggggttcctagtactgcaattcgggaaatttctctattaaaggaacttcgtcatccaaatatagtcagtcttcaggatgtgcttatgcaggattccaggttatatctcatctttgagtttctttccatggatctgaagaaatacttggattctatccctcctggtcagtacatggattcttcacttgttaagagttatttataccaaatcctacaggggattgtgttttgtcactctagaagagttcttcacagagacttaaaacctcaaaatctcttgattgatgacaaaggaacaattaaactggctgattttggccttgccagagcttttggaatacctatcagagtatatacacatgaggtagtaacactctggtacagatctccagaagtattgctggggtcagctcgttactcaactccagttgacatttggagtataggcaccatatttgctgaactagcaactaagaaaccacttttccatggggattcagaaattgatcaactcttcaggattttcagagctttgggcactcccaataatgaagtgtggccagaagtggaatctttacaggactataagaatacatttcccaaatggaaaccaggaagcctagcatcccatgtcaaaaacttggatgaaaatggcttggatttgctctcgaaaatgttaatctatgatccagccaaacgaatttctggcaaaatggcactgaatcatccatattttaatgatttggacaatcagattaagaagatgtagctttctgacaaaaagtttccatatgttatatcaacagatagttgtgtttttattgttaactcttgtctatttttgtcttatatatatttctttgttatcaaacttcagctgtacttcgtcttctaatttcaaaaatataacttaaaaatgtaaatattctatatgaatttaaatataattctgtaaatgtgtgtaggtctcactgtaacaactatttgttactataataaaactataatattgatgtcaggaatcaggaaaaaatttgagttggcttaaatcatctcagtccttatggcagttttattttcctgtagttggaactactaaaatttaggaaaatgctaagttcaagtttcgtaatgctttgaagtatttttatgctctgaatgtttaaatgttctcatcagtttcttgccatgttgttaactatacaacctggctaaagatgaatatttttctactggtattttaatttttgacctaaatgtttaagcattcggaatgagaaaactatacagatttgagaaatgatgctaaatttataggagttttcagtaacttaaaaagctaacatgagagcatgccaaaatttgctaagtcttacaaagatcaagggctgtccgcaacagggaagaacagttttgaaaatttatgaactatcttatttttaggtaggttttgaaagctttttgtctaagtgaattcttatgccttggtcagagtaataactgaaggagttgcttatcttggctttcgagtctgagtttaaaactacacattttgacatagtgtttattagcagccatctaaaaaggctctaatgtatatttaactaaaattactagctttgggaattaaactgtttaacaaataaaaaaaaaaaa

SEQ ID NO: 4 - protein, 297 aa, Homo sapiens
MEDYTKIEKIGEGTYGVVYKGRHKTTGQVVAMKKIRLESEEEGVPSTAIREISLLKELRHPNIVSLQDVLMQDSRLYLIFEFLSMDLKKYLDSIPPGQYMDSSLVKSYLYQILQGIVFCHSRRVLHRDLKPQNLLIDDKGTIKLADFGLARAFGIPIRVYTHEVVTLWYRSPEVLLGSARYSTPVDIWSIGTIFAELATKKPLFHGDSEIDQLFRIFRALGTPNNEVWPEVESLQDYKNTFPKWKPGSLASHVKNLDENGLDLLSKMLIYDPAKRISGKMALNHPYFNDLDNQIKKM

SEQ ID NO: 5 - DNA, 2827 bp, Mus musculus
  CDS (174)..(1442)
ttgaactacaagaccagcagccggcgggcggcgcgcagctcgcggctcctgggctctacgctcctcccgcctgcagcctctgctccgcgggtcggccgccagggtcccgcgctgctccggagcggcgggcgctgtgcacgctctgccgccgacagaaccggaccggagccgcgatgccgggcacctcgaggcattcgggtcgcgatgcgggctcagccctgctctcgctgcatcaggaagaccaagagaatgtcaaccccgaaaaactggcgccagcccagcagccgcgggcgcaggcggtgctgaaggccgggaacgtgcgtggacccgcgccgcagcagaagctcaagactcgacgggttgctcctcttaaggaccttcctataaacgatgagcatgtcactgctggtccttcatggaaagcagtcagtaaacagcctgccttcaccattcatgtggatgaagcagaagagactcagaagaggccagctgagcttaaagaaacagagtgtgaagatgccctggcttttaatgcagctgtctctttacccggagcaagaaaaccactgacacctcttgactatccaatggatggcagttttgaatcaccacatgctatggatatgtctattgtcttagaagacaagccagtgaacgttaatgaagtacctgactatcaagaagacattcacacgtaccttagggaaatggaggttaaatgtaaacctaaagtgggttacatgaagaggcagccagacatcactaacagcatgagggccatccttgtggactggctggttgaggtgggagaagaatataaactacagaatgagaccctgcatttggctgtgaactacattgatagattcctctcctccatgtctgtgttaagagggaagcttcagcttgtaggcacggctgctatgctgctagcttcgaagtttgaagaaatataccccccagaagtagcagagtttgtgtatattacagacgatacctattccaagaagcaggttctgagaatggagcacctagtattgaaagtccttgcttttgacttggctgcaccaacagtaaatcagttccttacccagtacttcctgcacctgcagcctgcaaactgtaaggttgaaagcttagcaatgtttttgggagaactgagtttgatagatgctgacccgtaccttaagtacctgccttcactcattgctggagctgccttccacttggctctctacacagtcacaggacagagctggcctgagtcattggcacaacagactggatataccctggagagtcttaagccttgtcttgtggaccttcaccagacctacctcaaagcgccacaacatgcccaacagtcaatacgggaaaagtacaagcattcaaaatatcacagtgtttctcttctcaacccaccagagacactaagtgtgtgagtgaaagactgccggctttgtttgaaacaggagtcgctcggagtccatgctgtacagtttttatgtcgggttttagtttcacaatcacttctgaatgtagatggtatagccacagacaaattatggtatccattgcttttaaaatggttttaatttgtatatcttttgtacatgtgtctatttggataattttaattggttaacattggtgctgccaactgtcaagatattgagaataaactgatttggaaatctttgcaagtcaaagttaatctgagataaaaatatgaatgatgtcgtttagaaactaacttgatttgttttattgtacagtggggagtaggaaaagattttataaaactaaaatatttcagggaaaaagaccttcaaattctggcattgattgattattctaagcaaccaaaccaaactgctgacttgttcataaataaccaaaagtagacaaactgttttcaatgtagcagaactcattcggctctcagatgttacagtagtttatggagacattttatttatcaactttaacaaataaggtcttacatgagttgggaagcttgtgcaacagaaaagttttccagaaagtttattcaaagcataatgttaaagacaaacagtgtatcaagaagttcagcttgtaagatacccagagctgttagcactgagtatgtatacacacacacacacacagagatccaccactttagcttagttagcagttctcttcttcttcttccttttcccttggcctttacttggtgccttctggtttgcactggtctgagctctgtgaatgaagtaatataacagctcactgtatcccatctgtcctggattgggtcactgggactctgaaaggtgccccattttatagaaccagcttttaatgtatatttgaaagaggccagaaatttgttaccttattttctttgggttcttctctggctccaaaataatcatttcttcttcttcactatctgatagcactataggggcacctttggaaaagtccaaaaaaggagtttactgcttaagcacatcagaggtcctaacgctcccatctccaggctaagggagctcccaagctctactgcccggaggctgacactctttccgaatcttaccctgttctaagtaagtagctccttgcagctctgaaaatttgtaaaaattgatgttttctataaactctttctttcctttttccccagaggacatgtaggccagaatgctctaattcttgctaccaggactgcccatgaaaaatgtagaaaactgctttgtttgcccatttttctcttgaacagtaattatatatgtgtgtaaataaagcatgcaaattaaccctttaaaaaaaaaaaaaaaaa

SEQ ID NO: 6 - protein, 422 aa, Mus musculus
MPGTSRHSGRDAGSALLSLHQEDQENVNPEKLAPAQQPRAQAVLKAGNVRGPAPQQKLKTRRVAPLKDLPINDEHVTAGPSWKAVSKQPAFTIHVDEAEETQKRPAELKETECEDALAFNAAVSLPGARKPLTPLDYPMDGSFESPHAMDMSIVLEDKPVNVNEVPDYQEDIHTYLREMEVKCKPKVGYMKRQPDITNSMRAILVDWLVEVGEEYKLQNETLHLAVNYIDRFLSSMSVLRGKLQLVGTAAMLLASKFEEIYPPEVAEFVYITDDTYSKKQVLRMEHLVLKVLAFDLAAPTVNQFLTQYFLHLQPANCKVESLAMFLGELSLIDADPYLKYLPSLIAGAAFHLALYTVTGQSWPESLAQQTGYTLESLKPCLVDLHQTYLKAPQHAQQSIREKYKHSKYHSVSLLNPPETLSV

SEQ ID NO: 7 - DNA, 2811 bp, Homo sapiens
  CDS (306)..(1604)
ccatttcaatagtcgcgggatacttgaactgcaagaacagccgccgctccggcgggctgctcgctgcatctctgggcgtctttggctcgccacgctgggcagtgcctgcctgcgcctttcgcaacctcctcggccctgcgtggtctcgagctgggtgagcgagcgggcgggctggtaggctggcctgggctgcgaccggcggctacgactattctttggccgggtcggtgcgagtggtcggctgggcagagtgcacgctgcttggcgccgcaggctgatcccgccgtccactcccgggagcagtgatgttgggcaactctgcgccggggcctgcgacccgcgaggcgggctcggcgctgctagcattgcagcagacggcgctccaagaggaccaggagaatatcaacccggaaaaggcagcgcccgtccaacaaccgcggacccgggccgcgctggcggtactgaagtccgggaacccgcggggtctagcgcagcagcagaggccgaagacgagacgggttgcaccccttaaggatcttcctgtaaatgatgagcatgtcaccgttcctccttggaaagcaaacagtaaacagcctgcgttcaccattcatgtggatgaagcagaaaaagaagctcagaagaagccagctgaatctcaaaaaatagagcgtgaagatgccctggcttttaattcagccattagtttacctggacccagaaaaccattggtccctcttgattatccaatggatggtagttttgagtcaccacatactatggacatgtcaattgtattagaagatgaaaagccagtgagtgttaatgaagtaccagactaccatgaggatattcacacataccttagggaaatggaggttaaatgtaaacctaaagtgggttacatgaagaaacagccagacatcactaacagtatgagagctatcctcgtggactggttagttgaagtaggagaagaatataaactacagaatgagaccctgcatttggctgtgaactacattgataggttcctgtcttccatgtcagtgctgagaggaaaacttcagcttgtgggcactgctgctatgctgttagcctcaaagtttgaagaaatataccccccagaagtagcagagtttgtgtacattacagatgatacctacaccaagaaacaagttctgagaatggagcatctagttttgaaagtccttacttttgacttagctgctccaacagtaaatcagtttcttacccaatactttctgcatcagcagcctgcaaactgcaaagttgaaagtttagcaatgtttttgggagaattaagtttgatagatgctgacccatacctcaagtatttgccatcagttattgctggagctgcctttcatttagcactctacacagtcacgggacaaagctggcctgaatcattaatacgaaagactggatataccctggaaagtcttaagccttgtctcatggaccttcaccagacctacctcaaagcaccacagcatgcacaacagtcaataagagaaaagtacaaaaattcaaagtatcatggtgtttctctcctcaacccaccagagacactaaatctgtaacaatgaaagactgcctttgttttctaagatgtaaatcactcaaagtatatggtgtacagtttttaacttaggttttaattttacaatcatttctgaatacagaagttgtggccaagtacaaattatggtatctattactttttaaatggttttaatttgtatatcttttgtatatgtatctgtcttagatatttggctaattttaagtggttttgttaaagtattaatgatgccagctgtcaggataataaattgatttggaaaactttgcaagtcaaatttaacttcttcaggattttgcttagtaaagaagtttacttggtttactatataatgggaagtgaaaagccttcctctaaaattaaagtaggtttaggaaaacagaccctcaaattctgacattcattttcctaagcaactggatcaatttgctgacttgggcataatctaatctaagcatatctgaatacagtattcagagatagatacagtagagattccccagactttttcgctctttgtaaaacctgtttgtttaggttttgcgaggtaaactcaacagaggttgggagtggaagagggtgggaagcttatatgcaaattaacagacgagaaatgctccagaaggtttattattttaaagcacattaaaaacaaaaaactatttttaaaatcctgctagattttataatggatttgtgaataaaaaatacccagggttctcagaatggaataaatatcccttttaatagttatatatacagatatacaactgttagctttaattggcagctctcttcttttttcttcttttcactggctttttacttggtgctttttcttgttttgcactggtggtctgtgttctgtgaataaagcaaagtaagaatttactaagagtatgttaagttttggattattgaaataagaggcatttcttagttttccagtaggatctaaaatgtgtcagctatgagtaagactggcatccaagaagtttatattatagatttaggtcctaatttttataaatcacaaggtaaaaaaatcacagaacagatggatctctaatgaaaaagggatgtctttttgtttatagtcatgtggcaagatgagagtaaaaccagagagcaaacctctataagtgttgagtatatgtatacatttgaaataaaccagaaatttgttaccttaaaaaaaaaaaaaa

SEQ ID NO: 8 - protein, 432 aa, Homo sapiens
MLGNSAPGPATREAGSALLALQQTALQEDQENINPEKAAPVQQPRTRAALAVLKSGNPRGLAQQQRPKTRRVAPLKDLPVNDEHVTVPPWKANSKQPAFTIHVDEAEKEAQKKPAESQKIEREDALAFNSAISLPGPRKPLVPLDYPMDGSFESPHTMDMSIVLEDEKPVSVNEVPDYHEDIHTYLREMEVKCKPKVGYMKKQPDITNSMRAILVDWLVEVGEEYKLQNETLHLAVNYIDRFLSSMSVLRGKLQLVGTAAMLLASKFEEIYPPEVAEFVYITDDTYTKKQVLRMEHLVLKVLTFDLAAPTVNQFLTQYFLHQQPANCKVESLAMFLGELSLIDADPYLKYLPSVIAGAAFHLALYTVTGQSWPESLIRKTGYTLESLKPCLMDLHQTYLKAPQHAQQSIREKYKNSKYHGVSLLNPPETLNL

SEQ ID NO: 9 - DNA, 5148 bp, Mus musculus
  CDS (217)..(2106)
ccacttccctagttacagacaccacttaacagcaggaaacctccacttgctctcctctgccttctctccattgcagccattcccattttcaagactgccagtctggtcctttcaacgttccatgctgcctggtgcccgggagcccaggccttctgtgactccaggacaggaaggtacttctccatacacttcccagaactctgaaggggtgcagacatggcgggaagtgctgtggacagcgccaaccacctgacctacttttttggcaacatcacccgggaagaggctgaagactacctggtccagggaggcatgaccgatgggctctacctgctacgccagagccgcaattacctgggtggttttgctttgtcggtggctcacaacaggaaggcacaccactacaccatcgagagggaacttaatggcacctacgccatctccgggggcagggcccatgccagcccagcagacctctgccattaccactcccaggaacctgatggccttatctgcctccttaagaagcccttcaaccggcccccgggagtacagcccaagaccggaccctttgaggacctgaaggagaacctcatcagggaatatgtgaaacagacctggaaccttcagggccaggctctggagcaagccatcatcagccagaagccccagctggagaagctgatcgccaccacggcccatgagaagatgccctggttccatggcaacatctccagagatgaatcagagcagacggtcctcatagggtcaaagaccaatggaaaattcctgatcagggccagagacaacagcggctcctatgctctgtgcctgctgcacgaagggaaagtattgcactaccgcattgacagggacaagaccgggaagctctccattcctgaggggaagaagtttgacaccctctggcagctagtggaacattactcttacaagccagatgggctactaagagtcctcacggtaccatgccaaaagattggtgcacagatgggccacccaggaagcccaaatgcccatcccgtgacttggtcaccgggtggaataatctcaaggatcaaatcctactccttcccaaagcctggccacaaaaagcctgccccaccccaagggagccgtccagagagcactgtgtccttcaacccctatgagccaacgggagggccctggggcccagacagaggccttcagagagaagccctgcccatggacacagaggtgtacgagagcccttatgctgaccctgaagagatccggcccaaagaggtctacctggacaggagcctgctgaccctggaggacaatgaactgggctccggtaacttcgggactgtgaaaaagggatactaccaaatgaaaaaagttgtgaaaaccgtggctgtgaaaatcctgaagaacgaggccaacgacccggctttgaaggacgagctgctggcagaggcgaacgtcatgcagcagctggacaacccctacattgtgcgcatgatcggaatctgcgaggcggagtcctggatgctggtgatggagatggcggagctggggccgctcaacaagtacctgcagcagaacaggcacattaaggataagaacatcatagagctggttcaccaggtttccatggggatgaagtatttggaagagagcaactttgtgcacagagatctggctgcgcggaacgtgcttctggtcacacagcactatgccaagatcagcgatttcggtctttccaaagccctgcgtgctgatgaaaactactacaaggcccagacccacgggaagtggcccgtgaagtggtacgcccccgaatgcatcaactactacaagttctccagtaagagtgacgtctggagcttcggagtcctgatgtgggaagcgttctcctatgggcagaagccctacagagggatgaaagggagcgaagtgaccgccatgctggagaaaggagagcggatggggtgccctgcaggatgcccgagagagatgtacgacctgatgaacctgtgctggacttacgatgtggagaacaggccaggattcacagctgtggaactgaggcttcgcaattactactacgacgtggttaactaacagctccggcgcctgtccgtgcacaccacggattcccaagcgatcacaggaaattcattcagatgaactggctctcagagtttcatctccctctgcccggagtgagagctaagctaacataggactcaccctcacaacaggtctggtgcccagagacagacaagcagcaagaccttggggcctgtggagtcgtctctctggtctttgtttccatctgtgtggtcttcgctatcggtcacgtttgggagccatttccaaattctcttgacatcattccgttcctctgggtccaggatttcagtgtcccctgaagatcagaaaagaaaagtgctttgccactccagcaaaggacataaagagaaacctcgctggtgcagtcagcaagctggaaggtggttaaggacaatgacaagcctggacactggttttactctcagcgctgagacagagatactgtgtggatgacatgctgagatgtgctggcttggctgacacgcccctggagtccacctccctgcagtcgtgtgtgtttgctggaggaggtgtgaataacgttctgtgtggaggctgccgggggaggtgagctcccatgatcctctgcgactgagcagaagtgtgtcaggcaagggtcagactcccctctctgcagagggaatgcatccaggactccttgttgctgccactaaaaaattgccaaccagcaacttaagggaggaatggctcatttgagctcacagttggaggggataggccatcatggtggaaaagacaacaggcaggaaaggcttggtggcatgaaaaggaagctggaaggtcacatggcatccacattcaggaaacaaagaatgaacaggaagtggggccaggctataaggccctaaaggccacctccggtgacccagttccttcagatccatctatcaaagattccacaggcctctcagacactgaatgccaacagctggggaccaaatgttcaagcacacaaggtgcagtgccaggcaggggccttcctgaatgattacacagtgtggtatcctgacagagcgggtgagccatctcaggtctgtccccttatgagccccgtttaccatcccagattcccccttgggacccccctcatctggatcattcccctccccccaggccctatctccaaacacctaggtactaagtgaacctaggtactaagtcttcaacacatgaattctgaggggacacattcatactggagccgatggcagcctggtgaccaagaccatctcaggatgagtttctgaatggaatcttcattcgccataaaaccagagatgtgaccttccccacaaggaagccttgctaagtgcgacattgagctgaggacacacaagctgagaattccccctctgtcctcctagccttggctaactactgaccatacaccgaatgtcacccattcacggcacttttgctgaggacctaaaagctacaaaggctgcatagccttccatctaggtctagggacatgtacagatgggagcatctcaccttcacctctgagacactgggacattcacagccagggatggtacaggagtccctgctggtggtgggctcaatgaacagagacgatggtgaaatgggtgcctctctttagaccagtggttctcaaccttcctaatgctgagatctttcaatacaggtccttgcgttgtggcgactcccaaccatgaagttattttgttgctacattgtcactgtaattttgctactatgatgaatcgtagtgtagatatctgatatgcaggatatcggatatgtaaatatctggtatgcaggatatgtgaaatgagggcacatcccacaggttgggaaccactgcctttgatagggaggcactatgcttagagcatggttaggaagtatttagaagctattggtgtgtcttggaattgtgttagtaggtcaaccgtcctgtggagggaaggaagggggtggaggaaatccctgtatgttgaattcattgagcagacctgcagaacacagccttggtgaggaagtcccatgggtcagaccacctgtagatttagtcaggagaggaagggccactgtatagttatggagacaggactgtctgctctgagcaagaagttctctgctgagctcctagcctaccttcccttgtccccagggctatagaaaggccacctcgaagaccagggagcatgtgtgagaagtctgtggaggcctctgccttcttcctggcctctctagcagtgcctctccttcccagggatttgggactgaccgttatctggctatgactgagcagagtggagggatgggctttgccccccttcagaaagccgaaaccggttcccatgtgcagagtcacatgtggccatcgaccagggctctcatctggtgaaaactgtgtcttactgtcaagattaattctctcccctgggccactgaggagggcagatggcaagaggatcctccctagaggatttagactatgaatgcccactaaatttgcaaggtcagaaactagccaaggtccttctcaggcatctatccttaacttggtctctccacaagctgcctctgatgctccgagccccttctgcctggcagctgggctccctgtgtgcttttacctatgacccttggtcagcagagtacaaggagcacctaagctgcctcgccacctcccactgactgcaaacaccacatacctcatgcggaaccgaatgtctctccagtaaacaccccagccagccattcataaaaacctgtctctgtgtgcgctgggacatgtcctccctgtaccccgggcctgctttgtgtgccagacagtgactccacagggatgccaggtctgtgattgcattgctccttgcagagggaagccctcggttgtccttctctgggaatgctgccacccagagcagcacacagtgattccctgttatttcagaaattattttaaataaagatctactattagtcttgtgaaaaaaaaaaaaaaaaaaaaaaaaaaaaaaaaaaaaaaaaaaaaaa

SEQ ID NO: 10 - protein, 629 aa, Mus musculus
MAGSAVDSANHLTYFFGNITREEAEDYLVQGGMTDGLYLLRQSRNYLGGFALSVAHNRKAHHYTIERELNGTYAISGGRAHASPADLCHYHSQEPDGLICLLKKPFNRPPGVQPKTGPFEDLKENLIREYVKQTWNLQGQALEQAIISQKPQLEKLIATTAHEKMPWFHGNISRDESEQTVLIGSKTNGKFLIRARDNSGSYALCLLHEGKVLHYRIDRDKTGKLSIPEGKKFDTLWQLVEHYSYKPDGLLRVLTVPCQKIGAQMGHPGSPNAHPVTWSPGGIISRIKSYSFPKPGHKKPAPPQGSRPESTVSFNPYEPTGGPWGPDRGLQREALPMDTEVYESPYADPEEIRPKEVYLDRSLLTLEDNELGSGNFGTVKKGYYQMKKVVKTVAVKILKNEANDPALKDELLAEANVMQQLDNPYIVRMIGICEAESWMLVMEMAELGPLNKYLQQNRHIKDKNIIELVHQVSMGMKYLEESNFVHRDLAARNVLLVTQHYAKISDFGLSKALRADENYYKAQTHGKWPVKWYAPECINYYKFSSKSDVWSFGVLMWEAFSYGQKPYRGMKGSEVTAMLEKGERMGCPAGCPREMYDLMNLCWTYDVENRPGFTAVELRLRNYYYDVVN

SEQ ID NO: 11 - DNA, 5079 bp, Homo sapiens
  CDS (206)..(2113)
acactgggaggaagtgcgggccgcctgcccgggcgcgttaaggaagttgcccaaaatgaggaagagccgcgggcccggcggctgaggccaccccggcggcggctggagagcgaggaggagcgggtggccccgcgctgcgcccgccctcgcctcacctggcgcaggtggacacctgcgcaggtgtgtgccctccggcccctgaagcatggccagcagcggcatggctgacagcgccaaccacctgcccttctttttcggcaacatcacccgggaggaggcagaagattacctggtccaggggggcatgagtgatgggctttatttgctgcgccagagccgcaactacctgggtggcttcgccctgtccgtggcccacgggaggaaggcacaccactacaccatcgagcgggagctgaatggcacctacgccatcgccggtggcaggacccatgccagccccgccgacctctgccactaccactcccaggagtctgatggcctggtctgcctcctcaagaagcccttcaaccggccccaaggggtgcagcccaagactgggccctttgaggatttgaaggaaaacctcatcagggaatatgtgaagcagacatggaacctgcagggtcaggctctggagcaggccatcatcagtcagaagcctcagctggagaagctgatcgctaccacagcccatgaaaaaatgccttggttccatggaaaaatctctcgggaagaatctgagcaaattgtcctgataggatcaaagacaaatggaaagttcctgatccgagccagagacaacaacggctcctacgccctgtgcctgctgcacgaagggaaggtgctgcactatcgcatcgacaaagacaagacagggaagctctccatccccgagggaaagaagttcgacacgctctggcagctagtcgagcattattcttataaagcagatggtttgttaagagttcttactgtcccatgtcaaaaaatcggcacacagggaaatgttaattttggaggccgtccacaacttccaggttcccatcctgcgacttggtcagcgggtggaataatctcaagaatcaaatcatactccttcccaaagcctggccacagaaagtcctcccctgcccaagggaaccggcaagagagtactgtgtcattcaatccgtatgagccagaacttgcaccctgggctgcagacaaaggcccccagagagaagccctacccatggacacagaggtgtacgagagcccctacgcggaccccgaggagatcaggcccaaggaggtttacctggaccgaaagctgctgacgctggaagacaaagaactgggctctggtaattttggaactgtgaaaaagggctactaccaaatgaaaaaagttgtgaaaaccgtggctgtgaaaatactgaaaaacgaggccaatgaccccgctcttaaagatgagttattagcagaagcaaatgtcatgcagcagctggacaacccgtacatcgtgcggatgatcgggatatgcgaggccgagtcctggatgctggttatggagatggcagaacttggtcccctcaataagtatttgcagcagaacagacatgtcaaggataagaacatcatagaactggttcatcaggtttccatgggcatgaagtacttggaggagagcaattttgtgcacagagatctggctgcaagaaatgtgttgctagttacccaacattacgccaagatcagtgatttcggactttccaaagcactgcgtgctgatgaaaactactacaaggcccagacccatggaaagtggcctgtcaagtggtacgctccggaatgcatcaactactacaagttctccagcaaaagcgatgtctggagctttggagtgttgatgtgggaagcattctcctatgggcagaagccatatcgagggatgaaaggaagtgaagtcaccgctatgttagagaaaggagagcggatggggtgccctgcagggtgtccaagagagatgtacgatctcatgaatctgtgctggacatacgatgtggaaaacaggcccggattcgcagcagtggaactgcggctgcgcaattactactatgacgtggtgaactaaccgctcccgcacctgtcggtggctgcctttgatcacaggagcaatcacaggaaaatgtatccagaggaattgattgtcagccacctccctctgccagtcgggagagccaggcttggatggaacatgcccacaacttgtcacccaaagcctgtcccaggactcaccctccacaaagcaaaggcagtcccgggagaaaagacggatggcaggatccaaggggctagctggatttgtttgttttcttgtctgtgtgattttcatacaggttatttttacgatctgtttccaaatccctttcatgtctttccacttctctgggtcccggggtgcatttgttactcatcgggcccagggacattgcagagtggcctagagcactctcaccccaagcggccttttccaaatgcccaaggatgccttagcatgtgactcctgaagggaaggcaaaggcagaggaatttggctgcttctacggccatgagactgatccctggccactgaaaagctttcctgacaataaaaatgttttgaggctttaaaaagaaaatcaagtttgaccagtgcagtttctaagcatgtagccagttaaggaaagaaagaaagaaaaaaaaaaaaggcctggatactgcttttgctgtctctgttatgagatggaagacttacatgtttgtgataaaaggggaccatgagaatgaattggcttggcttactttccccctgaaatcctctctcctgcagactgtcttgaagacctggtgactggtaaataaagccctgcatggaggctgcacagcaggggcaagaggcccatcccccagcatctcactgaggacagcttcaggctgccttcctctgaacgtggtccacaccttcctctcctccacagagagggtgccgccagaatcccctgtcgctttctgtgtctgcaatggggggcagcacagggatcaaagccatctaaagagtttccaaagaaagtattaattcagaacaagccaaagaccctgagcctcaccacaaacaggccttttggagtgtgaatttgagttgaagatacaagatcggagaatgattttctggtcttaactaatcctcatcttcatgtttgatctttaagaagtcatcacccattgatttcagttttgctgtacctcttgaaagttaaagagacatctcagcactttaggaggccgaggcgggtggatcacttgaggtaaggagtttgagactagcctggccaatatggtaaaaccccatctctactaaaaatacaaaaattagccgggcatggtggcatgtgtctgtagtcccagctactcgggaggctgaggcaggagaatcgcttgaacccaggaaacggaggtcgcagtgagccaagatcatgccactgcactccagcttgggcatcacagcgagactctgtcaaaacaaacaaacaaaaaaacaacttaaagaggtaatttagccatcattcttatgccagcagatataaataaacttggacccatctggtcttcagctaaacctgagacattttaaagtgcatggacagccatggacagcaggccctcctctaacaggggatgcaaggcatggagaaagacaatcagtacccaagctcagccacagaagacaggagtcactcatataacttgtgtttagaagtttttggtagccacgcacactttctgaaatcacactatctggtggtttaatcatatttttaaagacagaatccctgagtgctgagcagattctcaaaacacatttagaatccctgaaattagaaagatcaatgacaaaatatctgtcagccaggccacaaacaggtgtaaaattatgaaaggagtggttggatgtgccaagtttggtaaagtggtgactgcatctgagaaagaggctgtgaggctgaactcttggtggcttccttctgtaacttccagagggagtcttcaacacaggccccgtgctcgtaggaatacggtagcacctatgtaggaagtgcgtggagttttctgtcttctttctgtgtgatttttggcctttttatcagcacttctcccctcccaggagcctggggatgccaaacatccagaatgtgatgggacaagatgggggcaggggcctcacctccctgcagaggtccggccaggtctccttgtccctggacaatctcctgagcctctctgcttggtggagcaggcacctgtgtgcagaattcccactgtggccagcacgaggaagtcttttctagtgaaaatgtgtcttgtggtcaggaataattatcctttcccctgtagccaccaaggagggcaaatagagaaaggtaacctaattgaaggattggtcatgtgaaaagggctacatttgggaagctgggaaaggcctccaggcttctagagcagctagcttgggctggattctcatacccaggctgccccttggattgttctacccaagcttttccctggggtctgggctcactccataaggtaaggtgccttttaccttatggtccttctttagcaggtaacaaaggagcatcaggggcaggctgccctggtggcatcacactggctagtgaggccgtgaatatcttgtcccccagcagggccgacagtttctatcacagaaaacagtgtgttcagtggtgaaaatcgttgcatgcatgttttcatctgagcgtgtccttctcccatactccctatcagccagccctgcctgtagctgctgtatggtgattgcacttggacatcagtccaatgactgcaagtcggcctggattttcacttgcagaggctacagctgcattgtcaggtctcccagccctgcagagagctccctccactggttagcagtgtgttgtgttttccattcatttcagaagagctacattgtgtcactggacatttttaaaaactgtgatttttaataaaaatttaaaatttgctttgtgatgaaaaaaa

SEQ ID NO: 12 - protein, 635 aa, Homo sapiens
MASSGMADSANHLPFFFGNITREEAEDYLVQGGMSDGLYLLRQSRNYLGGFALSVAHGRKAHHYTIERELNGTYAIAGGRTHASPADLCHYHSQESDGLVCLLKKPFNRPQGVQPKTGPFEDLKENLIREYVKQTWNLQGQALEQAIISQKPQLEKLIATTAHEKMPWFHGKISREESEQIVLIGSKTNGKFLIRARDNNGSYALCLLHEGKVLHYRIDKDKTGKLSIPEGKKFDTLWQLVEHYSYKADGLLRVLTVPCQKIGTQGNVNFGGRPQLPGSHPATWSAGGIISRIKSYSFPKPGHRKSSPAQGNRQESTVSFNPYEPELAPWAADKGPQREALPMDTEVYESPYADPEEIRPKEVYLDRKLLTLEDKELGSGNFGTVKKGYYQMKKVVKTVAVKILKNEANDPALKDELLAEANVMQQLDNPYIVRMIGICEAESWMLVMEMAELGPLNKYLQQNRHVKDKNIIELVHQVSMGMKYLEESNFVHRDLAARNVLLVTQHYAKISDFGLSKALRADENYYKAQTHGKWPVKWYAPECINYYKFSSKSDVWSFGVLMWEAFSYGQKPYRGMKGSEVTAMLEKGERMGCPAGCPREMYDLMNLCWTYDVENRPGFAAVELRLRNYYYDVVN

SEQ ID NO: 13 - DNA, 683 bp, Mus musculus
  CDS (105)..(365)
ggagggaactgtggtcagggaactgttcgtgggcacagctgcgcagttctgtcagcgcagcgcgatcaccagctcccagcgccgcagcccccagcgcacccaggatgatctcagccgtgatcttgttcttgctccttttggtggaacaagcagccgccctgggagagccgcagctctgctatatcctggatgctgtcctgtttttgtatggtattgtccttaccctactctactgtcgactcaagatccaggtccgaaaggcagctatagccagccgtgagaaagcagatgctgtctacacgggcctgaacacccggagccaggagacatatgagactctgaagcatgagaaaccaccccagtagcttcagaacagacgtgcttggctgcattcttttcccacttctaattctctccgagccctcttggtcacctctgtgctttgaaggttggctgaccttattcacataatgatgctagctaggctctacatcagtgtacactggcaggtccccatctccgttaaagacttactcactgacatttctcttcttccagcctcctttgcttcatttctttttccttccctgatcctcgactctcactaaacaatggaaagggattatcccccaataaagctgccagagacctgactcaaaaaaaaaaaaaaaaaaaaaaaaaaa

SEQ ID NO: 14 - protein, 86 aa, Mus musculus
MISAVILFLLLLVEQAAALGEPQLCYILDAVLFLYGIVLTLLYCRLKIQVRKAAIASREKADAVYTGLNTRSQETYETLKHEKPPQ

SEQ ID NO: 15 - DNA, 591 bp, Homo sapiens
  CDS (26)..(286)
cagaacggccgatctccagcccaagatgattccagcagtggtcttgctcttactccttttggttgaacaagcagcggccctgggagagcctcagctctgctatatcctggatgccatcctgtttctgtatggaattgtcctcaccctcctctactgtcgactgaagatccaagtgcgaaaggcagctataaccagctatgagaaatcagatggtgtttacacgggcctgagcaccaggaaccaggagacttacgagactctgaagcatgagaaaccaccacagtagctttagaatagatgcggtcatattcttctttggcttctggttcttccagccctcatggttggcatcacatatgcctgcatgccattaacaccagctggccctacccctataatgatcctgtgtcctaaattaatatacaccagtggttcctcctccctgttaaagactaatgctcagatgctgtttacggatatttatattctagtctcactctcttgtcccacccttcttctcttccccattcccaactccagctaaaatatgggaagggagaacccccaataaaactgccatggactggactc

SEQ ID NO: 16 - protein, 86 aa, Homo sapiens
MIPAVVLLLLLLVEQAAALGEPQLCYILDAILFLYGIVLTLLYCRLKIQVRKAAITSYEKSDGVYTGLSTRNQETYETLKHEKPPQ

SEQ ID NO: 17 - DNA, 5023 bp, Mus musculus
  CDS (357)..(2900)
gcgcctccggggactcgcggaccgcacagaccggccaggcgcgctgggcatgctcagtcgcggggcgccaggctcgccagtcggaagccagtgctcagcggagccggccggggcgcaccgagagcgcgcggcactcgctgctgcagcggcggccgggccgcggcgcatccgggcggggacccgaggcgagtcccacgcggcgtccccgagccgccggcagcggcgcccgctccagaaaggcaaagcgaaccaggggccatcagcgcgactggcgtcggcggggctctgacgctccgcggcggccacctcagagtcgctgcccgccccggccgggccgggtccgggagccgtcggacatggagccgtggaagcagtgcgctcagtggctcatccacagcaaggtgctgccccccaaccatcgggtgacctgggactcggcgcaggtgttcgacctggcgcagacccttcgcgatggagtcctgctctgccagctgctcaacaacctgcggccacactccatcaatctgaaggagatcaacctgaggccccagatgtcccagtttctctgtttgaagaacatccggacattcctggccgcctgctgcgatacctttggaatgaggaaaagtgaactttttgaggcatttgacctatttgatgttcgagactttggaaaggttatagaaacattatctcggctatcccgaacaccaatagcattggccacaggaatcaggccgttccccactgaagaaagtataaatgatgaagacatctacaaaggccttcccgacttaatagatgaaacccgcgtggaagatgaagaagacctctatgactgtgtgtacggcgaggatgaaggcggagaagtctatgaagacttgatgaaggcggaggaagcccagcaacctaaatcacaagaaaatgatatacggagttgttgcctagcagaaattaggcagacagaagaaaaatacacagaaacgttggagtcaatagaaaaatatttcatggcaccattgaaaagatttctgacggcggcggaatttgattcagtatttattaacattcctgaccttgtaaaagttcatcggagcttaatgcaagagattcatgattccattgtaaataaagatgaccagaacttgtatcaagtttttattaactacaaggaaagattggttatttatgggcagtactgcagtggagtggagtcagccatctctaacctagactacatctctaagacaaaagaagatgtcaaactgaaattagaggaatgttcaaaaagagccaataacgggaagtttactctgcgcgatttgctggtcgttccaatgcagcgcgtgctgaagtatcacctcctccttcaggagctggtaaaacacacccatgaccctatggagaaggcgaacctgaagctggctctggatgcaatgaaggacttggcacaatatgtgaatgaagtaaaaagagataatgagacccttcgtgaaattaagcagtttcagctatctatagagaatttgaaccagccagttttgctctttggacgaccacagggagacggtgaaatccgaataaccactctggacaagcatacaaaacaagaaaggcatatcttcttattcgacttggcagtgattgtctgtaaaaggaaaggcgataactacgagatgaaggaaataatagatctccagcagtacaagatagccaacaaccctaccactgacaaagagaataaaaagtggtcttatggcttctacctcatccatacccaaggacaaaatgggttagaattttattgcaaaacaaaagatttaaagaaaaaatggctggaacagtttgaaatggctctatccaacataaggccagactatgcagactccaatttccatgatttcaagatgcacaccttcactcgggtgacatcctgcagggtctgccagatgctcctgaggggaacattttatcaaggctacttatgttttaagtgtggagcaaaagcacataaagaatgtttgggaagagtagacaattgtggcagagttaattccgttgaacaagggccattcaaaccaccagagaaacggaccaatggacttcggagagcttccagacaggtggacccaggtttgccgaagatgcaggtcattaggaactacactggcaccccggccccagggcttcatgaaggaccaccattgcatatccaggcaggcgacactgttgaacttctgagaggagatgcacacagtgtgttctggcagggacgaaacttagcttcaggagaggttggatttttcccaagtgatgctgtcaaaccctctccatgtgtacctaaaccagtagattattcttgccaaccctggtatgctggcccaatggaaagattgcaggcagaaactgaacttattaacagggtgaatagtacttaccttgtaaggcacaggaccaaagaatctggagaatatgcaattagcattaagtacaataatgaagcaaaacacatcaagattttaacaagagatggcttttttcacattgcagaaaatagaaaatttaaaagcttaatggaacttgtggagtactacaagcaccattctctcaaagaagggttcaggaccttagatacaacgctacagtttccatacaaggagccagaacagccagctggacagaggggtaatagaacaggcaacagcttgttgagtcccaaagtactgggtattgccatcgctcggtacgacttctgtgcaagagatatgcgggagctctccctattgaaaggagacatggtgaagatttacacgaagatgagtgcaaatggctggtggagaggtgaagtaaatggcagggtgggctggtttccatccacatatgtggaagaagatgaataaagtcctatccagtgttgaccctgcactaaaatctcaaagacaacataagcagaagcctgcacggcattgagttaactgaagtgtttaaaaagcagcgttcttggctgttcaacaccctctttttttctcttcctaagtcttaatgctgggatttctaaagatgctggtactgacagattaatggctcgcctagagcagtgcaagaaacagcctgccagtctgtcattgtcaggaaccagggcaaaaccaatggcttttctacccccaaagagtccagcaaacccatcagtttagctttctcttacttcattgggccagacaccatggatccagccattaggaaacgctttttgcatatttactttgttctcacagtttttgatgatacaaagaaacagaagtttaattttgcttttccccagcatgaagagaaagtagagcctgccatctgggaaagatccagcttgctggaaagatccagcatttctcgtcttaaattgttcaacaggtgactcactgcctggcaaacccttttatccccagatgttactcatgataacaaaaaagaggctcatgctcaggtttgcatcataagtgataaattacagagagggttttaattattcatttgatgtcctgattatatttccacacttctttatgaagaagaaaaagagaagcacatgaaaggaggtgtctttgtgttaaagcttcacagctttcgtgatttcgtggagtgttttggaaatgtcgtgcttgacaaagggaatcatggccggccagatcatgcttagttgccaggaatcccagaaaggtagaggtgtttacagagacagatcaagcactgtaatcaatccacaggacatcactgtatagctgcaatagctattaacacaagccttttccttctctctagaaggacccagggcagatttgctgatttacaatctttttttttttttaatagtagcacaaaagagctttattgaggatttgggaagaaaggcagcccgagtccttcatttctttctgagacggatggaagggcacctgatgtccgtgtcctcctgcctgctctgttggtccttctgatgcctttctagctatgtcttaatcagtaggaagatccaccttgcagcatgtttctttctctaagctttcactgtgttccatgaggagtcttcagtatcttcctcctgagatgcatggggtccctagcccaggctgacaactcggctcttccatggtggagtgtttctttggtagcagtagctgtgttctgacaagcttctgaaaatgtttcttcttggggaaaaaaaagatgtatttttatcttacaagtgatctagaagcttctcttgtaccagactttcttggtcacctcttacatgtaagggctgagacaacctgttctctcccatctcacaggagtgccagaacctggaagagaccccatgggggctaggacttctcatttaaatagactcaagacctttacctcagaattatgtaaactgtgattgtgttttagaacaattgagcttttgtatgtgtgtaaatggtcaggaaaatgtattttataaaatgtctatagagtcaacttgtgctggcaaggtctactcttggaatggattcctacctataaggtcttacttgtgtcttggggatgttttgtctgttcatgccagcaagtgttgctatggagagtacagcttcccacagattgtaacactcctgtgtacagtattgtagcatgtattgtttatttcagtcaatagactccctgcctcacaaatggtatgtggtcttcctgttgcatttcattggcctcagagtttcatagcagaggatacagagcctcttttcatgacattaccaatgacaactttgtctacatttaatactttatattggaggttttaaatgccacgatctgtgtagatttctaataccaaagacagacataaatgttttccatagactttgtcttgcctgtctgtagcccttgtgtcatatggtgagttagaatgacattttactttgtaatattttgtaaatatgtcaataaaatagttactacttgcaaaaaaaaaaaaaaaaaaaaaaaaaaaaaaaaaaaaaaa

SEQ ID NO: 18 - protein, 847 aa, Mus musculus
MEPWKQCAQWLIHSKVLPPNHRVTWDSAQVFDLAQTLRDGVLLCQLLNNLRPHSINLKEINLRPQMSQFLCLKNIRTFLAACCDTFGMRKSELFEAFDLFDVRDFGKVIETLSRLSRTPIALATGIRPFPTEESINDEDIYKGLPDLIDETRVEDEEDLYDCVYGEDEGGEVYEDLMKAEEAQQPKSQENDIRSCCLAEIRQTEEKYTETLESIEKYFMAPLKRFLTAAEFDSVFINIPDLVKVHRSLMQEIHDSIVNKDDQNLYQVFINYKERLVIYGQYCSGVESAISNLDYISKTKEDVKLKLEECSKRANNGKFTLRDLLVVPMQRVLKYHLLLQELVKHTHDPMEKANLKLALDAMKDLAQYVNEVKRDNETLREIKQFQLSIENLNQPVLLFGRPQGDGEIRITTLDKHTKQERHIFLFDLAVIVCKRKGDNYEMKEIIDLQQYKIANNPTTDKENKKWSYGFYLIHTQGQNGLEFYCKTKDLKKKWLEQFEMALSNIRPDYADSNFHDFKMHTFTRVTSCRVCQMLLRGTFYQGYLCFKCGAKAHKECLGRVDNCGRVNSVEQGPFKPPEKRTNGLRRASRQVDPGLPKMQVIRNYTGTPAPGLHEGPPLHIQAGDTVELLRGDAHSVFWQGRNLASGEVGFFPSDAVKPSPCVPKPVDYSCQPWYAGPMERLQAETELINRVNSTYLVRHRTKESGEYAISIKYNNEAKHIKILTRDGFFHIAENRKFKSLMELVEYYKHHSLKEGFRTLDTTLQFPYKEPEQPAGQRGNRTGNSLLSPKVLGIAIARYDFCARDMRELSLLKGDMVKIYTKMSANGWWRGEVNGRVGWFPSTYVEEDE

SEQ ID NO: 19 - DNA, 4776 bp, Homo sapiens
  CDS (55)..(2598)
cggccgcatccttgccgcccgccccggcccagccgcgtcccggagccgtcgggcatggagccgtggaagcagtgcgcgcagtggctcatccattgcaaggtgctgcccaccaaccaccgggtgacctgggactcggctcaggtgttcgaccttgcgcagaccctccgcgatggagtcctgctctgccagctgcttaacaacctccgggcgcactccatcaacctgaaggagatcaacctgaggccgcagatgtcccagtttctctgtttgaagaacataaggacatttctcacggcctgttgtgagacgtttggaatgaggaaaagtgaacttttcgaggcatttgacttgtttgatgttcgtgactttggaaaggttatagaaacattatcacgactttctcgaacacctatagcattggccacaggaatcaggcccttcccaacagaagaaagcattaatgatgaagacatctacaaaggccttcctgatttaatagatgaaacccttgtggaagatgaagaagatctctatgactgtgtttatggggaagatgaaggtggagaagtctatgaggacttaatgaaggcagaggaagcacatcagcccaaatgtccagaaaatgatatacgaagttgttgtctagcagaaattaagcagacagaagaaaaatatacagaaactttggagtcaatagaaaagtatttcatggcaccactaaaaagatttctgacagcagcagaatttgattcagtattcatcaacattcctgaacttgtaaaacttcatcggaacctaatgcaagagattcatgattccattgtaaataaaaatgaccagaacttgtaccaagtttttattaactacaaggaaagattggttatttacgggcagtactgcagtggagtggagtcagccatctctagtttagactacatttctaagacaaaagaagatgtcaaactgaaattagaggaatgttccaaaagagcaaataatgggaaatttactcttcgagacttgcttgtggttcctatgcaacgtgttttaaagtaccaccttctcctccaggaactggtcaaacataccactgatccgactgagaaggcaaatctgaaactggctcttgatgccatgaaggacttggcacaatatgtgaatgaagtgaaaagagataatgagacccttcgtgaaattaaacagtttcagctatctatagagaatttgaaccaaccagttttgctttttggacgacctcagggagatggtgaaattcgaataaccactctagacaagcataccaaacaagaaaggcatatcttcttatttgatttggcagtgatcgtatgtaagagaaaaggtgataactatgaaatgaaggaaataatagatcttcagcagtacaagatagccaataatcctacaaccgataaagaaaacaaaaagtggtcttatggcttctacctcatccatacccaaggacaaaatgggttagaattttattgcaaaacaaaagatttaaagaagaaatggctagaacagtttgaaatggctttgtctaacataagaccagactatgcagactccaatttccacgacttcaagatgcataccttcactcgagtcacatcctgcaaagtctgccagatgctcctgaggggaacattttatcaaggctatttatgttttaagtgtggagcgagagcacacaaagaatgtttgggaagagtagacaattgtggcagagttaattctggtgaacaagggacactcaaactaccagagaaacggaccaatggactgcgaagaactcctaaacaggtggatccaggtttaccaaagatgcaggtcattaggaactattctggaacaccacccccagctctgcatgaaggaccccctttacagctccaggccggggataccgttgaacttctgaaaggagatgcacacagtctgttttggcagggcagaaatttagcatctggagaggttggattttttccaagtgatgcagtcaagccttgcccatgtgtgcccaaaccagtagattattcttgccaaccctggtatgctggagcaatggaaagattgcaagcagagaccgaacttattaatagggtaaatagtacttaccttgtgaggcacaggaccaaagagtcaggagaatatgcaattagcattaagtacaataatgaagcaaagcacatcaagattttaacaagagatggcttttttcacattgcagaaaatagaaaatttaaaagtttaatggaacttgtggagtactacaagcatcattctctcaaggaagggttcagaaccttagatacaactctgcagtttccatacaaggagccagaacattcagctggacagaggggtaatagagcaggcaacagcttgttaagtccaaaagtgctgggcattgccatcgctcggtatgacttctgtgcaagagatatgagagagttgtccttgttgaaaggagatgtggtgaagatttacacaaagatgagtgcaaatggctggtggagaggagaagtaaatggcagggtgggctggtttccatccacatatgtggaagaggatgaataaattcaaatcccgtgttgcaccctgcaccaaaaatttcagagaagggataaatagaagcctgcacagcatcgtgaattaactgaagtgtttaaaaagctgcatttctggctgttcaacatcctccctccttagcccctcctaagtcttaatgctgagatttctaaagatgctggtactgacagattaatggcttgcctagagctgtgcaagaaacagcctgccagtctgtcattgtcagggaccagggcaaaaccaagagctgttcttcccagaagagccctgcaaacacattggttcgtgcttccctttacttcttctggtcagataccatgaatgccagtcatcagtaaatcttaatacacttttgctttattctcacatgccattcaccagattatttgatggtacaaagaagcagaagtgtaattttccttttcccagcatgacgaaaaattggagttctgccatttgagcagcttactggaaagatccagccttacttgtcttaaattgtccaacaaggtgactcattgcccggcaaacacttttaccctcagatgttactcatgatattataaaatatgaggccagtgctcaggtttgcatcataagtgagctatccctgaagggttttaattacttatttggtgtcctgattatatttgcaaacttctttataaaaggtgaaaaaagcacacaaaagagagggtgtcttcatattaaaccttcacaaccttcatgatttcataggattattttggaaatatagcacttgactttatgaaaggatctgggctaggtatattaagggtagttgccaataacctgaagaagctggcattgtttacagaaacagatcaagggctataatttatgtcattttatagcagcagtatctattaatacatgccttttcctcccatccacctcccccgcacacacacaaagatgacctgggacatgatttttttattcccacattttcttggagcacaaacaactttgttgaggattttggaaggaaagcacaactgggtcctttattcatttctgggacagaaagagggtcagtggacttttgtgggcctccagcttctctcagagtctccccctctgcagcccatcctgggagtgtattaactggagggaagatgggtcttgcagtacatttgttttgcccagccatcactcttttttgtgaggagcctaaatacattcttcctggggtccagagtccccattcaaggcagtcaagttaagacactaacttggccctttcctgatggaaatatttcctccatagcagaagttgtgttctgacaagactgagagagttacatgttgggaaaaaaaagaagcattaacttagtagaactgaaccaggagcattaagttctgaaattttgaatcatctctgaaatgaagcaggtgtagcctgccctctcatcaatccgtctgggtgccagaactcaaggttcagtggacacatccccctgttagagaccctcatgggctaggacttttcatctaggatagattcaagacctttacctcagaattatgtaaactgtgattgtgttttagaaaaattattatttgctaaaaccatttaagtctttgtatatgtgtaaatgatcacaaaaatgtattttataaaatgttctgtacaataaagttacacctcaaagtgtactcttggaatggattctttcctgtaaagtcttatctgcgactctgtctcgggaatgttttgtctgttgccgtcagccgaactttgttatggagggagcagcctcacacaagcagaaacactcctgtggatggtattgtagcatgtattgtttattttagtcaatagaccctctccttataaatggtgtttagtcttcctgttgcatttcatgggcctgggggtttcctagcagaggatattggagcccctttttgtgacattaccaattacatctttgtccacgtttaatactttgttttggaaaatttaaatgctgcagatttgtgtagagttctaataccaaagacagaagtaaatgttttccatatactttgtcttgcctgtatgcagccctcgtgtaatacggtgaattagagtggtatttcactttgtattattttgtaaatatgtcaatataataaatagtgactaaattgataaaaaaa

SEQ ID NO: 20 - protein, 847 aa, Homo sapiens
MEPWKQCAQWLIHCKVLPTNHRVTWDSAQVFDLAQTLRDGVLLCQLLNNLRAHSINLKEINLRPQMSQFLCLKNIRTFLTACCETFGMRKSELFEAFDLFDVRDFGKVIETLSRLSRTPIALATGIRPFPTEESINDEDIYKGLPDLIDETLVEDEEDLYDCVYGEDEGGEVYEDLMKAEEAHQPKCPENDIRSCCLAEIKQTEEKYTETLESIEKYFMAPLKRFLTAAEFDSVFINIPELVKLHRNLMQEIHDSIVNKNDQNLYQVFINYKERLVIYGQYCSGVESAISSLDYISKTKEDVKLKLEECSKRANNGKFTLRDLLVVPMQRVLKYHLLLQELVKHTTDPTEKANLKLALDAMKDLAQYVNEVKRDNETLREIKQFQLSIENLNQPVLLFGRPQGDGEIRITTLDKHTKQERHIFLFDLAVIVCKRKGDNYEMKEIIDLQQYKIANNPTTDKENKKWSYGFYLIHTQGQNGLEFYCKTKDLKKKWLEQFEMALSNIRPDYADSNFHDFKMHTFTRVTSCKVCQMLLRGTFYQGYLCFKCGARAHKECLGRVDNCGRVNSGEQGTLKLPEKRTNGLRRTPKQVDPGLPKMQVIRNYSGTPPPALHEGPPLQLQAGDTVELLKGDAHSLFWQGRNLASGEVGFFPSDAVKPCPCVPKPVDYSCQPWYAGAMERLQAETELINRVNSTYLVRHRTKESGEYAISIKYNNEAKHIKILTRDGFFHIAENRKFKSLMELVEYYKHHSLKEGFRTLDTTLQFPYKEPEHSAGQRGNRAGNSLLSPKVLGIAIARYDFCARDMRELSLLKGDVVKIYTKMSANGWWRGEVNGRVGWFPSTYVEEDE

SEQ ID NO: 21 - DNA, 5654 bp, Mus musculus
  CDS (320)..(2662)
ggggggcggccattctcttctgtccaagagagcagggcaggagtgcaggggcagtagcgaaagcaggctggtgtgtctttaaacttccgttggctgcttagtcacagccccctcgctttgggtgtgtccttcgcgcgctccctccctcttaggtcactcactctttcaaagcctggaataaaaaccacagccaacttccgaagcggtctcattgcccagcagcccccagccagtgacaggttccattcaccctcgttgcccttctccccacgacccttttccagaggcgactagatccctccgtttcatccagcacgccatggacaagttttggtggcacacagcttggggactttgcctcttgcagttgagcctggcacatccacatcagcagatcgatttgaatgtaacctgccgctacgcaggtgtattccatgtggagaaaaatggccgctacagtatctcccggactgaggcagctgacctctgccaggctttcaacagtaccttacccaccatggaccaaatgaagttggccctgagcaagggttttgaaacatgcaggtatgggttcatagaaggaaatgtggtaattccgaggattcatcccaacgctatctgtgcagccaaccacacaggagtatatatcctcgtcacgtccaacacctcccactatgacacatattgcttcaatgcctcagcccctcctgaagaagactgtacatcagtcacagacctacccaattccttcgatggaccggttaccataactattgtcaaccgtgatggtactcgctacagcaagaagggcgagtatagaacacaccaagaagacatcgatgcttcaaacattatagatgacgatgtcagcagcggctccaccatcgagaagagcaccccagaaagctacattttgcacacctaccttcctactgaacagcctactggagatcaggatgactccttctttatccggagcaccttggccaccattgcctcaactgtgcactcaaaaagccatgcagcagctcagaaacaaaataattggatatggtcttggtttggtaattcacagtccacaactcagactcaggagcccacaacgagtgcaactacagccttgatgaccacccctgaaacaccacccaagaggcaagaagcccagaactggttttcatggttatttcaaccatcagagtccaagagtcatcttcatacaacaacgaagatgcctggtacggagtcaaataccaacccaacaggctgggagccaaatgaggaaaatgaagatgaaacagacaaataccccagtttttctggatcaggcattgatgatgatgaagattttatctccagtaccattgcaagtactccacgggtttctgcccgcacagaagacaaccaggactggacccagtggaagccaaaccattcaaacccagaagtactactccagacaaccaccaggatggctgatatagacagaatcagcaccagtgctcatggagaaaattggaccccggaaccacagcctcctttcaataaccatgagtatcaggatgaagaggagaccccacatgctacaagcacaactcctaatagtacagcagaagcagcagctacccagcaggagacgtggtttcagaacggatggcaggggaagaacccccctaccccaagtgaagactcccatgtgacagaagggacaactgcttcggcccacaacaaccatccaagtcaaagaataacaacacagagtcaagaggatgtttcctggacagatttcttcgacccaatctcacatccaatgggacaaggtcatcaaacagaaagcaaggatacagactccagtcatagtacaacccttcagcctactgcagctccaaatacccatttggtggaagacttgaacaggacaggaccactttcagtgacaactccacagagtcattctcagaacttctctacattacatggagagccggaagaagacgaaaaccatccaacaacttctattctgccatctagcactaagagcggcgctaaagatgcaagaagaggtggaagtcttcccacagatacaactacttctgtagaaggctacacctttcaatacccagacacaatggaaaatgggactctcttcccagtgacccctgctaaaactgaggtctttggagaaactgaagtgactcttgctactgactccaacgttaatgttgatggctccttaccaggagatcgagactcatccaaggactccagggggagttcccgcactgtgactcatggatccgaattagctggacactcaagtgcgaaccaggacagtggagtgaccacaacttctggtcctatgaggagacctcagattccagaatggctcatcatcttggcatctctcctggcactggctctgattcttgccgtctgcatcgcggtcaatagtaggagaaggtgtgggcagaagaaaaagctggtgatcaacggtggcaatgggacggtggaagacaggaaacccagtgagctcaacggggaggccagcaagtctcaggaaatggtgcatttggtgaacaaggaaccatcagagaccccggaccagtgtatgacagctgacgagacccggaatctgcagagtgtggacatgaagattggggtgtagtgcctacgccattaacttgaaaagacagcacgattggagacgtcattcctgggagctgggaccctccacagatgcaatgtgctactgactattattattggggattatgtttagcataaaattttctattccttttttattttatgtcatttttttagagtctgttccaatttatgaaaatagcattgctttctgaaatgagggcccaaataatactccattaagaatttgacttttccagttccttcctaggggccttgcattaccagggtatgctaccataggcttctaccaaatgcacattcttggtccctattgaaccttatccctacccaaagtcccagggaacatccaccagctaaggattttccccagaacctagattggtctctgggaggaaatttgaatgggtccatattgcctctccagcagtccaatccttaggcattgctttccagcggggtgggagatcaggtgtactggttacatactctctttatagactcccttctgctggaaaattttcagatgcttctgagagatccccaaagggtgatgctatttatctttagtaagctatttatctttgtttttgaaatatcaaaccctggaggtcctttgttcagtatgacttttttttttattttgttttattttgttttgttttgttttgtttttttaggttactttatcagaagcataacagggtataaattgattcataataagtaccggtccatcttccatcttgacccgttgtgctgtgatccttcagtttctaaacaagcaaagtctgagtctttgtagcacatcagtgtggccttagtatggtcctcctaagctcatgctagagcatctgagcctgctgggttcacccagctcaatctcattgaagatcagggacaggagcactgttttcattccaggctctcaaaggggtttctctcctgttcaagaatctgaagtgagagtaggcaagcttcagtcccttttgtgctttggtgaccacccttttctatttcttaaagaacacattaagtttttatatcttacaacgtttgtggtcctgtgtcatagagaatgatcttattagtgatttcacaaaacattgtggtggggacttgtggcgccttctcataaaaggagaagccaacagaaatgaaagtgtaaacatccagcagtagcttgccacgaggaggaggtgtgatgtacaacccccatctcactctagccatggctgtgtttgttggtgctttcacaagcgagttggtacttagaatcagttccctggggaacccctcaaaaagccacaggactgcccaccactgggatcttatcatcagataagcaggtttatggtttagcaaaaggagaatgctgttaccctcatagttttcacatattgggcaagtgttcattcatctgccaggatgtcccattgctcctggatcttcctaggtatcttgtagaatatattaactctataaaataaggctgtctctaaaatggaacttcctttctaagggtcccacttttactgttgactaaatttatatgtttagtaggtttttttttccaataaaaacaaacaaaaaaaaaaaaaggagaaaaaattttcacctgagtcaggtttgagtttatatcaaaagtggatgtgaagatgttccttaaaggagatacacggactttctggcacaattttgagcctcagctctccttactcatattctagcctccctccttccctgcccatctctcccccagagccatggctttcatggactttctctttctctgtgaatactataagccagttccatatttagacacctggttttcttgttcaaatagacactagatgattcttggctattctccactggtattagaatgtcccctgtccttagagctatcctggcctaccacacccacaacaggctccctggggctctttaacctatatgcagcaagccactctgggattggtcataaaagggctctgtcaagctctccatgaccccttgatgccctttgagggcacaagaagagtcatacaagactcagagctaaagataattctctacccattctaaaaaagtccactagttgggttgtggtaggacattgcttgccatagtggtgatgattctggtagcacaatctgactttgagttactccatgattttgtaaagtattaggagcccttaaagtgttcttttgccatcaactataaatttctcatatatcctggaggctttccttctctttgccttgggtaaaaatcaaatgaggtgaaagataccttcatgtccatattttgtatgttctagctagataaaatggatgtttcagaaaagcctttaaaaatctttatattgacatccataacaccaaaaactgtctttttagctaaaatcgacccaagactgtcacagcaaagtagaagaaacagccatcttctttggaaaagtaaaatgttcataagaacagggattgcccactatgatgataatgatgtactgtgtctttgtgtaagtcctatgtgttgacttttcaaatatagccaattgcttaggtatgtacaatgagtttttctattgttcctttgatttttcagagaccccccctttctttttccagtttttgtatatttattgattgactaataataatgaggaaaacctgatgtgtacattacccgattgaaagtgtgtattggaaaatattaaaaggttgtaaaaaatggaacctagggtctctggtgtgaagggtctgggaagtggagaggggcagaaagtgattgtctggatgtctgaataatatggaagaagtcagaggcagctggcatcctttgttttgttttgttttgttttgttcttttatagcccccccctcctatttttctttggtgtttttgttttgtttgtttgatttttgaaacaggatctcatagagctcagactggtaataaattcaatatgtagccgaaaa

SEQ ID NO: 22 - protein, 780 aa, Mus musculus
MDKFWWHTAWGLCLLQLSLAHPHQQIDLNVTCRYAGVFHVEKNGRYSISRTEAADLCQAFNSTLPTMDQMKLALSKGFETCRYGFIEGNVVIPRIHPNAICAANHTGVYILVTSNTSHYDTYCFNASAPPEEDCTSVTDLPNSFDGPVTITIVNRDGTRYSKKGEYRTHQEDIDASNIIDDDVSSGSTIEKSTPESYILHTYLPTEQPTGDQDDSFFIRSTLATIASTVHSKSHAAAQKQNNWIWSWFGNSQSTTQTQEPTTSATTALMTTPETPPKRQEAQNWFSWLFQPSESKSHLHTTTKMPGTESNTNPTGWEPNEENEDETDKYPSFSGSGIDDDEDFISSTIASTPRVSARTEDNQDWTQWKPNHSNPEVLLQTTTRMADIDRISTSAHGENWTPEPQPPFNNHEYQDEEETPHATSTTPNSTAEAAATQQETWFQNGWQGKNPPTPSEDSHVTEGTTASAHNNHPSQRITTQSQEDVSWTDFFDPISHPMGQGHQTESKDTDSSHSTTLQPTAAPNTHLVEDLNRTGPLSVTTPQSHSQNFSTLHGEPEEDENHPTTSILPSSTKSGAKDARRGGSLPTDTTTSVEGYTFQYPDTMENGTLFPVTPAKTEVFGETEVTLATDSNVNVDGSLPGDRDSSKDSRGSSRTVTHGSELAGHSSANQDSGVTTTSGPMRRPQIPEWLIILASLLALALILAVCIAVNSRRRCGQKKKLVINGGNGTVEDRKPSELNGEASKSQEMVHLVNKEPSETPDQCMTADETRNLQSVDMKIGV

SEQ ID NO: 23 - DNA, 5748 bp, Homo sapiens
  CDS (435)..(2663)
gagaagaaagccagtgcgtctctgggcgcaggggccagtggggctcggaggcacaggcaccccgcgacactccaggttccccgacccacgtccctggcagccccgattatttacagcctcagcagagcacggggcgggggcagaggggcccgcccgggagggctgctacttcttaaaacctctgcgggctgcttagtcacagccccccttgcttgggtgtgtccttcgctcgctccctccctccgtcttaggtcactgttttcaacctcgaataaaaactgcagccaacttccgaggcagcctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccactttgatgagcactagtgctacagcaactgagacagcaaccaagaggcaagaaacctgggattggttttcatggttgtttctaccatcagagtcaaagaatcatcttcacacaacaacacaaatggctggtacgtcttcaaataccatctcagcaggctgggagccaaatgaagaaaatgaagatgaaagagacagacacctcagtttttctggatcaggcattgatgatgatgaagattttatctccagcaccatttcaaccacaccacgggcttttgaccacacaaaacagaaccaggactggacccagtggaacccaagccattcaaatccggaagtgctacttcagacaaccacaaggatgactgatgtagacagaaatggcaccactgcttatgaaggaaactggaacccagaagcacaccctcccctcattcaccatgagcatcatgaggaagaagagaccccacattctacaagcacaatccaggcaactcctagtagtacaacggaagaaacagctacccagaaggaacagtggtttggcaacagatggcatgagggatatcgccaaacacccaaagaagactcccattcgacaacagggacagctgcagcctcagctcataccagccatccaatgcaaggaaggacaacaccaagcccagaggacagttcctggactgatttcttcaacccaatctcacaccccatgggacgaggtcatcaagcaggaagaaggatggatatggactccagtcatagtataacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacatcaagcaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaacagaaaaaaaaaaaaaaaaa

SEQ ID NO: 24 - protein, 742 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 25 - DNA, 3185 bp, Mus musculus
  CDS (7)..(2199)
ctcaccatgagtccctggcagcccctgctcctggctctcctggctttcggctgcagctctgctgccccttaccagcgccagccgacttttgtggtcttccccaaagacctgaaaacctccaacctcacggacacccagctggcagaggcatacttgtaccgctatggttacacccgggccgcccagatgatgggagagaagcagtctctacggccggctttgctgatgcttcagaagcagctctccctgccccagactggtgagctggacagccagacactaaaggccattcgaacaccacgctgtggtgtcccagacgtgggtcgattccaaaccttcaaaggcctcaagtgggaccatcataacatcacatactggatccaaaactactctgaagacttgccgcgagacatgatcgatgacgccttcgcgcgcgccttcgcggtgtggggcgaggtggcacccctcaccttcacccgcgtgtacggacccgaagcggacattgtcatccagtttggtgtcgcggagcacggagacgggtatcccttcgacggcaaggacggccttctggcacacgcctttccccctggcgccggcgttcagggagatgcccatttcgacgacgacgagttgtggtcgctgggcaaaggcgtcgtgatccccacttactatggaaactcaaatggtgccccatgtcactttcccttcaccttcgagggacgctcctattcggcctgcaccacagacggccgcaacgacggcacgccttggtgtagcacaacagctgactacgataaggacggcaaatttggtttctgccctagtgagagactctacacggagcacggcaacggagaaggcaaaccctgtgtgttcccgttcatctttgagggccgctcctactctgcctgcaccactaaaggccgctcggatggttaccgctggtgcgccaccacagccaactatgaccaggataaactgtatggcttctgccctacccgagtggacgcgaccgtagttgggggcaactcggcaggagagctgtgcgtcttccccttcgtcttcctgggcaagcagtactcttcctgtaccagcgacggccgcagggatgggcgcctctggtgtgcgaccacatcgaacttcgacactgacaagaagtggggtttctgtccagaccaagggtacagcctgttcctggtggcagcgcacgagttcggccatgcactgggcttagatcattccagcgtgccggaagcgctcatgtacccgctgtatagctacctcgagggcttccctctgaataaagacgacatagacggcatccagtatctgtatggtcgtggctctaagcctgacccaaggcctccagccaccaccacaactgaaccacagccgacagcacctcccactatgtgtcccactatacctcccacggcctatcccacagtgggccccacggttggccctacaggcgccccctcacctggccccacaagcagcccgtcacctggccctacaggcgccccctcacctggccctacagcgccccctactgcgggctcttctgaggcctctacagagtctttgagtccggcagacaatccttgcaatgtggatgtttttgatgctattgctgagatccagggcgctctgcatttcttcaaggacggttggtactggaagttcctgaatcatagaggaagcccattacagggccccttccttactgcccgcacgtggccagccctgcctgcaacgctggactccgcctttgaggatccgcagaccaagagggttttcttcttctctggacgtcaaatgtgggtgtacacaggcaagaccgtgctgggccccaggagtctggataagttgggtctaggcccagaggtaacccacgtcagcgggcttctcccgcgtcgtctcgggaaggctctgctgttcagcaaggggcgtgtctggagattcgacttgaagtctcagaaggtggatccccagagcgtcattcgcgtggataaggagttctctggtgtgccctggaactcacacgacatcttccagtaccaagacaaagcctatttctgccatggcaaattcttctggcgtgtgagtttccaaaatgaggtgaacaaggtggaccatgaggtgaaccaggtggacgacgtgggctacgtgacctacgacctcctgcagtgcccttgaactagggctccttctttgcttcaaccgtgcagtgcaagtctctagagaccaccaccaccaccaccacacacaaaccccatccgagggaaaggtgctagctggccaggtacagactggtgatctcttctagagactgggaaggagtggaggcaggcagggctctctctgcccaccgtcctttcttgttggactgtttctaataaacacggatccccaaccttttccagctactttagtcaatcagcttatctgtagttgcagatgcatccgagcaagaagacaactttgtagggtggattctgaccttttatttttgtgtggcgtctgagaattgaatcagctggcttttgtgacaggcacttcaccggctaaaccacctctcccgactccagcccttttatttattatgtatgaggttatgttcacatgcatgtatttaacccacagaatgcttactgtgtgtcgggcgcggctccaaccgctgcataaatattaaggtattcagttgcccctactggaaggtattatgtaactatttctctcttacattggagaacaccaccgagctatccactcatcaaacatttattgagagcatccctagggagccaggctctctactgggcgttagggacagaaatgttggttcttccttcaaggattgctcagagattctccgtgtcctgtaaatctgctgaaaccagaccccagactcctctctctcccgagagtccaactcactcactgtggttgctggcagctgcagcatgcgtatacagcatgtgtgctagagaggtagagggggtctgtgcgttatggttcaggtcagactgtgtcctccaggtgagatgacccctcagctggaactgatccaggaaggataaccaagtgtcttcctggcagtcttttttaaataaatgaataaatgaatatttacttaaaaaaaaaaaaaaaaaaaaaaaaaaaaaaaaaaaaaaaaaaaaaaa

SEQ ID NO: 26 - protein, 730 aa, Mus musculus
MSPWQPLLLALLAFGCSSAAPYQRQPTFVVFPKDLKTSNLTDTQLAEAYLYRYGYTRAAQMMGEKQSLRPALLMLQKQLSLPQTGELDSQTLKAIRTPRCGVPDVGRFQTFKGLKWDHHNITYWIQNYSEDLPRDMIDDAFARAFAVWGEVAPLTFTRVYGPEADIVIQFGVAEHGDGYPFDGKDGLLAHAFPPGAGVQGDAHFDDDELWSLGKGVVIPTYYGNSNGAPCHFPFTFEGRSYSACTTDGRNDGTPWCSTTADYDKDGKFGFCPSERLYTEHGNGEGKPCVFPFIFEGRSYSACTTKGRSDGYRWCATTANYDQDKLYGFCPTRVDATVVGGNSAGELCVFPFVFLGKQYSSCTSDGRRDGRLWCATTSNFDTDKKWGFCPDQGYSLFLVAAHEFGHALGLDHSSVPEALMYPLYSYLEGFPLNKDDIDGIQYLYGRGSKPDPRPPATTTTEPQPTAPPTMCPTIPPTAYPTVGPTVGPTGAPSPGPTSSPSPGPTGAPSPGPTAPPTAGSSEASTESLSPADNPCNVDVFDAIAEIQGALHFFKDGWYWKFLNHRGSPLQGPFLTARTWPALPATLDSAFEDPQTKRVFFFSGRQMWVYTGKTVLGPRSLDKLGLGPEVTHVSGLLPRRLGKALLFSKGRVWRFDLKSQKVDPQSVIRVDKEFSGVPWNSHDIFQYQDKAYFCHGKFFWRVSFQNEVNKVDHEVNQVDDVGYVTYDLLQCP

SEQ ID NO: 27 - DNA, 2387 bp, Homo sapiens
  CDS (20)..(2143)
agacacctctgccctcaccatgagcctctggcagcccctggtcctggtgctcctggtgctgggctgctgctttgctgcccccagacagcgccagtccacccttgtgctcttccctggagacctgagaaccaatctcaccgacaggcagctggcagaggaatacctgtaccgctatggttacactcgggtggcagagatgcgtggagagtcgaaatctctggggcctgcgctgctgcttctccagaagcaactgtccctgcccgagaccggtgagctggatagcgccacgctgaaggccatgcgaaccccacggtgcggggtcccagacctgggcagattccaaacctttgagggcgacctcaagtggcaccaccacaacatcacctattggatccaaaactactcggaagacttgccgcgggcggtgattgacgacgcctttgcccgcgccttcgcactgtggagcgcggtgacgccgctcaccttcactcgcgtgtacagccgggacgcagacatcgtcatccagtttggtgtcgcggagcacggagacgggtatcccttcgacgggaaggacgggctcctggcacacgcctttcctcctggccccggcattcagggagacgcccatttcgacgatgacgagttgtggtccctgggcaagggcgtcgtggttccaactcggtttggaaacgcagatggcgcggcctgccacttccccttcatcttcgagggccgctcctactctgcctgcaccaccgacggtcgctccgacggcttgccctggtgcagtaccacggccaactacgacaccgacgaccggtttggcttctgccccagcgagagactctacacccaggacggcaatgctgatgggaaaccctgccagtttccattcatcttccaaggccaatcctactccgcctgcaccacggacggtcgctccgacggctaccgctggtgcgccaccaccgccaactacgaccgggacaagctcttcggcttctgcccgacccgagctgactcgacggtgatggggggcaactcggcgggggagctgtgcgtcttccccttcactttcctgggtaaggagtactcgacctgtaccagcgagggccgcggagatgggcgcctctggtgcgctaccacctcgaactttgacagcgacaagaagtggggcttctgcccggaccaaggatacagtttgttcctcgtggcggcgcatgagttcggccacgcgctgggcttagatcattcctcagtgccggaggcgctcatgtaccctatgtaccgcttcactgaggggccccccttgcataaggacgacgtgaatggcatccggcacctctatggtcctcgccctgaacctgagccacggcctccaaccaccaccacaccgcagcccacggctcccccgacggtctgccccaccggaccccccactgtccacccctcagagcgccccacagctggccccacaggtcccccctcagctggccccacaggtccccccactgctggcccttctacggccactactgtgcctttgagtccggtggacgatgcctgcaacgtgaacatcttcgacgccatcgcggagattgggaaccagctgtatttgttcaaggatgggaagtactggcgattctctgagggcagggggagccggccgcagggccccttccttatcgccgacaagtggcccgcgctgccccgcaagctggactcggtctttgaggagcggctctccaagaagcttttcttcttctctgggcgccaggtgtgggtgtacacaggcgcgtcggtgctgggcccgaggcgtctggacaagctgggcctgggagccgacgtggcccaggtgaccggggccctccggagtggcagggggaagatgctgctgttcagcgggcggcgcctctggaggttcgacgtgaaggcgcagatggtggatccccggagcgccagcgaggtggaccggatgttccccggggtgcctttggacacgcacgacgtcttccagtaccgagagaaagcctatttctgccaggaccgcttctactggcgcgtgagttcccggagtgagttgaaccaggtggaccaagtgggctacgtgacctatgacatcctgcagtgccctgaggactagggctcccgtcctgctttggcagtgccatgtaaatccccactgggaccaaccctggggaaggagccagtttgccggatacaaactggtattctgttctggaggaaagggaggagtggaggtgggctgggccctctcttctcacctttgttttttgttggagtgtttctaataaacttggattctctaacctttaaaaaaaaaaaaaaaaaaaaaaaaaaaaaaaaaaaaaaaaaaaaaaaaaaaa

SEQ ID NO: 28 - protein, 707 aa, Homo sapiens
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTQDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEERLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 29 - DNA, 4682 bp, Mus musculus
  CDS (104)..(3568)
tgtttttacccctccctcctggccttggtggcttcctcaaagtgtctgtggtgcctgaaataccacagttcacacttctttcagggccttgttcctttaagtcatgactcttaaagctcttctggtcacagccctagccttgtgtcatggcttcaatctggacactgaacatcccatgaccttccaagagaatgcaaaaggctttggacagagtgtggtccagcttggcggaaccagtgtggttgttgcagccccccaggaggcaaaggctgttaaccagacaggtgccctctaccagtgtgactacagcacaagccggtgtcaccccatccccctgcaagtacctccagaggctgtgaatatgtccttgggcctgtccctggctgtttctactgtcccccagcagctgctggcctgtggccccacggtgcaccaaaactgcaaggagaatacttatgtgaatggattgtgctatttgttcggctccaacctgctgaggccgccccagcagttcccagaggctctcagagaatgtcctcagcaggagagtgacattgtcttcttgattgatggctccggtagcatcaacaacattgactttcagaagatgaaggagtttgtctcaactgtgatggagcagttcaaaaagtctaaaaccttgttctctttgatgcagtactcggacgagttccggattcacttcaccttcaatgacttcaagagaaaccctagcccaagatcacatgtgagccccataaagcagctgaatgggaggacaaaaactgcctcagggatccggaaagtagtgagagaactgtttcacaaaaccaatggggcccgggagaatgctgcgaagatcctagttgtcatcacagatggagaaaaattcggtgatcccttggattataaggatgtcatccccgaggcagacagagcaggggtcattcgctacgtaattggggtgggaaatgccttcaacaaaccacagtcccgcagagagctcgacaccatcgcatctaagccagctggtgaacacgtgttccaagtggacaactttgaagccctgaataccattcagaaccagcttcaggaaaagatctttgcaattgagggcacgcagacaggaagtaccagctcctttgagcatgagatgtctcaagaaggcttcagtgcttccattacctctaatggtcccttgctgggctctgtggggagctttgactgggcaggtggagccttcctgtatacatcgaaggataaagtcaccttcatcaacacaaccagagtggattcagacatgaatgatgcttacctgggttatgcttctgcagtcatcttgaggaaccgtgtccaaagcttggttttaggagcacctcggtatcagcatattggcttggtggtgatgttcagggagaatttcggtacctgggagccccacactagcatcaagggcagccagattggctcttattttggggcctccctttgctctgtggacatggacgctgatggcaataccaacttgatcctcattggggcccctcattactatgagaagacccgaggaggccaggtgtcagtgtgtcccttgcctcgagggcagagggcacggtggcagtgtgaagctcttctccacggtgatcagggtcatccctggggtcgctttggggcagccctgacagtgctgggagacgtgaatggggacaaactgacagatgtggccattggggccccaggagagcaggagaatcagggcgctgtctacattttttatggagcatcaatagccagcctcagtgcctcccacagccagcggatcataggcgcccacttctcccctgggctccagtacttcgggcagtctctgagtgggggtaaggatctcacaatggatggcttgatggacctggctgttggggcgcaggggcatcttctgctgctcagagcccagccggtgctgagactggaggcaaccatggaattcagccccaagaaagtagcaaggagtgtgtttgcatgtcaagaacaagtattaaaaaacaaggatgctggggaggtcagagtctgcctccgtgtccgcaagaacaccaaggaccgtctgcgcgaaggagatatccagagcactgtcacttatgacctggctttagaccctggccgctcacgtatccgtgccttctttgatgagacaaagaacaacacacgcaggcgcacccaggtctttggattgatgcagaaatgtgaaacactgaagctaattttaccggactgcgtggacgactcagtgagccccatcatcctgcgcctcaattatacactggttggggagcccttgaggtcctttgggaacctccgaccagttctggctatggatgctcagaggttcttcacagctatgtttccctttgagaaaaattgcggcaatgacagcatctgccaagacgatctcagcatcaccatgagtgccatgggcttggacactttggtggtgggaggcccccaggactttaacatgagtgtgactctgagaaatgacggtgaggattcctacgggacccaggttaccgtctactacccatctggcttatcttaccggaaggattcagcaagccagaacccgctcaccaagaagccttggtttgtaaagcctgctgagtccagctcttcttctgaagggcatggggctctgaagagtaccacctggaacataaaccatcccatctttcctgctaattctgaggtcacatttaatgtcacatttgatgtggactctcatgcctcctttgggaacaaactgctcctcaaagccattgtggccagtgagaacaacatgtccaggacccacaaaaccaagtttcagttggagctgcctgtgaagtacgccatctacatgattgtcaccagtgatgagagttctatcagatatctcaacttcacggcttcagagatgaccagtaaggtcatacagcatcagtaccagttcaacaacctgggccagaggagcctccctgtcagtgtagtcttctggatccctgttcagatcaacaatgtgaccgtatgggatcatccccaggtcatcttctcccagaacctctcaagtgcctgtcacactgagcagaaatccccccctcactccaatttccgggatcagcttgaaaggaccccagtgctgaactgctctgttgcagtctgtaagagaattcagtgtgaccttccatccttcaacactcaggaaatattcaatgtcaccctcaagggcaacctatcatttgactggtacatcaagacttctcatggtcacctcctgcttgtgagcagcactgagatcctgtttaatgactctgcgtttgccctgcttccagggcaggagtcgtatgtgaggtctaagacagagaccaaagtggagccatatgaagttcacaatcctgtaccactcattgtgggcagctccattgggggcctggtgctcttggctctcatcactgctggcctatacaagcttggctttttcaagcggcagtacaaggacatgatgaatgaagctgccccccaagacgccccacctcagtaacggccttttctccatgcattgacctccccagcaacaagcaggtctagatggtcagaccaacaccgaagctgatggacacatatgttgttgctgtgatgtcttatacaaaaatggaatgtgtgtgtgtgtgtgtgtgtgagagagagagagagagagaaagagagagagagaaagagagagggagagggagagagagagagagagagagagagaagaaaagaggaagcaagctctgtgtgtggctcacacatttcttgcctgtgaccaatgccttaaatgcatcctcttggaaggaaaggcatctcttgggtttcctagtgtgttagggaagctgctaatgggtccttctctgggcaaggctgtgggacccttgtgggtaagcatagctaaatgctgtgctgtcctctctgaaggaagtaatatttccttgtttgtgtgagctgcagacttgcaagggtttaggtgctgagaatacaagagaagttggaatagagaactggaaagccagcagagatgttcagtgaaaaatgagacgcctcctaccatctagtcatttattattttaacattaatttaaataattgaatcgattaacccgtcaaagtacaaaaaccaaaagatactcaagaatggtaaaaggcagggggtggggaggatgcagctcacagcacagtgctcacctggaatatgcaaggccttgggctcagcatgcgacagtgaaaaccaaggcaaagaaaaaacaaaccaaaccaaaaatgtaagtggaaagactcccttcctagctgttgggggtctctccttagggatattaaagggtatatgtttagaatctattccacaaagattgtaggtacctatatacacacacacacacacacacacatatacacacactgctttaaacaagtggtaacacatttttttactattatgaatgttgtctttttattaatatgctcaaataaatttatataagttataaagtagctcagagaattaccctgtgattgtaatgtatttaacatttaatgtagtctcctttggatatataatttccagccatttgctattatgtacaatgctgaattaataaattgggtatatgtaaatttta

SEQ ID NO: 30 - protein, 1154 aa, Mus musculus
MTLKALLVTALALCHGFNLDTEHPMTFQENAKGFGQSVVQLGGTSVVVAAPQEAKAVNQTGALYQCDYSTSRCHPIPLQVPPEAVNMSLGLSLAVSTVPQQLLACGPTVHQNCKENTYVNGLCYLFGSNLLRPPQQFPEALRECPQQESDIVFLIDGSGSINNIDFQKMKEFVSTVMEQFKKSKTLFSLMQYSDEFRIHFTFNDFKRNPSPRSHVSPIKQLNGRTKTASGIRKVVRELFHKTNGARENAAKILVVITDGEKFGDPLDYKDVIPEADRAGVIRYVIGVGNAFNKPQSRRELDTIASKPAGEHVFQVDNFEALNTIQNQLQEKIFAIEGTQTGSTSSFEHEMSQEGFSASITSNGPLLGSVGSFDWAGGAFLYTSKDKVTFINTTRVDSDMNDAYLGYASAVILRNRVQSLVLGAPRYQHIGLVVMFRENFGTWEPHTSIKGSQIGSYFGASLCSVDMDADGNTNLILIGAPHYYEKTRGGQVSVCPLPRGQRARWQCEALLHGDQGHPWGRFGAALTVLGDVNGDKLTDVAIGAPGEQENQGAVYIFYGASIASLSASHSQRIIGAHFSPGLQYFGQSLSGGKDLTMDGLMDLAVGAQGHLLLLRAQPVLRLEATMEFSPKKVARSVFACQEQVLKNKDAGEVRVCLRVRKNTKDRLREGDIQSTVTYDLALDPGRSRIRAFFDETKNNTRRRTQVFGLMQKCETLKLILPDCVDDSVSPIILRLNYTLVGEPLRSFGNLRPVLAMDAQRFFTAMFPFEKNCGNDSICQDDLSITMSAMGLDTLVVGGPQDFNMSVTLRNDGEDSYGTQVTVYYPSGLSYRKDSASQNPLTKKPWFVKPAESSSSSEGHGALKSTTWNINHPIFPANSEVTFNVTFDVDSHASFGNKLLLKAIVASENNMSRTHKTKFQLELPVKYAIYMIVTSDESSIRYLNFTASEMTSKVIQHQYQFNNLGQRSLPVSVVFWIPVQINNVTVWDHPQVIFSQNLSSACHTEQKSPPHSNFRDQLERTPVLNCSVAVCKRIQCDLPSFNTQEIFNVTLKGNLSFDWYIKTSHGHLLLVSSTEILFNDSAFALLPGQESYVRSKTETKVEPYEVHNPVPLIVGSSIGGLVLLALITAGLYKLGFFKRQYKDMMNEAAPQDAPPQ

SEQ ID NO: 31 - DNA, 4745 bp, Homo sapiens
  CDS (99)..(3560)
ttttctgcccttctttgctttggtggcttccttgtggttcctcagtggtgcctgcaacccctggttcacctccttccaggttctggctccttccagccatggctctcagagtccttctgttaacagccttgaccttatgtcatgggttcaacttggacactgaaaacgcaatgaccttccaagagaacgcaaggggcttcgggcagagcgtggtccagcttcagggatccagggtggtggttggagccccccaggagatagtggctgccaaccaaaggggcagcctctaccagtgcgactacagcacaggctcatgcgagcccatccgcctgcaggtccccgtggaggccgtgaacatgtccctgggcctgtccctggcagccaccaccagcccccctcagctgctggcctgtggtcccaccgtgcaccagacttgcagtgagaacacgtatgtgaaagggctctgcttcctgtttggatccaacctacggcagcagccccagaagttcccagaggccctccgagggtgtcctcaagaggatagtgacattgccttcttgattgatggctctggtagcatcatcccacatgactttcggcggatgaaggagtttgtctcaactgtgatggagcaattaaaaaagtccaaaaccttgttctctttgatgcagtactctgaagaattccggattcactttaccttcaaagagttccagaacaaccctaacccaagatcactggtgaagccaataacgcagctgcttgggcggacacacacggccacgggcatccgcaaagtggtacgagagctgtttaacatcaccaacggagcccgaaagaatgcctttaagatcctagttgtcatcacggatggagaaaagtttggcgatcccttgggatatgaggatgtcatccctgaggcagacagagagggagtcattcgctacgtcattggggtgggagatgccttccgcagtgagaaatcccgccaagagcttaataccatcgcatccaagccgcctcgtgatcacgtgttccaggtgaataactttgaggctctgaagaccattcagaaccagcttcgggagaagatctttgcgatcgagggtactcagacaggaagtagcagctcctttgagcatgagatgtctcaggaaggcttcagcgctgccatcacctctaatggccccttgctgagcactgtggggagctatgactgggctggtggagtctttctatatacatcaaaggagaaaagcaccttcatcaacatgaccagagtggattcagacatgaatgatgcttacttgggttatgctgccgccatcatcttacggaaccgggtgcaaagcctggttctgggggcacctcgatatcagcacatcggcctggtagcgatgttcaggcagaacactggcatgtgggagtccaacgctaatgtcaagggcacccagatcggcgcctacttcggggcctccctctgctccgtggacgtggacagcaacggcagcaccgacctggtcctcatcggggccccccattactacgagcagacccgagggggccaggtgtccgtgtgccccttgcccagggggcagagggctcggtggcagtgtgatgctgttctctacggggagcagggccaaccctggggccgctttggggcagccctaacagtgctgggggacgtaaatggggacaagctgacggacgtggccattggggccccaggagaggaggacaaccggggtgctgtttacctgtttcacggaacctcaggatctggcatcagcccctcccatagccagcggatagcaggctccaagctctctcccaggctccagtattttggtcagtcactgagtgggggccaggacctcacaatggatggactggtagacctgactgtaggagcccaggggcacgtgctgctgctcaggtcccagccagtactgagagtcaaggcaatcatggagttcaatcccagggaagtggcaaggaatgtatttgagtgtaatgatcaggtggtgaaaggcaaggaagccggagaggtcagagtctgcctccatgtccagaagagcacacgggatcggctaagagaaggacagatccagagtgttgtgacttatgacctggctctggactccggccgcccacattcccgcgccgtcttcaatgagacaaagaacagcacacgcagacagacacaggtcttggggctgacccagacttgtgagaccctgaaactacagttgccgaattgcatcgaggacccagtgagccccattgtgctgcgcctgaacttctctctggtgggaacgccattgtctgctttcgggaacctccggccagtgctggcggaggatgctcagagactcttcacagccttgtttccctttgagaagaattgtggcaatgacaacatctgccaggatgacctcagcatcaccttcagtttcatgagcctggactgcctcgtggtgggtgggccccgggagttcaacgtgacagtgactgtgagaaatgatggtgaggactcctacaggacacaggtcaccttcttcttcccgcttgacctgtcctaccggaaggtgtccacgctccagaaccagcgctcacagcgatcctggcgcctggcctgtgagtctgcctcctccaccgaagtgtctggggccttgaagagcaccagctgcagcataaaccaccccatcttcccggaaaactcagaggtcacctttaatatcacgtttgatgtagactctaaggcttcccttggaaacaaactgctcctcaaggccaatgtgaccagtgagaacaacatgcccagaaccaacaaaaccgaattccaactggagctgccggtgaaatatgctgtctacatggtggtcaccagccatggggtctccactaaatatctcaacttcacggcctcagagaataccagtcgggtcatgcagcatcaatatcaggtcagcaacctggggcagaggagcctccccatcagcctggtgttcttggtgcccgtccggctgaaccagactgtcatatgggaccgcccccaggtcaccttctccgagaacctctcgagtacgtgccacaccaaggagcgcttgccctctcactccgactttctggctgagcttcggaaggcccccgtggtgaactgctccatcgctgtctgccagagaatccagtgtgacatcccgttctttggcatccaggaagaattcaatgctaccctcaaaggcaacctctcgtttgactggtacatcaagacctcgcataaccacctcctgatcgtgagcacagctgagatcttgtttaacgattccgtgttcaccctgctgccgggacagggggcgtttgtgaggtcccagacggagaccaaagtggagccgttcgaggtccccaaccccctgccgctcatcgtgggcagctctgtcgggggactgctgctcctggccctcatcaccgccgcgctgtacaagctcggcttcttcaagcggcaatacaaggacatgatgagtgaagggggtcccccgggggccgaaccccagtagcggctccttcccgacagagctgcctctcggtggccagcaggactctgcccagaccacacgtagcccccaggctgctggacacgtcggacagcgaagtatccccgacaggacgggcttgggcttccatttgtgtgtgtgcaagtgtgtatgtgcgtgtgtgcaagtgtctgtgtgcaagtgtgtgcacatgtgtgcgtgtgcgtgcatgtgcacttgcacgcccatgtgtgagtgtgtgcaagtatgtgagtgtgtccaagtgtgtgtgcgtgtgtccatgtgtgtgcaagtgtgtgcatgtgtgcgagtgtgtgcatgtgtgtgctcaggggcgtgtggctcacgtgtgtgactcagatgtctctggcgtgtgggtaggtgacggcagcgtagcctctccggcagaagggaactgcctgggctcccttgtgcgtgggtgaagccgctgctgggttttcctccgggagaggggacggtcaatcctgtgggtgaagacagagggaaacacagcagcttctctccactgaaagaagtgggacttcccgtcgcctgcgagcctgcggcctgctggagcctgcgcagcttggatggagactccatgagaagccgtgggtggaaccaggaacctcctccacaccagcgctgatgcccaataaagatgcccactgaggaatgatgaagcttcctttctggattcatttattatttcaatgtgactttaattttttggatggataagcttgtctatggtacaaaaatcacaaggcattcaagtgtacagtgaaaagtctccctttccagatattcaagtcacctccttaaaggtagtcaagattgtgttttgaggtttccttcagacagattccaggcgatgtgcaagtgtatgcacgtgtgcacacacaccacacatacacacacacaagcttttttacacaaatggtagcatactttatattggtctgtatcttgctttttttcaccaatatttctcagacatcggttcatattaagacataaattactttttcattcttttataccgctgcatagtattccattgtgtgagtgtaccataatgtatttaaccagtcttcttttgatatactattttcattctcttgttattgcatcaatgctgagttaataaatcaaatatatgtcatttttgcatatatgtaaggataa

SEQ ID NO: 32 - protein, 1153 aa, Homo sapiens
MALRVLLLTALTLCHGFNLDTENAMTFQENARGFGQSVVQLQGSRVVVGAPQEIVAANQRGSLYQCDYSTGSCEPIRLQVPVEAVNMSLGLSLAATTSPPQLLACGPTVHQTCSENTYVKGLCFLFGSNLRQQPQKFPEALRGCPQEDSDIAFLIDGSGSIIPHDFRRMKEFVSTVMEQLKKSKTLFSLMQYSEEFRIHFTFKEFQNNPNPRSLVKPITQLLGRTHTATGIRKVVRELFNITNGARKNAFKILVVITDGEKFGDPLGYEDVIPEADREGVIRYVIGVGDAFRSEKSRQELNTIASKPPRDHVFQVNNFEALKTIQNQLREKIFAIEGTQTGSSSSFEHEMSQEGFSAAITSNGPLLSTVGSYDWAGGVFLYTSKEKSTFINMTRVDSDMNDAYLGYAAAIILRNRVQSLVLGAPRYQHIGLVAMFRQNTGMWESNANVKGTQIGAYFGASLCSVDVDSNGSTDLVLIGAPHYYEQTRGGQVSVCPLPRGQRARWQCDAVLYGEQGQPWGRFGAALTVLGDVNGDKLTDVAIGAPGEEDNRGAVYLFHGTSGSGISPSHSQRIAGSKLSPRLQYFGQSLSGGQDLTMDGLVDLTVGAQGHVLLLRSQPVLRVKAIMEFNPREVARNVFECNDQVVKGKEAGEVRVCLHVQKSTRDRLREGQIQSVVTYDLALDSGRPHSRAVFNETKNSTRRQTQVLGLTQTCETLKLQLPNCIEDPVSPIVLRLNFSLVGTPLSAFGNLRPVLAEDAQRLFTALFPFEKNCGNDNICQDDLSITFSFMSLDCLVVGGPREFNVTVTVRNDGEDSYRTQVTFFFPLDLSYRKVSTLQNQRSQRSWRLACESASSTEVSGALKSTSCSINHPIFPENSEVTFNITFDVDSKASLGNKLLLKANVTSENNMPRTNKTEFQLELPVKYAVYMVVTSHGVSTKYLNFTASENTSRVMQHQYQVSNLGQRSLPISLVFLVPVRLNQTVIWDRPQVTFSENLSSTCHTKERLPSHSDFLAELRKAPVVNCSIAVCQRIQCDIPFFGIQEEFNATLKGNLSFDWYIKTSHNHLLIVSTAEILFNDSVFTLLPGQGAFVRSQTETKVEPFEVPNPLPLIVGSSVGGLLLLALITAALYKLGFFKRQYKDMMSEGGPPGAEPQ

(19) United States
(12) Patent Application Publication
(10) Pub. No.: US 2013/0079309 A1
(43) Pub. Date: Mar. 28, 2013

(54) Method Of Treating Acute Lung Injury Using Sphingosine 1 Phosphate Analogs Or Sphingosine 1 Phosphate Receptor Agonists

(71) Applicants: Joe G.N. Garcia, Chicago, IL (US); Steven M. Dudek, Chicago, IL (US)

(72) Inventors: Joe G.N. Garcia, Chicago, IL (US); Steven M. Dudek, Chicago, IL (US)

(73) Assignee: The Board of Trustees of the University of Illinois, Urbana, IL (US)

(21) Appl. No.: 13/582,627

(22) PCT Filed: Mar. 3, 2011
(86) PCT No.: PCT/US11/27074

§ 371 Date: Dec. 13, 2012

Related U.S. Application Data
(60) Provisional application No. 61/309,948, filed on Mar. 3, 2010.

(51) Int. Cl. A61K 31/662; A61K 31/4245; A61K 31/137

(52) U.S. Cl. CPC A61K 31/662; A61K 31/137; A61K 31/4245
USPC 514/114; 514/653; 514/364; 73/61.43

(57) ABSTRACT

The invention provides methods for treating or reducing the risk of developing acute lung injury manifested by increased vascular permeability. Also provided are pharmaceutical compositions comprising an FTY720 analog or derivative and/or SEW 2871 for use in the disclosed methods. The invention also provides methods for treating or reducing the risk of developing acute lung injury resulting from dysregulation of ceramide/sphingolipid pathway, more specifically, acute lung injury resulting from radiation.

This application relates to, and claims the benefit of priority to U.S. Provisional Application 61/309,948, filed Mar. 3, 2010, the disclosure of which is hereby incorporated by reference in its entirety.

This invention was made with government support under grant No. HL058064 awarded by the National Institute of Health. The government has certain rights in the invention.

### BACKGROUND OF THE INVENTION

Vascular leakage associated with disruption of the endothelial cell (EC) barrier, and subsequent extravasation into the airspaces of the lung are hallmarks of acute lung injury (ALI). Disruption of pulmonary vascular EC monolayer in the lung results in flooding of interstitial and alveolar compartments with fluid, protein, and inflammatory cells, resulting in respiratory failure. See Dudek and Garcia, 2001, J. Appl. Physiol. 91:1487-1500. It has been shown that sphingosine 1 phosphate (S1P), a platelet-derived sphingolipid, can induce EC cytoskeletal rearrangements via Gᵢ-coupled S1P receptor (S1P₁R), which leads to augmented EC monolayer integrity. See Garcia, 2001, J. Clin. Invest. 108:689-701. Infection and sepsis are often the triggering event of ALI, and SIP has been shown to attenuate bacterial endotoxin lipopolysaccharide (LPS)-induced sepsis in murine and canine ALI models. See Wheeler and Bernard, 2007, Lancet 369:1553-64; McVerry et al., 2004, Am. J. Respir. Crit. Care Med. 170:987-993; and Peng et al., 2004, Am. J. Respir. Crit. Care Med. 169:1245-51. S1P is formed by phosphorylation of sphingosine via sphingosine kinase (SphK); acylation of sphingosine produces ceramide, a pro-apoptotic molecule in the lung. See Petrache et al., 2005, Nat. Med. 11:491-8. In addition, ceramide can also be produced from sphingomyelin via enzymatic activities of sphingomyelinases. See Marchesini et al., 2004, Biochem Cell Biol. 82:27-44. Increased lung vascular permeability has been linked to acid sphingomyelinase-dependent production of ceramide in murine ALI. See Goggel et al., 2004, Nat. Med. 10:155-60. On the other hand, dihydrosphingosine (a precursor of ceramide) is converted by sphingosine kinase 1 to dihydrosphingosine 1-phosphate (DHS1P), is a pro-survival molecule like SIP. See Berdyshev et al., 2006, Cell Signal. 18:1779-92.

Use of S1P as an ALI therapy is not straightforward, however, being hampered by its myriad of effects in vivo. For example, S1P binding to S1P₃ receptor (S1P₃R) in the heart can lead to cardiac toxicity, primarily bradycardia. See Forrest et al., 2004, J. Pharmacol. Exp. Ther. 309:758-68; Hale et al., 2004, Bioorg. Med. Chem. Lett. 15:4470-74. In addition, S1P can stimulate smooth muscle contraction in the human airway and exacerbate airway obstruction in asthmatics. See Rosenfeldt et al., 2003, FASEB J. 17:1789-99; Roviezzo et al., 2007, Am. J. Respir. Cell Mol. Biol. 36:757-62. Further, despite S1P's barrier-enhancing potential, intratracheal administration of S1P can produce edema through disruption of the epithelial barrier via ligation of SIP with S1P₃R and subsequent Rho activation. See Gon et al., 2005, Proc. Natl. Acad. Sci. USA 102:9270-75. Even in the vasculature, high doses (>10 μM) of S1P can disrupt EC monolayer integrity. Camp et al., 2009, J. Pharmcol. Exp. Ther. 331:54-64. Thus, S1P has a rather limited therapeutic window for its barrier-enhancing properties.

FTY720 (fingolimod, marketed by Novartis as GILENYA™) is a compound derived from myriocin, a fungal sphingosine-like metabolite that is currently in clinical trials for use as an immunosuppressant. FTY720 (2-amino-2-[2-(4-octylphenyl)ethyl]propane-1,3-diol) has been shown to be a S1PR agonist, and considerable efforts have been devoted to investigate the therapeutic potential of FTY720 in inflammatory lung injury. Initial results have demonstrated that FTY720 exhibits potent barrier-enhancing properties both in vitro and in vivo. See Sanchez et al., 2003, J. Biol. Chem. 278: 47281-47290; Peng et al., 2004, Am J Respir Crit Care Med 169: 1245-1251: Dudek et al., 2007, Cell Signal 19: 1754-1764. For example, a single intraperitoneal injection of FTY720 has been shown to attenuate murine LPS induced-pulmonary injury measured 24 hours after LPS administration. See Peng et al., 2004, Id.

Like S1P, FTY720 has certain properties that limit its therapeutic utility in patients with ALI. The effectiveness of FTY720 as an immunosuppressant relates to its ability to induce lymphopenia via down-regulation of lymphocyte S1P₁R signaling, which may be detrimental, inter alia, in patients with ALI triggered by infection or sepsis. See Kovarik et al., 2004, Ther Drug Monit 26: 585-587; Matloubian et al., 2004, Nature 427: 355-360.

Acute lung injury can have many causes, including for example endotoxin-induced acute lung injury associated with infection, trauma in the lung, and radiation-induced lung injury (RILI).

Endotoxin exposure often accompanies Gram-negative bacterial infection and can cause acute lung injury. For example, lipopolysaccharide (LPS) associated with the E. coli outer membrane elicits a variety of inflammatory responses in mammals. In particular, endotoxin-associated effects in the lungs include diffuse lung inflammation and injury of the pulmonary vascular endothelium (Brigham and Meyrick, 1986, Am Rev Respir Dis. 133:913-27). Endotoxin has been shown to directly damage endothelial cells in vitro and in vivo. Endotoxin-associated lung injury has been linked to the presence of granulocytes, lymphocytes, and macrophages in lung tissue, which may participate in the response either directly or by directing cell traffic. In addition, endotoxin-associated ALI is likely mediated at least in part by generation of free radicals. Inflammatory cells, especially neutrophils, are one source of reactive oxygen species, but endotoxin may also stimulate generation of free radicals within lung cells. Multiple mediators of endotoxin-associated ALI have been proposed (see Brigham and Meyrick, 1986, Id.); an effective treatment, however, is still lacking.

Radiation elicits lung damage. Radiation-induced lung injury (RILI) is a general term referring to damage associated with exposure to ionizing radiation to the lungs, which is the most radiosensitive organ. RILI most commonly occurs in patients receiving radiation therapy to treat thoracic cancer. RILI is a disabling and potentially fatal, dose-limiting toxicity of thoracic radiotherapy for lung cancer, breast cancer, lymphoma, thymoma, esophageal cancer and total body irradiation. See Vujaskovic et al., 2000, Semin Radiat Oncol 10:296-307; Carruthers et al., 2004, British Journal of Cancer 90:2080-2084. Although RILI can be self-limited, it may progress to overt lung injury and lung failure associated with significant morbidity or death. See Rodrigues et al., 2004, Radiother Oncol 71:127-138.

The molecular basis of RILI remains both controversial and unclear. See Roberts et al., 1993, Ann Intern Med 118:696-700. RILI is associated with increased generation of reactive oxygen and nitrogen species, secretion of inflammatory cytokines and chemokines, and inflammatory cell recruitment into the lung parenchyma. RILI is symptomatically characterized by a lung inflammatory response consistent with other forms of ALI in which EC barrier dysfunction is a cardinal feature.

To date, therapeutic strategies for RILI have largely been designed to ameliorate the acute effects of radiation by neutralizing pro-inflammatory cytokines or attenuating inflammatory cell infiltration. See Travis, 1980, Int J Radiat Oncol Biol Phys 6:1267-1269. The pluripotential nature of these cytokines and multifaceted signaling pathways, however, complicate their utility as viable targets. Moreover, corticosteroid therapy, commonly utilized for RILI, suffers from limited efficacy, serious side effects, and the potential for fatal “recall” pneumonitis when abruptly discontinued. See Kwok and Chan, 1998, Can Respir J 5:211-214. Alternative treatment strategies, such as anticoagulation or angiotensin converting enzyme inhibitors, have failed to provide compelling clinical benefit. See Molteni et al., 2000, Int J Radiat Biol 76:523-532. Thus, a need exists for effective long-term therapy, especially therapy with minimal or reduced side effects, to improve survival and disease management of patients with these acute lung injuries.

### SUMMARY OF THE INVENTION

Provided herein are methods for treating or reducing the risk of developing acute lung injury, particularly acute lung injury associated with radiation, infection, trauma, and other environmental or medical treatment-associated insults to the lung. In one aspect, the invention provides methods for treating or reducing the risk of developing radiation-induced acute lung injury in a mammal comprising the step of administering to a mammal in need thereof an effective amount of an FTY720 derivative or analog or SEW 2871. In certain embodiments, the mammal is subjected to thoracic radiation therapy. In certain other embodiments, the FTY720 derivative or analog or SEW 2871 is administered before, after or concurrently with radiation. In certain particular embodiments, the FTY720 derivative or analog is the (S)-enantiomer of FTY720 phosphonate.

In a further aspect, the invention provides methods of reducing weight loss or hair loss associated with radiation therapy in a mammal comprising the step of administering to a mammal in need thereof an FTY720 derivative or analog in an amount sufficient to reduce weight loss or hair loss associated with radiation therapy. In certain particular embodiments, the radiation therapy is thoracic radiation therapy. In some embodiments, the FTY720 derivative or analog is administered before, concurrently with or after the mammal is undergoing radiation therapy. In certain particular embodiments, the FTY720 derivative or analog is the (S)-enantiomer of FTY720 phosphonate.

In another aspect, the invention provides methods of treating or reducing the risk of developing acute lung injury in a mammal comprising the step of administering to a mammal in need thereof an effective amount of an FTY720 derivative or analog or SEW 2871. In certain particular embodiments, the acute lung injury is induced by endotoxin. In particular embodiments, the endotoxin is lipopolysaccharide (LPS). In certain other embodiments, the FTY720 derivative or analog or SEW2871 is administered to the mammal before, after or concurrently with the exposure of the mammal to endotoxin. In certain particular embodiments, the FTY720 derivative or analog is the (S)-enantiomer of FTY720 phosphonate.

In certain embodiments of the above aspects, the administration of an FTY720 derivative or analog or SEW 2871 reduces vascular leakage or vascular permeability in the mammal, wherein vascular leakage or vascular permeability occurs as a result of acute lung injury. In certain other embodiments, the administration of an FTY720 derivative or analog or SEW 2871 reduces BAL protein levels in the mammal, wherein the BAL protein levels increase as a result of acute lung injury. In certain particular embodiments, the administration of an FTY720 derivative or analog or SEW 2871 reduces BAL cell count in the mammal, wherein BAL cell count increases as a result of acute lung injury. In other particular embodiments, the administration of an FTY720 derivative or analog or SEW 2871 increases alveolar cell integrity or increases endothelial cell integrity in the mammal, wherein alveolar cell integrity or endothelial cell integrity decreases as a result of acute lung injury. In other embodiments, the administration of an FTY720 derivative or analog or SEW 2871 reduces lung inflammation in the mammal, wherein lung inflammation occurs as a result of acute lung injury. In certain embodiments, the administration of an FTY720 derivative or analog or SEW 2871 reduces dysregulation of the ceramide/sphingolipid metabolic pathway in the lung of the mammal, wherein the dysregulation of the ceramide/sphingolipid metabolic pathway in the lung occurs as a result of acute lung injury. In certain other embodiments, the dysregulation of the ceramide/sphingolipid metabolic pathway in the lung is indicated by decreased combined levels of sphingosine 1 phosphate (S1P) and dihydro-S1P (DHS1P) in a sample from the lung. In certain particular embodiments, the dysregulation of the ceramide/sphingolipid metabolic pathway in the lung is indicated by increased levels of ceramide in a sample from the lung. In certain particular embodiments, the sample from the lung is a lung tissue sample, a BAL fluid sample, or a plasma sample, preferably a BAL fluid sample. The invention also provides methods resulting in a plurality or substantially all of these effects.

In another aspect, the invention provides methods of treating or reducing the risk of developing acute lung injury in a mammal resulting from dysregulation of the ceramide/sphingolipid metabolic pathway, comprising the step of administering to a mammal in need thereof an FTY720 derivative or analog or SEW 2871 in an amount capable of reversing dysregulation of the ceramide/sphingolipid metabolic pathway. In certain embodiments, the dysregulation of the ceramide/sphingolipid pathway occurs in the lung. In certain embodiments, the acute lung injury is induced by radiation, and in other embodiments, the acute lung injury is induced by endotoxin. In particular embodiments, the endotoxin is lipopolysaccharide (LPS). In certain embodiments, the FTY720 derivative or analog or SEW 2871 is administered to the mammal before the mammal is exposed to radiation or endotoxin. In other embodiments, the FTY720 derivative or analog or SEW2871 is administered to the mammal after the mammal is exposed to radiation or endotoxin. In particular embodiments, the FTY720 derivative or analog or SEW2871 is administered to the mammal concurrently with the exposure of the mammal to radiation. In certain particular embodiments, the FTY720 derivative or analog is the (S)-enantiomer of FTY720 phosphonate.

In a further aspect, the invention provides methods of reducing vascular leakage or vascular permeability in the lung, or reducing the risk of developing vascular leakage or increased vascular permeability in the lung of a mammal comprising the step of administering to a mammal in need thereof an effective amount of an FTY720 derivative or analog or SEW 2871. In some embodiments, the vascular leakage or vascular permeability in the lung is due to radiation or endotoxin. In certain embodiments, the endotoxin is lipopolysaccharide (LPS). In some embodiments, the FTY720 derivative or analog is administered to the mammal before the mammal is exposed to radiation or endotoxin. In other embodiments, the FTY720 derivative or analog or SEW2871 is administered to the mammal after the mammal is exposed to radiation or endotoxin. In particular embodiments, the FTY720 derivative or analog or SEW2871 is administered to the mammal concurrently with the exposure of the mammal to radiation. In certain particular embodiments, the FTY720 derivative or analog is the (S)-enantiomer of FTY720 phosphonate.

In yet another aspect, the invention provides methods of reducing acute lung inflammation in a mammal comprising the step of administering to a mammal in need thereof an effective amount of an FTY720 derivative or analog or SEW 2871. In some embodiments, the acute lung inflammation is due to radiation or endotoxin, and in particular embodiments, the endotoxin is lipopolysaccharide (LPS). In some embodiments, the FTY720 derivative or analog or SEW 2871 is administered to the mammal before the mammal is exposed to radiation or endotoxin. In other embodiments, the FTY720 derivative or analog or SEW2871 is administered to the mammal after the mammal is exposed to radiation or endotoxin. In particular embodiments, the FTY720 derivative or analog or SEW2871 is administered to the mammal concurrently with the exposure of the mammal to radiation. In certain particular embodiments, the FTY720 derivative or analog is the (S)-enantiomer of FTY720 phosphonate.

In an additional aspect, the invention provides methods of increasing alveolar cell integrity or increasing endothelial cell integrity in a mammal comprising the step of administering to a mammal in need thereof an effective amount of an FTY720 derivative or analog or SEW 2871. In some embodiments, the alveolar cell or endothelial cell integrity is reduced due to radiation or endotoxin; and in particular embodiments, the endotoxin is lipopolysaccharide (LPS). In some embodiments, the FTY720 derivative or analog or SEW 2871 is administered to the mammal before the mammal is exposed to radiation or endotoxin. In other embodiments, the FTY720 derivative or analog or SEW2871 is administered to the mammal after the mammal is exposed to radiation or endotoxin. In particular embodiments, the FTY720 derivative or analog or SEW2871 is administered to the mammal concurrently with the exposure of the mammal to radiation. In certain particular embodiments, the FTY720 derivative or analog is the (S)-enantiomer of FTY720 phosphonate.

In another aspect, the invention provides methods of reducing BAL protein levels or BAL cell count in a mammal comprising the step of administering to a mammal in need thereof an effective amount of an FTY720 derivative or analog or SEW 2871. In some embodiments, the BAL protein levels or BAL cell count in the mammal is increased due to radiation or endotoxin; in particular embodiments, the endotoxin is lipopolysaccharide (LPS). In some embodiments, the FTY720 derivative or analog or SEW2871 is administered to the mammal before the mammal is exposed to radiation or endotoxin. In other embodiments, the FTY720 derivative or analog or SEW2871 is administered to the mammal after the mammal is exposed to radiation or endotoxin. In particular embodiments, the FTY720 derivative or analog or SEW2871 is administered to the mammal concurrently with the exposure of the mammal to radiation. In certain particular embodiments, the FTY720 derivative or analog is the (S)-enantiomer of FTY720 phosphonate.

In yet another aspect, the invention provides methods of treating or reducing the risk of developing radiation-induced lung injury (RILI) in a mammal comprising the step of administering to a mammal in need thereof an effective amount of an FTY720 derivative or analog or SEW 2871. In some embodiments, the FTY720 derivative or analog or SEW 2871 is administered before radiation. In other embodiments, the FTY720 derivative or analog or SEW 2871 is administered after radiation. In particular embodiments, the FTY720 derivative or analog or SEW2871 is administered to the mammal concurrently with the exposure of the mammal to radiation. In certain particular embodiments, the FTY720 derivative or analog is the (S)-enantiomer of FTY720 phosphonate.

In further aspects, the invention provides uses of an FTY720 analog or derivative or SEW2871 for the preparation of a medicament for the treatment or prevention of acute lung injury, particularly radiation-induced lung injury, endotoxin-induced lung injury, acute lung inflammation, or acute lung injury resulting from dysregulation of the ceramide/sphingolipid metabolic pathway. In additional aspects, the invention provides uses of an FTY720 analog or derivative or SEW2871 for reducing acute lung inflammation, increasing alveolar cell integrity or increasing endothelial cell integrity, reducing BAL protein levels or BAL cell count, or reducing weight loss or hair loss associated with radiation therapy. In certain particular embodiments, the FTY720 derivative or analog is the (S)-enantiomer of FTY720 phosphonate.

In particular embodiments of any and all of the aspects of the invention, the FTY720 analog or derivative is the (R)— or (S)-enantiomer of FTY720 phosphonate, the (R)- or (S)-enantiomer of FTY720-enephosphonate, or the (R)- or (S)-enantiomer of FTY720 regioisomer. In certain embodiments, the FTY720 analog or derivative is the (S)-enantiomer of FTY720-phosphonate. In other embodiments of any of the aspects of the invention, the mammal is a human.

In certain embodiments, the invention provides pharmaceutical dosage forms comprising an FTY720 analog or derivative or SEW2871 in an amount of about 0.7 mg/dosage unit-about 500 mg/dosage unit and a pharmaceutically acceptable carrier. In some embodiments, the FTY720 analog or derivative or SEW2871 is present in an amount from about 0.7 mg/dosage unit-about 70 mg/dosage unit. In other embodiments, the FTY720 analog or derivative or SEW2871 is present in an amount from about 70 mg/dosage unit-about 500 mg/dosage unit. In certain embodiments, the FTY720 analog or derivative is the (R)- or (S)-enantiomer of FTY720 phosphonate, the (R)- or (S)-enantiomer of FTY720-enephosphonate, or the (R)- or (S)-enantiomer of FTY720 regioisomer. In certain particular embodiments, the FTY720 analog or derivative is the (R)- or (S)-enantiomer of FTY720 phosphonate; in other particular embodiments, the FTY720 analog or derivative is the (S)-enantiomer of FTY720 phosphonate.

In another aspect, the invention provides methods of diagnosing radiation-induced lung injury in a mammal comprising the step of assaying a sample from a mammal after exposure to radiation to detect levels of sphingosine-1-phosphate (S1P), dihydro-S1P (DHS1P), or ceramide wherein lung injury is diagnosed when the combined levels of S1P and DHS1P are reduced in the sample from the mammal as compared to the S1P levels or DHS1P levels in a sample from a control mammal or when the ceramide levels are increased in the sample from the mammal as compared to the ceramide levels in the sample from the control mammal In certain particular embodiments, lung injury is diagnosed when the ceramide levels are increased in a sample from the mammal as compared to the ceramide levels in a sample from the control mammal or from the mammal at a different time, for example prior to radiation exposure. As practiced according to the methods of the invention, samples from a mammal are taken after exposure to radiation, particularly and advantageously four-six weeks after the mammal is exposed to radiation. In some embodiments, the sample is a lung tissue sample, a BAL fluid sample, or a plasma sample.

It was unexpectedly discovered by the inventors of the instant application that although FTY720 has been shown to reduce LPS-induced BAL protein levels and BAL cell count, FTY720 is ineffective in treating radiation-induced ALI. The present invention provides methods of treating or reducing the risk of developing acute lung injury, especially radiation-induced lung injury, comprising the step of administering to a mammal in need thereof an effective amount of an FTY720 analog or derivative or SEW 2871. In addition, the inventors unexpectedly discovered the benefit of using an FTY720 analog or derivative in protecting a mammal from the side effects, such as weight loss or hair loss, associated with thoracic radiation therapy. Advantageously, the present invention provides methods for reducing weight loss and hair loss associated with thoracic radiation therapy in a mammal, comprising administering to a mammal in need thereof an FTY720 analog or derivative.

Specific embodiments of the present invention will become evident from the following more detailed description of certain preferred embodiments and the claims.

### BRIEF DESCRIPTION OF THE DRAWINGS

FIG. 1 shows BAL cell counts (FIG. 1A; n=10 animals/experimental group, # p<0.01 compared to controls), BAL cell type (FIG. 1B; # p<0.01 compared to controls, PMNs, polymorphonuclear (PMN) leukocytes or neutrophils) and cytokine expression (FIG. 1C; n=4 animals/experimental group, # p<0.01) in a preclinical murine model of radiation-induced lung injury (RILI); # p<0.01 compared to controls.

FIG. 2A shows BAL protein concentrations in RILI mice; n=7 animals/experimental group, * p<0.05 and #p<0.01 compared to respective controls. FIG. 2B shows extravasation of intravenously delivered Evans Blue Dye (EBD) in RILI mice; n=4, * p<0.05, # p<0.01. FIG. 2C shows photographs of EBD extravasation as a measure of lung vascular leak after exposure to 25 Gy thoracic radiation.

FIG. 3A shows hematoxylin and eosin staining of murine lung sections 4, 6, 8, and 12 weeks post-irradiation. FIG. 3B shows immunohistochemical localization of nitrotyrosine in the lungs of RILI mice 4 weeks post-irradiation. Compared to controls, strong immunoreactivity of nitrotyrosine at 4 wks post-irradiation was observed in the alveolar epithelium (left arrow), pneumocytes and airway epithelium (right arrow).

FIG. 4A shows the experimental design for simvastatin therapy of RILI. Mice received simvastatin (10 mg/kg body weight, 3×/wk) or vehicle beginning one week prior to radiation (25 Gy, single dose) and continuing up to 6 wks post-irradiation with collection of BAL samples (protein, cell counts, biomarkers), and tissue (Evans blue dye, microarray, histology) at the interval indicated. FIG. 4B shows percent body weight change in mice receiving simvastatin or no treatment, with or without irradiation; n=5 animals/experimental group; ## p<0.01 compared to RILI alone; * p<0.05 and # p<0.01 compared to controls. BAL cell counts (FIG. 4C), BAL protein (FIG. 4D), and EBD extravasation (FIG. 4E) were determined from murine lungs after simvastatin treatment at 6 wks; n=5 animals/experimental group, * p<0.05 and # p<0.01 compared to controls; ** p<0.05 compared to RILI alone. FIG. 4F shows the levels of BAL pro-inflammatory cytokines at 4 wks post-irradiation; n=5 animals/experimental group, #, p<0.01 compared to controls and ##, p<0.01 compared to radiation alone.

FIG. 5 shows the effects of simvastatin on murine RILI lungs via histology (FIG. 5A) and ViSen FMT imaging (FIG. 5B).

FIG. 6A shows dynamic changes in radiation-induced lung gene dysregulation; expression levels across multiple time-points post-RILI were clustered and displayed by dChip software. Red, white and blue color indicates expression level above, at or below the average level of corresponding gene, respectively. The genes in the right-hand side of the drawing in Cluster 1 show down-regulated genes at 6 weeks, and the genes in Cluster 2 show up-regulated genes at 6 weeks. FIG. 6B shows hierarchical clustering of differentially expressed genes (control vs. RILI) across all 6 week samples identified by Significance Analysis of Microarrays. Genes in Cluster 1 show down-regulation in RILI at 6 weeks compared with control, and genes in Cluster 2 show up-regulation in RILI at 6 weeks compared with control. Genes in Cluster 1 in RILI+Simva and Simva alone generally show similar patterns as the control. Genes in Cluster 2 in RILI+Simva and Simva alone show patterns closer to control with some up-regulation, though to a less extent as compared with RILI only. Genes were displayed by dChip software, classified into two clusters (down-regulated genes-Cluster 1, up-regulated genes-Cluster 2). Blue, white and red colors represent expression levels below, at and above the average level of the corresponding gene, respectively.

FIG. 7A shows radiation-induced deregulated proteins identified by “Single Network Analysis of Proteins” (SNAP). False discovery rates of 0.0001% (large circles), 5% (medium size circles), and 10% (small circles). FIG. 7B shows focal adhesion pathway genes that were significantly deregulated by RILI and attenuated by simvastatin (Benjamini-Hochberg corrected hypergeometric p-value=0.0035). Control: left bar; Radiation, middle bar; Simva-Radiation, right bar. FIG. 7C shows canonical pathways deregulated by irradiation in vehicle- and simvastatin-treated animals (left and right bars, respectively). Significance was determined by the single-sided Fisher exact test at p-value<0.05, as indicated by the red threshold line in the graph. FIG. 7D shows real-time qPCR validation of fold change in expression of deregulated Ccna2 and Cdc2 genes (RNA isolated from lung homogenates) following exposure to radiation, simvastatin or both exposures. The significance of gene dysregulation was determined by two-group comparison using a t-test; ##, p=0.01 compared to radiated controls.

FIG. 8A shows Western blotting of lung homogenates from RILI-challenged mice (25 Gy), demonstrating increases in SphK1 and Sphk2 but not S1P lyase (S1PL) expression at 6 weeks. FIG. 8B shows densitometric analysis of the Western blots; n=3/group; * p<0.05 compared to control, ** p<0.05 compared to RILI alone.

FIG. 9 shows S1P (FIGS. 9A-C) and ceramide (FIGS. 9D-F) levels analyzed from lung homogenates, BAL fluids and plasma collected from RILI (25Gy) challenged mice (n=3/group), at different time points (1 hr to 12 wks post radiation) using LC-MS/MS. FIGS. 9G-I show the ratio of ceramide to cumulative S1P and DHS1P levels in lung homogenates, BAL fluid, and plasma; * p<0.05 RILI vs. control. FIGS. 9J-L show the ratio of ceramide to cumulative S1P and DHS1P levels in lung homogenates, BAL fluid, and plasma in mice that received simvastatin (10 mg/kg body weight, 3×/wk) or vehicle beginning 1 week prior to radiation (25 Gy, single thoracic dose) and up to 6 weeks post-irradiation; * p<0.05 RILI vs. control; ** p<0.01 RILI+Simva vs. RILI.

FIG. 10 shows BAL fluid protein content and cell counts in RILI-challenged (25 Gy) SphK^{-/-} mice at 6 weeks (FIGS. 10A-B), RILI-challenged (10 Gy) S1PR1^{+/-} mice at 4 weeks, (FIGS. 10C-D), RILI-challenged (20 Gy) S1PR2^{-/-} mice after 6 weeks (FIGS. 10E-F), and RILI-challenged (20 Gy) S1PR3^{-/-} mice after 6 weeks (FIGS. 10G-H), compared to respective wild type RILI-challenged control animals; n=3-5/group, * p<0.05 compared to wild type controls, ** p<0.05 compared to RILL challenged wild type mice.

FIG. 11 shows protein levels and cell counts from BAL fluid collected from C57B1/6 mice pretreated with 0.01 or 0.1 mg/kg (i.p.) (S)-FTY720-phosphonate (fTyS) (FIGS. 11A-B), SEW2871 (FIGS. 11C-D), or FTY720 (FIGS. 11E-F) 2×/wk beginning one week before irradiation (20 Gy); n=5/group, * p<0.05 compared to uninjured controls, ** p<0.05 compared to RILI controls.

FIG. 12A shows hematoxylin and eosin staining of lung sections from mice 6 weeks after administration of a single dose of thoracic radiation (25 Gy), and from similarly RILI-challenged mice (25 Gy) treated with FTY720, SEW, or (S)-FTY720-phosphonate (0.1 mg/kg, i.p., administered 2×/wk beginning one week prior to irradiation); arrows: prominent influxes of inflammatory cells. FIG. 12B shows the results of separate experiments, in which RILI-challenged mice (25 Gy) were injected with an intravascular probe (Integrisense⁶⁸⁰) 6 weeks post radiation and administered with no treatment, FTY720, SEW, or (S)-FTY720-phosphonate (0.1 mg/kg), then subjected to ViSen FMT imaging 6 hrs later. FIG. 12C shows the quantification of the scanning results.

FIG. 13 shows results of hierarchical clustering of genes dysregulated by radiation at 6 weeks across experimental conditions as identified by Significance Analysis of Microarrays. Genes were displayed by dChip software and classified into two clusters (down-regulated genes and up-regulated genes). Blue, white and red colors represent expression levels below, at and above the average level of the corresponding gene, respectively. The darker areas in the upper portion of radiation+vehicle-treated lanes represents upregulated genes, the expression levels of which were substantially reverted near to the control levels in the radiation+fTyS and radiation+SEW groups. The darker areas in middle portion of the radiation+vehicle group represent downregulated genes, the expression levels of which were reverted close to the control levels in the radiation+fTyS and the radiation+SEW groups. In the radiation+FTY group, however, the darker cluster of genes in the upper portion remains largely upregulated, and the darker cluster of genes in the middle portion remains largely downregulated, similar to the radiation+vehicle group. The Figure also shows many genes whose expression does not change (represented in white) or that changes weakly (the areas with lighter intensity).

FIG. 14 shows results of principal component analysis (PCA) of genes dysregulated in murine RILI and effects of S1P analogs. FIG. 14A shows a PCA 3D scatter plot in which each triangle represents a sample, control, radiation alone, radiation+(S)-FTY720-phosphonate, radiation+SEW, and radiation+FTY720 are indicated. FIG. 14B shows corresponding principal component changes, expressed as the linear gene-specific weight over the expression of all analyzed genes; n=3/group, *p<0.05.

FIG. 15 shows percent change in weight (FIG. 15A), BAL cell counts (FIG. 15B), and BAL protein levels (FIG. 15C) in RILI mice in response to treatment with irradiation alone, irradiation and simvastatin, irradiation and (S)-FTY720-phosphonate (fTys), and irradiation and FTY720 (FTY). In FIG. 15A, n=5 and p<0.01; in FIGS. 15B-C, n=5/group.

FIG. 16 show direct comparisons of BAL cell counts (FIG. 16A) and BAL protein levels (FIG. 16B) for RILI mice treated with simvastatin and (S)-FTY720-phosphonate (Tysip). For these experiments, n=5/group. BAL cell count and BAL protein level studies were done as separate, independent experiments.

FIGS. 17A-B show protein expression levels of S1P receptor 1 (S1PR1) in response to addition of S1P, FTY720 (FTY), (S)-FTY720-phosphonate (15), (R)-FTY720-phosphonate (1R), SEW2871 (SEW), and phosphorylated FTY720 (p-FTY).

FIGS. 18A-B show protein expression levels of S1P receptor 1 (S1PR1) in response to addition of S1P, FTY720 (FTY), (S)-FTY720-phosphonate (1S), (R)-FTY720-phosphonate (1R), SEW, and phosphorylated FTY720 (p-FTY), combined with addition of the proteasome inhibitor MG132.

FIGS. 19A-B show ubiquitination of S1P receptor 1 (S1PR1) by S1P, FTY720 (FTY), (S)-FTY720-phosphonate (1S), (R)-FTY720-phosphonate (1R), SEW, and phosphorylated FTY720 (p-FTY) after 1 hour or 2 hours, respectively.

FIG. 20 shows the results of a Tango™ EDG-1 cell-based assay to detect activation of beta-arrestin by S1P, FTY720 (FTY), (S)-FTY720-phosphonate (fTyS), SEW, and phosphorylated FTY720 (p-FTY). Mean of n=3±S.E. * p<0.05 fTyS vs. S1P, FTY720, SEQ and p-FTY.

FIG. 21 shows Kaplan-Meier curves of survival in mice receiving bleomycin alone (dotted line), bleomycin+FTY720 (gray line), or bleomycin+(S)-FTY720-phosphonate (fTyS) (black line). N=6 animals per group.

FIG. 22 shows BAL fluid protein levels in mice treated with FTY720 or (S)-FTY720-phosphonate (fTyS) 14 days after the animals were administered with bleomycin. For comparison, BAL protein levels in control (no bleomycin treatment) mice were ˜200. Only 1 out of the 6 bleomycin+FTY720 mice survived, while 5 out of 6 bleomycin+fTyS mice survived, to 14 days to obtain this measurement.

FIG. 23 shows S1P receptor 1 (S1PR1) levels in the lungs of mice treated with FTY720 or (S)-FTY720-phosphonate (fTyS). Lung homogenates were collected 14 days after bleomycin instillation. S1PR1 protein expression was determined by Western blotting (representative blots shown), quantified by densitometry, and normalized to actin concentration in the samples. Mean±S.D. is shown. N=2-3 animals per group.

FIG. 24 shows the chemical structures for S1P, FTY720 (FTY), FTY720-phosphate (p-FTY), (S)-FTY720-phosphonate (1S or tysiponate or fTyS), (R)-FTY720-phosphonate (compound 1R), (S)-FTY720-enephosphonate (compound 2S), (R)-FTY720-enephosphonate (compound 2R), (S)-FTY720 regioisomer (compound 3S), (R)-FTY720 regioisomer (compound 3R), and SEW2871 (SEW).

FIG. 25 shows differential effects of FTY720 analogs on endothelial cell barrier function in vitro. FIG. 25A shows a transendothelial electrical resistance (TER) tracing, generated from HPAEC plated on gold electrodes stimulated with 1 μM S1P (black line), FTY720 (red), 1R (blue), 2R (green), or 3R (purple) at time=0; the TER tracing represents pooled data (±S.E.M.) from four independent experiments. Bar graphs depict pooled TER data from HPAEC stimulated at 1 (FIG. 25B) or 10 μM (FIG. 25C) with S1P, FTY720, 1R, 1S, 2R, 2S, 3R, or 3S as indicated. The data are expressed as maximal percentage TER change (±S.E.M.) obtained within 60 min. Positive values indicate barrier enhancement. Negative values indicate barrier disruption. n=3-5 independent experiments per condition; *, p<0.01 versus other conditions.

FIG. 26 shows the effects of FTY720 analogs on Transwell endothelial cell permeability; p<0.01 versus unstimulated condition.

FIG. 27 shows cytoskeletal rearrangement induced by FTY720 analogs by immunofluorescence (FIG. 27A; arrows indicate increased cortical actin) and Western blot (FIG. 27B). Note that all wells represent equal loading of total proteins. Experiments were independently performed in triplicate with representative blots shown.

FIG. 28 shows the effects of FTY720 analogs on intracellular calcium release. Cultured HPAEC were stimulated with methanol vehicle or 1 μM S1P, FTY720, 1R, 2R, or 3R at time 0, and intracellular calcium levels were measured as fold change in [Ca²⁺] relative to 60-s average before treatment, as determined by Fura-2. n=3 independent experiments per condition.

FIG. 29 shows BAL total protein (FIG. 29A), BAL albumin (FIG. 29B), and lung tissue albumin (FIG. 29C) from male C57BL/6 mice after exposure to 2.5 mg/kg intratracheal lipopolysaccharide (LPS) and treatment one hour later with PBS vehicle, FTY720 (0.5 mg/kg), or 1S (doses labeled on the graph, milligram/kilogram) intraperitoneally, or S1P (0.026 mg/kg) via jugular vein injection simultaneous with LPS. n=4-5 animals per condition; *, p<0.05; **, p<0.01 compared to PBS vehicle treatment; and ***, p<0.001 and **, p<0.01 compared to PBS vehicle treatment.

FIG. 30A shows white blood cell (WBC) counts in BAL fluid collected from mice treated as described in FIG. 29; n=3-5 animals per condition. *, p<0.05, **, p<0.01, and ***, p<0.001 compared to PBS vehicle treatment. FIG. 30B shows lung tissue myeloperoxidase (MPO) activity as assayed in similarly treated mice; n=4-6 animals per condition. ** p<0.01 and ***, p<0.001 compared to PBS vehicle treatment.

FIG. 31A shows peripheral blood leukocyte counts in FTY720 analog- and LPS-treated mice. Mice received intratracheal LPS followed 1 h later by PBS, FTY720 (0.5 mg/ml), or 15 (doses labeled on the graph, milligram/kilogram) intraperitoneally. Blood was collected 18 h after LPS for total WBC (FIG. 31A) and lymphocytes (FIG. 31B) quantification. n=3-7 animals per condition. There are no statistical differences among any of the conditions shown.

### DETAILED DESCRIPTION OF THE INVENTION

The invention provides methods for treating or reducing the risk of developing acute lung injury that are not hampered by the limitations existing for conventional treatments. In particular, these methods were able to reverse or prevent the symptoms of acute lung injury manifested by increased vascular permeability or altered regulation of the ceramide/sphingolipid metabolic pathway. Advantageously, the inventive methods reduce increased vascular permeability that results from radiation treatment and can alleviate certain side effects, such as weight loss and hair loss, often associated with radiation treatment.

All molecular biology and DNA recombination techniques described herein are well known to one of ordinary skill in the art and further described in books such as Molecular Cloning: A Laboratory Manual (Sambrook, et al., 1989, Cold Spring Harbor Laboratory Press), which is incorporated herein by reference for any purposes. All references cited throughout the application are herein incorporated by reference in their entireties for any and all purposes.

As used herein, the singular forms “a”, “an” and “the” include plural referents unless the context clearly dictates otherwise.

As set forth herein, statins, a class of 3-hydroxy-3-methylglutaryl (HMG)-CoA reductase inhibitors, may serve as an RILI therapy since this class of drugs exerts potent pleiotropic anti-inflammatory, anti-thrombotic and immunomodulatory properties unrelated to lowering cholesterol. In addition, statins have been shown to be effective in rodent models of ALI. See Undas et al., 2002, Clin Lab 48:287-296; Jacobson et al., 2005, Am J Physiol Lung Cell Mol Physiol 288:L1026-1032. Simvastatin (marketed as Zocor® by Merck) was demonstrated in the instant application in a dose- and time-dependent murine RILI model as a potential therapeutic intervention for RILI.

Moreover, genome-wide lung expression profiling in an RILI subject as compared with control identified sphingolipid metabolic pathway genes as the target of gene dysregulation in RILI. The alternation of expression profile in the RILI subjects was significantly reduced in a RILI subject treated with simvastatin. Thus, it was unexpectedly discovered in the instant application that sphingolipid signaling components serve as important novel therapeutic targets and modulators of ALI, especially RILI.

The instant invention provides methods for treating or reducing ALI-associated dysregulation of the ceramide/sphingolipid metabolic pathway in a mammal by administering to a mammal an FTY720 analog or derivative thereof. FTY720 is a compound structurally similar to S1P with demonstrated barrier-enhancing activity, similar to S1P. See Camp et al. 2009, J Pharmacol Exp Ther 331:54. FTY720 and FTY are used interchangeably throughout this application, both referring to 2-amino-2-(4-octylphenethyl)propane-1,3-diol.

It was discovered by the inventors of the instant application that FTY720 analogs or derivatives, particularly FTY720 phosphonate, and more particularly the (S)-enantiomer of FTY720 phosphonate was more effective and better tolerated than FTY720 when administered at high concentrations and/or for longer period of time to a mammal suffering from ALI, especially radiation induced ALI. Thus, in accordance with the instant invention, a method is provided for treating or reducing the risk of acute lung injury by administering to a mammal an FTY720 derivative or analog.

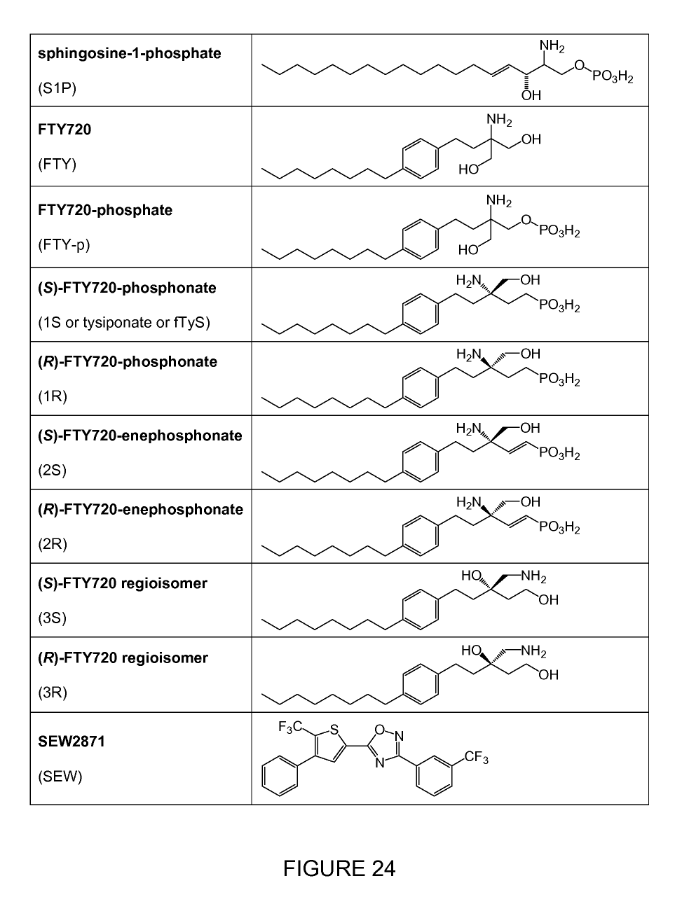

As used herein, the term “FTY720 derivative or analog” refers to a compound, natural or synthetic, that is structurally similar to FTY720 suitable for use in the instant invention but does not include FTY720 (2-amino-2-(4-octylphenethyl)propane-1,3-diol, see FIG. 24). In certain embodiments, the FTY720 analog or derivative is effective in treating or reducing the risks of developing ALI; in certain particular embodiments, the FTY720 analog or derivative is effective in treating or reducing the risks of developing radiation induced- or lung trauma-induced ALI. In certain embodiments, the FTY720 analog or derivative is effective in treating or reducing the risks of developing ALI in a mammal result from dysregulation of the ceramide/sphingolipid pathway. In certain other embodiments, the FTY720 analog or derivative is effective in reducing vascular leakage or vascular permeability in the lung, or reducing the risk of developing vascular leakage or increased vascular permeability in the lung of a mammal, reducing acute lung inflammation in a mammal, increasing alveolar cell integrity or increasing endothelial cell integrity in a mammal, reducing BAL protein levels or BAL cell count in a mammal, and/or reducing weight loss or hair loss associated with thoracic radiation therapy in a mammal FTY720 derivatives or analogs include, without limitation, the (R) or (S) enantiomer of FTY720-phosphonate, the (R) or (S) enantiomer of FTY720-enephosphonate, and the (R) or (S) enantiomer of FTY720 regioisomer (3-(aminomethyl)-5-(4-octylphenyl)pentane-1,3-diol), as shown in FIG. 24, or pharmaceutically acceptable salts thereof. In certain embodiments, the FTY720 analog or derivative is an FTY720-phosphonte, including the (R) and (S) enantiomers of FTY720-phosphonate, i.e., enantiomerically enriched or purified preparations of (R)- and (S)-3-amino-3-(hydroxymethyl)-5-(4-octylphenyl)pentylphosphonic acid, and the (R) and (S) enantiomer of FTY720-enephosphonate, i.e., enantiomerically enriched or purified preparations of (R)- and (S)-3-amino-3-(hydroxymethyl)-5-(4-octylphenyl)pent-1-enylphosphonic acid. In certain particular embodiments, the FTY720 analog or derivative is FTY720-phosphonte, including the (R) and (S) enantiomers of FTY720-phosphonate, i.e., enantiomerically enriched or purified preparations of (R)- or (S)-3-amino-3-(hydroxymethyl)-5-(4-octylphenyl)pentylphosphonic acid. In certain advantageous embodiments, the FTY720 analog or derivative is (S)-FTY720-phosphonte, also referred to as Tysiponate, Tysip, TyS, Tys, fTyS, or 1S throughout the instant application. In certain particular embodiments, the FTY720 analog or derivative does not include FTY720-phosphate (p-FTY720). The structure of (S)-FTY720-phosphonte (Tysiponate) is shown below:

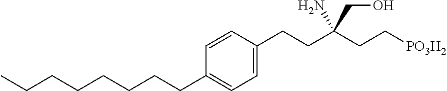

In additional embodiments, the invention provides methods of treating or reducing the risk of developing acute lung injury comprising the step of administering to a mammal in need thereof the compound SEW2871 (5-[4-phenyl-5-(trifluoromethyl)-2-thienyl]-3-[3-(trifluoromethyl)phenyl]-1,2,4-oxadiazole, see FIG. 24, obtainable from Cayman Chemical (Ann Arbor, Mich.)). SEW2871 is a selective S1P receptor agonist that is an immunosuppressant that does not induce bradycardia. Thus, in accordance with the instant invention, a method is provided for treating or reducing the risk of acute lung injury by administering to a mammal an FTY720 derivative or analog and/or SEW2871. In certain embodiments, the invention provides a method for treating or reducing the risk of acute lung injury by administering to a mammal an FTY720 derivative or analog, and further administering to the animal an effective amount of simvastatin and/or SEW 2871. In certain other embodiments, the invention provides a method for treating or reducing the risk of acute lung injury by administering to a mammal an FTY720 derivative or analog and SEW2871. In certain particular embodiments, the acute lung injury is RILI.

As used herein, the term “acute lung injury” or “ALI,” as opposed to chronic lung injury or condition, refers to a diffuse heterogeneous lung injury characterized by hypoxemia, non-cardiogenic pulmonary edema, low lung compliance, alveolar cell permeability, and widespread capillary leakage. The appearance of the symptoms of acute lung injury can vary depending on the cause of the injury—it takes hours or days in endotoxin-induced injury, while it can take weeks in radiation induced lung injury. ALI can be caused by stimulus of local or systemic inflammation, ionizing irradiation, infection, and exposure to bacterial endotoxin, sepsis, or trauma in the lung. Clinically, ALI can be diagnosed using one or more of the following parameters: bilateral pulmonary infiltrates on chest x-ray; pulmonary capillary wedge pressure <18 mmHg (2.4 kPa); and PaO₂/FiO₂<300 mmHg (40 kPa), where PaO₂ is the partial pressure of oxygen and FiO₂ is the fraction of inspired oxygen. The core pathology of ALI is disruption of the capillary-endothelial barrier, decreased endothelial integrity and increased pulmonary alveolar permeability. Disruption of endothelial barrier can result in protein-rich fluid leaking out of the capillaries. Acute lung injury as described herein can lead to chronic lung conditions, which is generally characterized by lung tissue remodeling and fibrosis.

There are two types of alveolar epithelial cells: Type 1 pneumocytes consists of 90% of the surface area of the lung that are more susceptible to damage compared with Type 2 pneumocytes, which are more resistant to damage, produce surfactant, transport ions and proliferate and differentiate into the Type 1 cells. Injury to epithelial cells impairs the lung's ability to pump fluid out of the airspaces. Fluid build-up in the airspaces, loss of surfactant, microvascular thrombosis and disorganized repair (which can lead to fibrosis) can result in reduced resting lung volumes (decreased compliance), increased ventilation-perfusion mismatch, and increased effort required for breathing. In addition, lymphatic drainage of the lung appears to be reduced, which further contributes to the buildup of extravascular fluid. Severe ALI can lead to acute respiratory distress syndrome (ARDS).

ALI can be caused by a variety of means, such as ionizing radiation. “Radiation-induced lung injury” or “RILI” is a general term for injuries sustained by the lungs as a result of exposure to ionizing radiation, which most commonly occurs as a result of radiation therapy of thoracic cancer. Such damage includes early (acute) inflammatory damage (radiation pneumonitis) and later complications of chronic scarring (radiation fibrosis). RILI is a particular subset of ALI, with a unique patient population (most commonly patients receiving radiation therapy), unique nature of injury (radiation-induced injury), and a slight delay of onset of disease (weeks vs. hours/days as compared with LPS-induced ALI). Clinically, RILI may be characterized by loss of epithelial cells, edema, inflammation, and occlusions of airways, air sacs, and blood vessels. The lungs are the most radiosensitive organ, and radiation pneumonitis can lead to pulmonary insufficiency and death (100% after exposure to 50 Gy of radiation) in a few months. Injuries most suitable for treatment of the instant application include inflammatory damage (radiation pneumonitis) manifested by increased pulmonary permeability.

In certain advantageous embodiments, the invention provides methods of preventing or reducing the risk of developing radiation-induced lung injury in a patient scheduled for radiation therapy comprising the step of administering an FTY720 analog or derivative or SEW2871 to the patient before, concurrently with or after radiation therapy. In particular embodiments, the FTY720 analog or derivative or SEW2871 is administered to the patient before, concurrently with, of after radiation therapy. In certain particular embodiments, the FTY720 analog or derivative or SEW2871 is administered to the patient before radiation therapy.

As used herein, the term “radiation therapy of thoracic cancer” or “thoracic radiation therapy” refers to radiation treatment of thoracic cancer. Non-limiting examples of thoracic cancer include lung cancer, esophagus cancer, trachea cancer and cancer of the chest wall. Frequently, the lung tissue is directly damaged due to the radiotherapy of lung cancer, or indirectly damaged during radiotherapy of cancers of other tissues in the thoracic cavity. In certain advantageous embodiments, methods are provided for reducing weight loss or hair loss associated with thoracic radiation therapy in a mammal in need thereof comprising the step of administering to a mammal in need thereof an FTY720 derivative or analog.

As used herein, the term “concurrently with” refers to administering to a patient in need for radiation therapy an FTY720 analog or derivative or SEW2871 during the period when the patient is receiving radiation treatment, but is not limited to the exact time when the patient is undergoing radiation treatment. For example, when a patient is scheduled for a week-long session of radiation treatment, the patient may receive an FTY720 analog or derivative or SEW2871 of the invention during the same week; and thus, the FTY720 analog or derivative or SEW2871 is administered concurrently with the radiation therapy. On the other hand, the administration of the FTY720 analog or derivative or SEW2871 prior to the starting of, or after the completion of, the week-long radiation therapy is administered before or after the radiation therapy, respectively.

As used herein, the term “ceramide/sphingolipid metabolic pathway” refers to the mammalian enzymatic pathways relating to the regulation, synthesis and elimination of ceramide, S1P and other metabolites of sphingolipid. Non-limiting examples of the components of the ceramide/sphingolipid metabolic pathway include S1P, DHS1P, S1P receptors, sphingosine kinase, S1P phosphatase, sphingosine lyase, ceramide, ceramidase, and sphingomyelinase. The term “dysregulation of the ceramide/sphingolipid metabolic pathway” as used herein refers to for example the changes of the levels of one or more components in the ceramide/sphingolipid metabolic pathway, or the changes of the ratio of multiple components in the ceramide/sphingolipid metabolic pathway, in a sample obtained from a mammal as compared to a control sample. In certain particular embodiments, the sample is a sample from the lung. The control sample can be a sample from a control subject or a sample from the same mammal before the mammal has developed ALI. In certain particular embodiments, methods are provided for treating or reducing the risk of developing ALI as a result of the dysregulation of the ceramide/sphingolipid metabolic pathway in the lung of a mammal comprising the step of comprising the step of administering to a mammal in need thereof an FTY720 derivative or analog or SEW 2871 in an amount capable of reversing dysregulation of the ceramide/sphingolipid metabolic pathway.

In certain embodiments, the dysfunction of the ceramide/sphingolipid metabolic pathway is indicated by the reduced combined levels of S1P and DHS1P in a sample from the lung of a test mammal as compared to a control sample from a lung. In certain other embodiments, the dysfunction of the ceramide/sphingolipid metabolic pathway is indicated by the increased levels of ceramide in a sample from the lung of a test mammal as compared to a control sample from a lung. In certain particular embodiments, the ceramide molecular species measured include N-acylated C18-sphingosine with strait-chain fatty acids of C14 to C28 and optionally may contain one double bond. The average content of total ceramide in the BAL in a control mouse without receiving radiation is 92 pmol/ml. The amount peaked at 2780 pmol/ml (at 4 weeks after radiation). At 6 weeks after radiation, total ceramide level was 493 pmol/ml and further decreases with time. In certain embodiments, the combined levels of S1P and DHS1P and the levels of ceramide are measured in a sample taken at or about four-six weeks after the mammal is exposed to radiation. In certain particular embodiments, the sample is taken at or about four weeks after the mammal is exposed to radiation; and in other particular embodiments, the sample is taken at or about six weeks after the mammal is exposed to radiation.

The interconvertible sphingolipid metabolites ceramide, sphingosine, and S1P not only differ in their physical and signaling properties, but also have counteracting effects. For example, S1P is able to counteract ceramide-mediated apoptosis, and the balance between these two metabolites, or the balance between the combined levels of S1P and DHS1P on the one hand, and ceramide on the other, influences growth and survival in eukaryotic cells. The term sphingolipid “rheostat” reflects the adjustable nature of this balance. The changes in the sphingolipid rheostat may involve changes in the absolute levels of the bioactive sphingolipid metabolites and temporal or local differences in the relative ratios of these metabolites, providing a “built-in” inducible regulatory switch for controlling cellular responsiveness.

ALI can also be induced by bacterial endotoxin. The term “endotoxin” refers to a toxin produced by Gram-negative or Gram-positive bacteria. More specifically, an endotoxin is a structural molecule of a bacterium that is recognized by the immune system. Prototypical examples of endotoxin are lipopolysaccharide (LPS) or lipooligosaccharide (LOS) found in the outer membrane of various Gram-negative bacteria, including Escherichia coli, and are an important component of their ability to cause disease. LPS consists of a polysaccharide (sugar) chain and a lipid moiety, known as lipid A, which is responsible for the toxic effects. The polysaccharide chain is highly variable amongst different bacteria. Endotoxins are in large part responsible for the dramatic clinical manifestations of infections with pathogenic Gram-negative bacteria, such as Neisseria meningitidis, the pathogens that causes meningococcal disease, including meningococcemia, Waterhouse-Friderichsen syndrome and meningitis. Other endotoxins include the delta endotoxin of Bacillus thuringiensis, which makes crystal-like inclusion bodies next to the endospore inside the bacteria. In addition, Listeria monocytogenes may produce an “endotoxin-like” substance.

ALI can also be caused by trauma. “Trauma” refers to a body wound or shock produced by sudden physical injury in the lung, as from violence or accident. The effects of disruption of the endothelial barrier as a result of physical injury can be alleviated by the methods of the instant invention.

In certain aspects, the invention provides methods for reducing vascular leakage or vascular permeability in the lung. Diseases that present the symptoms of increased vascular leakage or increased vascular permeability in the lung can be characterized generally as vascular permeability disorders in the lung, including ALI, respiratory distress syndrome, and ventilator-induced lung injury (VILI). The increased vascular leakage and permeability of these vascular disordered in the lung can be alleviated by the instant invention. Permeability of pulmonary endothelial cells and pulmonary alveolar cells can be assessed by the protein levels and cell count in the bronchoalveolar lavage (BAL) of a mammal, wherein higher protein levels or cell count in the BAL as compared to control indicates increased pulmonary endothelial and epithelial permeability. Accordingly, other aspects of the invention provide methods for decreasing BAL protein levels or BAL cell count in a mammal comprising the step of administering to a mammal in need thereof an effective amount of an FTY720 derivative or analog or SEW2871.

As used herein, the term “effective amount” or a “therapeutically effective amount” of a compound refers to an amount sufficient to achieve the stated desired result, for example, treating or reducing the risk of developing acute lung injury, particularly radiation-induced lung injury, or acute lung inflammation. Additional desired results also include reducing vascular leakage or vascular permeability in the lung, increasing alveolar cell integrity or increasing endothelial cell integrity in the lung, reducing BAL protein levels or BAL cell count, and reducing weight loss or hair loss associated with radiation therapy. The amount of a compound which constitutes an “effective amount” or “therapeutically effective amount” will vary depending on the compound, the disorder and its severity, and the age of the subject to be treated, but can be determined routinely by one of ordinary skill in the art.

“Treating” or “treatment” as used herein covers the treatment of a disease or disorder described herein, in a subject, preferably a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder.

“Preventing” or “reducing the risk of developing” a disease or condition as used herein refers to (i) inhibiting the onset of a disease or a condition in a subject or patient who may be at risk of or predisposed to developing the disease or condition; and/or (ii) slowing the onset of the pathology or symptom of a disease or condition in a subject or patient who may be at risk of or predisposed to developing the disease or condition. For example, pretreatment of a patient the pharmaceutical composition comprising an FTY analog or derivative or SEW 2871 before radiation therapy can reduce the risk of the patient in developing radiation-induced lung injury associated with the radiation therapy.

In certain embodiments of any of the aspects of the invention, a “subject” or “patient” refers to a mammal in need of the treatment of the instant invention. In certain particular embodiments, the mammal is a human.

In a further aspect, the invention provides a pharmaceutical composition comprising a therapeutic effective amount of an FTY720 analog or derivative or SEW 2871 and a pharmaceutically acceptable diluent, carrier or excipient. In certain particular embodiments, the FTY720 analog or derivative is the (R)- or (S)-enantiomer of FTY720 phosphonate, the (R)- or (S)-enantiomer of FTY720-enephosphonate, or the (R)- or (S)-enantiomer of FTY720 regioisomer. In particular embodiments, the pharmaceutical compositions comprises an effective amount of the (S)-enantiomer of FTY720 phosphonate. In certain particular embodiments, dosage ranges suitable for use in the instant invention are from about 0.01 mg/kg to about 7.0 mg/kg, and in other particular embodiments, from about 0.01 mg/kg to about 1 mg/kg, from about 0.01 mg/kg to about 5 mg/kg, from about 0.05 mg/kg to about 3 mg/kg, from about 0.5 mg/kg to about 2 mg/kg, from about 0.5 mg/kg to about 3 mg/kg, from about 0.5 mg/kg to about 5 mg/kg, from about 1 mg/kg to about 4 mg/kg, and from about 1 mg/kg to about 5 mg/kg.

In another aspect, the invention provides a pharmaceutical dosage form comprising an effective amount of an FTY720 analog or derivative or SEW 2871. As used herein, the term “dosage form” refers to the physical form of a dose of a therapeutic compound, such as a capsule or table, intended to be administered to a patient. The term “dosage unit” as used herein refers to the amount of the therapeutic compounds to be administered to a patient in a single dose. In certain embodiments, the dosage unit suitable for use in the instant application are (assuming the weight of an average patient being 70 kg) 0.7 mg/dosage unit-about 500 mg/dosage unit, and in certain other embodiments, 0.7 mg/dosage unit to about 70 mg/dosage unit, from about 0.7 mg/dosage unit to about 350 mg dosage/unit, from about 3.5 mg/dosage unit to about 210 mg/dosage unit, from about 35 mg/dosage unit to about 140 mg/dosage unit, from about 35 mg/dosage unit to about 210 mg/dosage unit, from about 35 mg/dosage unit to about 350 mg/dosage unit, from about 70 mg/dosage unit to about 280 mg/dosage unit, and from about 70 mg/dosage unit to about 350 mg/dosage unit. It is within the knowledge of a skilled artisan or physician to determine the effective dosages ranges or dosage forms based on several factors such as the age and disease condition of a patient. In certain particular embodiments, the FTY720 analog or derivative is the (R)- or (S)-enantiomer of FTY720 phosphonate, the (R)- or (S)-enantiomer of FTY720-enephosphonate, or the (R)- or (S)-enantiomer of FTY720 regioisomer. In particular embodiments, the pharmaceutical compositions comprises an effective amount of the (S)-enantiomer of FTY720 phosphonate.

“Pharmaceutically acceptable” as used herein refers to those compounds, materials, compositions, and/or dosage forms which are, within the scope of sound medical judgment, suitable for contact with the tissues of human beings and animals without excessive toxicity, irritation, allergic response, or other problems or complications commensurate with a reasonable benefit/risk ratio or which have otherwise been approved by the United States Food and Drug Administration as being acceptable for use in humans or domestic animals.

“Pharmaceutically acceptable salt” refers to both acid and base addition salts.

The pharmaceutical compositions of the invention may contain formulation materials for modifying, maintaining, or preserving, in a manner that does not hinder the physiological function and viability of the analog or agonist, for example, pH, osmolarity, viscosity, clarity, color, isotonicity, odor, sterility, stability, rate of dissolution or release, adsorption, or penetration of the composition. Suitable formulation materials include, but are not limited to, amino acids (such as glycine, glutamine, asparagine, arginine, or lysine), antimicrobial compounds, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, betacyclodextrin, or hydroxypropyl-beta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as polysorbate 20 or polysorbate 80; triton; trimethamine; lecithin; cholesterol or tyloxapal), stability enhancing agents (such as sucrose or sorbitol), tonicity enhancing agents (such as alkali metal halides—preferably sodium or potassium chloride—or mannitol sorbitol), delivery vehicles, diluents, excipients and/or pharmaceutical adjuvants. See REMINGTON'S PHARMACEUTICAL SCIENCES (18th Ed., A. R. Gennaro, ed., Mack Publishing Company 1990).

Optimal pharmaceutical compositions can be determined by one skilled in the art depending upon, for example, the intended route of administration, delivery format and desired dosage. See, for example, REMINGTON'S PHARMACEUTICAL SCIENCES, Id. Such compositions may influence the physical state, stability, rate of in vivo release and rate of in vivo clearance of the antibodies of the invention.

Administration routes for the pharmaceutical compositions of the invention include orally, through injection by intravenous, intraperitoneal, intramuscular, intratracheal, intravascular, intraarterial, intraportal, or intralesional routes; by sustained release systems or by implantation devices. The pharmaceutical compositions may be administered by bolus injection or continuously by infusion, or by implantation device. The pharmaceutical composition also can be administered locally via implantation of a membrane, sponge or another appropriate material onto which the desired molecule has been absorbed or encapsulated. Where an implantation device is used, the device may be implanted into any suitable tissue or organ, and delivery of the desired molecule may be via diffusion, timed-release bolus, or continuous administration.

The Examples that follow are illustrative of specific embodiments of the invention, and various uses thereof. They are set forth for explanatory purposes only, and are not to be taken as limiting the invention.

### EXAMPLES

### Example 1

### Characterization of a Murine Model of RILI

To characterize a murine model of RILI, female C57BL/6 mice were exposed to a single dose of whole thoracic radiation (18-25Gy) and indices of lung inflammation and vascular permeability assessed at intervals of 4, 6, 8, and 12 wks.

Eight to 10-week-old C57BL/6J mice (Jackson Labs, Bar Harbor, Me.) were used for all studies in accordance with the University of Chicago Institutional Animal Care & Use Committee guidelines. Mice were anesthesized with ketamine (100 mg/kg) and acepromazine (1.5 mg/kg) prior to single dose irradiation.

Bronchoalveolar lavage (BAL) was performed as follows (see also Moitra et al., Transl Res 2008; 151:141-153). At the termination of each experiment, animals were euthanized by exsanguination under anesthesia in accordance with institutional guidelines. Both lungs were lavaged with 1.0 mL of Hank's buffered saline solution with the fluid allowed to equilibrate for 10 s before withdrawing. The pulmonary vasculature was perfused clear via the pulmonary artery with sterile phosphate-buffered saline (PBS). Both lungs were excised, weighed, and snap-frozen in liquid nitrogen for subsequent analysis. The BAL fluid collected was centrifuged at 500×g for 20 min at 4° C., and the supernatant was removed and recentrifuged at 12,000×g before snap-freezing. Cell pellets were resuspended in 0.5 mL of red blood cell lysis buffer (ACK Lysing Buffer; BioSource International, Camarillo, Calif.) for 20 min and then repelleted by centrifugation at 2500 rpm for 20 min at 4° C. The supernatant was decanted, and the cell pellet was resuspended in 0.2 mL of PBS for cellular analysis using a standard hemacytometer technique. A total of 300 BAL cells per slide were counted for cell differentials using a Diff-Quick-stained kit (Baxter Diagnostics, McGaw Park, Ill.). BAL protein concentrations were determined by a colorimetric BCA assay. Albumin concentrations in the BAL at 1:1000 and 1:100 dilutions, respectively, were quantitated by ELISA (Bethyl Labs, Montgomery, Tex.). To measure cytokines and chemokines, the BAL fluid was assayed with a Bioplex mouse cytokine kit (Bio-Rad, Hercules, Calif.) in accordance with the manufacturer's instructions.

Evans blue dye (EBD) (5 mg/kg) extravasation into lung tissue was performed as follows (see also Moitra et al., Transl Res 2007; 150:253-265). Tetrasodium salt of EBD (tetrasodium 4-amino-6-[4-[4-(8-amino-1-hydroxy-5,7-disulfonato-naphthalen-2-yl)diazenyl-3-methyl-phenyl]-2-methyl-phenyl] diazenyl-5-hydroxy-naphthalene-1,3-disulfonate; MW 960.8), and bovine serum albumin (BSA) (fraction V, low-endotoxin; MW 66,000) were obtained from Sigma Chemical Co. (St. Louis, Mo.). All other chemicals were analytical or cell-culture grade and were obtained from various commercial sources. Evans Blue dye conjugated to albumin (EBA) was prepared by dissolving EBD to a concentration of 0.5% (5 mg/mL or 5.2 mM) in phosphate-buffered saline (PBS) (Ca²⁺-Mg²⁺ free; Invitrogen Corporation, Carlsbad, Calif.). To this solution, BSA was added to a final concentration of 4% (40 mg/mL or 0.6 mM), mixed well by stirring with a magnetic bar, allowed to stand for 30 min, and then sterile-filtered through a 0.22-μm syringe filter. The conjugate was stored in small aliquots at −80° C. To prevent cross-contamination, each aliquot was used only once in one animal and then discarded. 4, 6, 8, or 12 weeks after irradiation, the animals were injected with EBA via the jugular vein. At the end of exposure, the pulmonary circulation was flushed and lungs were harvested. Formamide extracts of lungs were prepared, with the initial modification that the homogenates were centrifuged at 12,000×g. Centrifugation at this relative centrifugal force (RCF) produced a compact pellet that allowed accurate measurement of the recovered volumes. Accurate estimation of such volumes is critical for calculating the final tissue dilution factor. Lungs, kidneys, and hearts from mice that were not injected with EBA were similarly extracted, to calculate the tissue-specific correction factors. The centrifuged supernatants were measured at 620 and 740 nm in a spectrophotometer capable of reading in 2 wavelengths simultaneously (UV-1201; Shimadzu, Columbia, Md.) or a 96-well plate reader fitted with 620-nm and 750-nm bandpass filters (cutoff±20 nm; ThermoMax; Molecular Devices, Sunnyvale, Calif.), against a blank (50% formamide in PBS). A standard curve of EBA was prepared in the same solution (linear in the range 0.12 to 31.25 μg/mL; P<0.01 by F-test). Linear regression equations between absorbance at 740 nm (X) and 620 nm (Y) in tissue extracts of animals untreated with EBA were considered to be the tissue-specific correction factors. The observed absorbance of the control and radiation-treated samples at 620 and 740 nm was then normalized using this factor, and the corresponding values were read by inverse prediction of the regression equation describing the standard curve. The EB concentration read in mg/mL was converted to μg/g wet weight of lungs using the dilution factor of the original homogenate as described above.

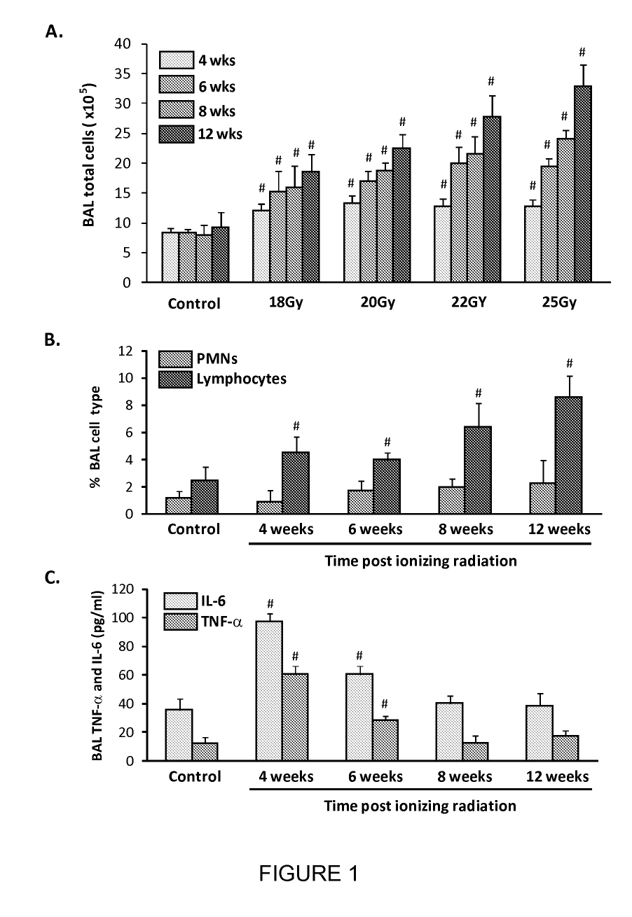

Murine RILI evolved in a dose- and time-dependent fashion (over 12 wks) with increased vascular leak and leukocyte infiltration. As shown in FIG. 1A, thoracic radiation (18-25 Gy) is associated with dose- and time-dependent effects on murine BAL total cell counts. BAL cellularity was not altered prior to 6 wks whereas significant increases in BAL cells was observed at all radiation levels by 12 wks post-irradiation (n=10 animals/experimental group, #, p<0.01 compared to control). Irradiated mice demonstrated significant time-dependent increases in levels of BAL total cell counts (FIG. 1A) with alveolar macrophages representing the dominant BAL cell type (>85%, with this percentage unchanged with radiation) although the percentage of BAL lymphocytes significantly increased at 8 and 12 wks post-irradiation (FIG. 1B). Macrophages represent the dominant cell type (>80%) in each BAL sample with marked expansion of this cell population in BAL at 12 wks (25 Gy, data not shown). A significant increase in the percentage of BAL lymphocytes (10-20%, #, p<0.01 compared to controls) was noted at 4 to 12 wks after irradiation (25 Gy) whereas BAL neutrophil counts (PMNs) were not significantly increased in irradiated mice at any time point (FIG. 1B). Time-dependent effects of radiation are shown in FIG. 1C with respect to BAL levels of IL-6 and TNF-α in murine RILI. Mice received a single dose of radiation (25 Gy) or mock irradiation to the thorax and were longitudinally followed (4, 6, 8, and 12 wks). Significant increases in IL-6 and TNF-α relative to controls were observed at 4 and 6 wks, suggesting an early role of these cytokines in barrier dysfunction (n=4 animals/experimental group, #, p<0.01). Companion studies identified significant time-dependent increases in inflammatory markers after radiation including IL-6 and TNF-α measured in BAL fluid (FIG. 1C). In contrast to BAL cell counts, these studies revealed an early increase in BAL cytokines at 4 wks and progressive decline and returning to basal levels by 8 wks.

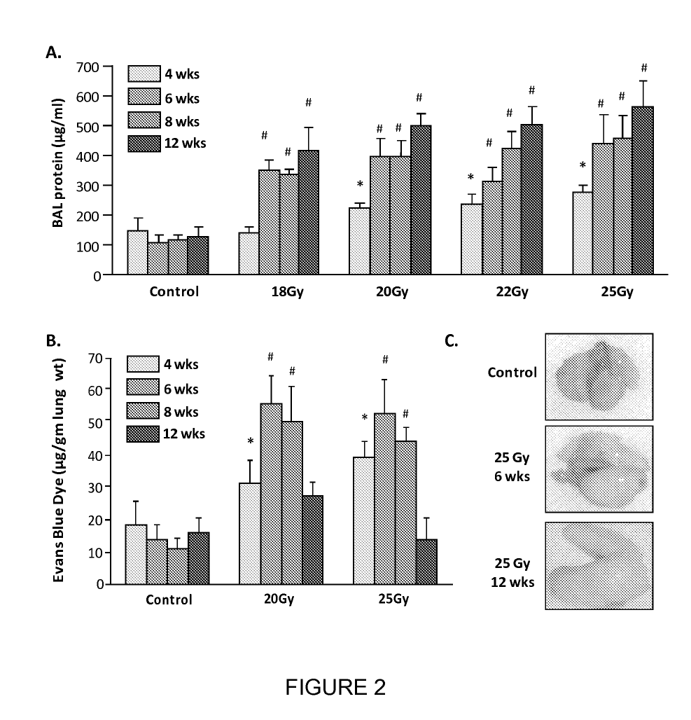

Irradiated mice demonstrated significant dose- and time-dependent increases in alveolar permeability with progressive increases in BAL protein (FIG. 2A) beginning at 2 wks and sustained throughout the 12-week period. Irradiated mice exhibited significantly increased BAL protein at 6 wks, with values that peaked at 12 wks (n=7 animals/experimental group, * p<0.05 and #, p<0.01 compared to respective controls). Radiation-mediated (20 and 25 Gy) increases in the extravasation of intravenously delivered EBD into the lung interstitium, used as a surrogate marker for vascular permeability (Moitra et al., Transl Res 2007, 150:253-265), peaked at 6 to 8 wks post-irradiation (n=4, * p<0.05, #, p<0.01) and returned to control levels by 12 wks (FIGS. 2B and 2C) suggesting differential susceptibilities of alveolar and lung vascular barriers to thoracic radiation. Overall, these results demonstrated successful establishment of a murine model of RILI, which was shown to evolve in a dose- and time-dependent fashion over 12 wks with increased vascular permeability and lung inflammation.

### Example 2

### Increased Histologic Inflammation and Lung Nitrotyrosine Expression in Murine RILI

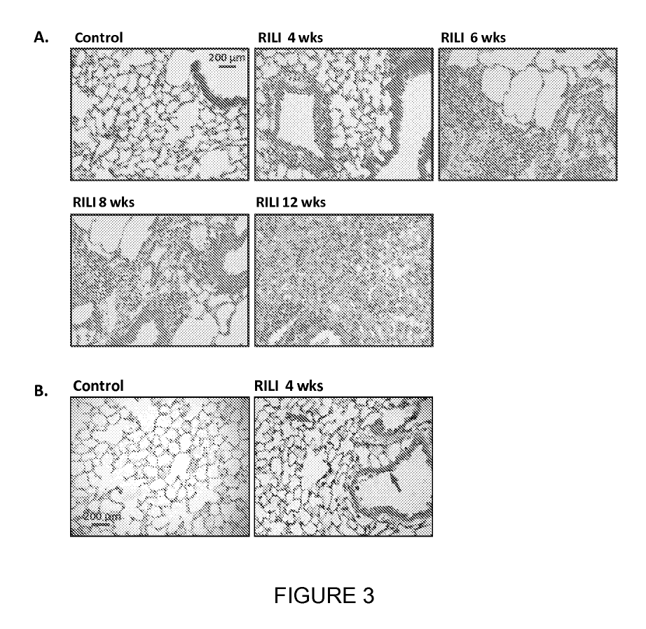

To investigate mechanisms involved in RILI, nitrotyrosine expression, a marker of peroxynitrite generation and RILI-induced oxidant injury (Giaid et al., Am J Clin Oncol 2003, 26:e67-72) was assessed in the mouse model of RILI. Lungs from mice at 6 wks post-irradiation exhibited modest alveolar flooding and inflammatory foci in scattered areas with perivascular clustering of inflammatory cells prominent at 8 and 12 wks. Compared to control lungs, hematoxylin and eosin staining of murine lung sections showed considerable damage to Type I pneumocytes at 4 wks post-irradiation (25 Gy) without visible edema and inflammation (FIG. 3A). Nitrotyrosine expression was confined to lung epithelium, endothelium and alveolar macrophages and, similar to BAL cytokine levels, peaked at 4 wks (FIG. 3B) but then declined to control levels 12 wks post-irradiation despite increasing evidence of histologic injury (data not shown).

The decline in nitrotyrosine expression and BAL cytokine levels to control levels by 12 wks post-irradiation suggests that these elements contribute to early histological injury but not to more delayed injury. In this regard, unlike other murine RILI models (Ostrau et al., 2009, Radiother Oncol 92:492-499; Williams et al., 2004, Radiat Res 161:560-567; Iwakawa et al., 2004, J Radiat Res (Tokyo) 45:423-433), these results suggest discordance between alveolar epithelial barrier function and vascular leakage, hallmarks of inflammatory lung injury and a phenotypic consequence of RILI (Gross, 1980, J Lab Clin Med 95:19-31), which may be linked to differential susceptibility of type I pneumocytes and vascular endothelium to ionizing radiation. Alveolar injury and barrier dysfunction were sustained at lower levels of radiation and remained progressive over the 12 wks of RILI, whereas radiation-induced vascular leakage occurred only with the highest radiation dose and completely resolved by 12 wks. Epithelial and endothelial injury and barrier dysfunction were facilitated by the increased levels of oxidative and nitrosative stress induced by direct ionizing radiation (Giaid et al., 2003, Am J Clin Oncol 26:e67-72; Hallahan et al., 1998, Cancer Res 58:5484-5488; Zhao and Robbins, 2009, Curr Med Chem 16:130-143).

### Example 3

### Protective Effects of Simvastatin in a Preclinical Model of RILI

As lung inflammation and sustained alveolar barrier dysfunction are prominent features in murine RILI pathobiology, the effects of simvastatin, an effective anti-inflammatory and lung edema-reducing pharmacological agent (Jacobson et al., 2005, Am J Physiol Lung Cell Mol Physiol 288:L1026-1032) were assessed on murine RILI.

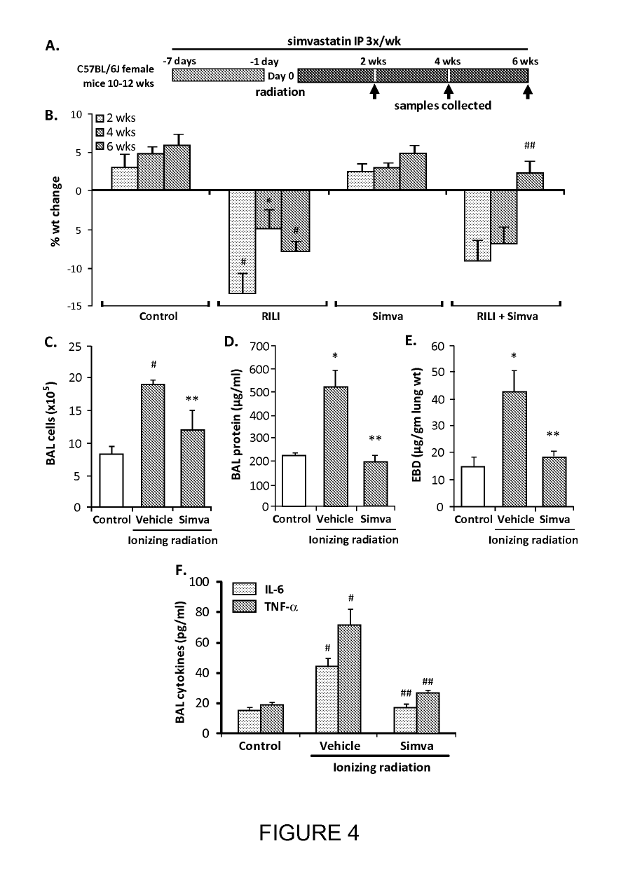

Eight to 10-week-old C57BL/6J mice (Jackson Labs, Bar Harbor, Me.) were used for all studies in accordance with the University of Chicago Institutional Animal Care & Use Committee guidelines. Mice were anesthesized with ketamine (100 mg/kg) and acepromazine (1.5 mg/kg) prior to single dose irradiation. Mice were treated with 10 mg/kg simvastatin (Sigma, St Louis) via intraperitoneal injection 3×/week beginning 1 week prior to irradiation and continuing up to 6 weeks post-irradiation. (FIG. 4A). Bronchoalveolar lavage (BAL) was performed as described above. BAL protein concentrations were determined by a colorimetric BCA assay. Albumin concentrations in the BAL at 1:1000 and 1:100 dilutions, respectively, were quantitated by ELISA (Bethyl Labs, Montgomery, Tex.). To measure cytokines and chemokines, the BAL fluid was assayed with a Bioplex mouse cytokine kit (Bio-Rad, Hercules, Calif.) in accordance with the manufacturer's instructions. Evans blue dye (EBD) (5 mg/kg) extravasation into lung tissue was performed as described above. The EBD concentration is expressed as μg/g wet weight of lung tissue. For lung histology and immunohistochemistry studies, lungs were inflated to 30 cm H₂O with 10% formalin for histological evaluation by hematoxylin and eosin staining (Hong et al., 2008, Am J Respir Grit Care Med 178:605-617; Meyer et al., 2009, Faseb J 23:1325-1337).

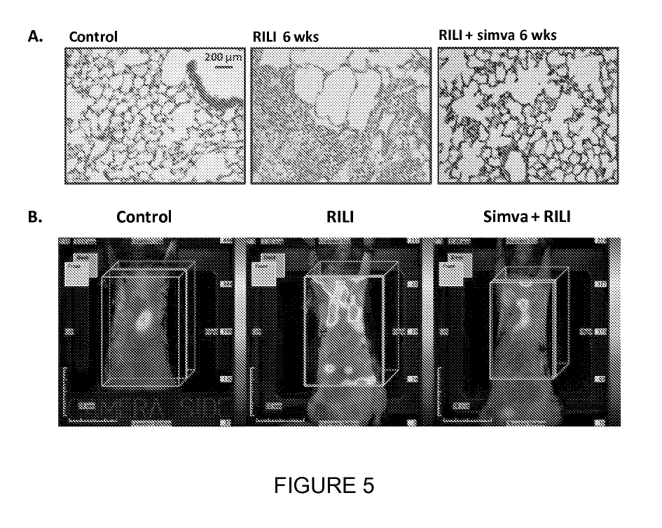

Simvastatin significantly attenuated radiation-induced weight loss (FIG. 4B); simvastatin treatment significantly increased body weight at 6 wks compared to radiated controls (n=5 animals/experimental group; ##, p<0.01 compared to RILI alone) while radiation exposure alone produced significant weight loss at 2, 4, and 6 wks (*p<0.05 and #, p<0.01 compared to controls). Simvastatin also attenuated radiation-induced increases in BAL inflammatory cells (FIG. 4C), and both alveolar and lung vascular leak as assessed by BAL protein levels (FIG. 4D) and EBD extravasation (FIG. 4E) at 6 wks (n=5 animals/experimental group, * p<0.05 and #, p<0.01 compared to controls; ** p<0.05 compared to RILI alone). At 4 wks post-irradiation, simvastatin treatment was associated with a significant reduction in BAL pro-inflammatory cytokines, TNF-α and IL-6 (n=5 animals/experimental group, #, p<0.01 compared to controls and ##, p<0.01 compared to radiation alone) (FIG. 4F). In addition, pro-inflammatory cytokines levels were significantly reduced in the BAL fluid of simvastatin-treated animals compared to radiated controls (FIG. 4F). These findings were corroborated by findings on lung histology as alterations in lung architecture with edema formation and lung inflammation with considerable Type I pneumocytes damage were markedly reduced in the lungs of simvastatin-treated animals (FIG. 5A). Significant attenuation of murine RILI and inflammatory cell infiltration in lungs from animals treated with simvastatin was apparent at 6 wks post-irradiation.

Lung imaging studies were also performed with assessment of RILI-induced extravasation of an intravascular probe. Simvastatin-treated RILI mice (25 Gy) and control mice exposed to radiation alone without simvastatin (6 wks post-irradiation) were injected (i.v.) with an intravascular non-targeted blood pool probe (Angiosense⁶⁸⁰) and imaged (ViSen FMT imaging) at 24 hrs post-angiosense injection allowing the extent of lung leakiness and injury to be quantified as fluorescent intensity. Control mice demonstrated dye retention in the vasculature (box) whereas untreated RILI mice exhibited vascular leakage by the extravasation of dye into the lung parenchyma. Simvastatin treatment of RILI mice significantly decreased dye extravasation. These experiments further confirmed the protective effects of simvastatin evidenced by an attenuation of probe signal throughout the lungs of radiation mice treated with simvastatin compared to radiated controls.

Overall, simvastatin markedly reduced multiple RILI inflammatory indices including leukocyte infiltration and lung permeability, consistent with prior studies in rodent models of LPS-induced acute lung injury (Jacobson et al., 2005, Am JPhysiol Lung Cell Mol Physiol 288:L1026-1032), ischemia reperfusion injury (Moreno-Vinasco et al., 2008, Journal of Organ Dysfunction 4: 106-114) and pulmonary hypertension (Girgis et al., 2003, Am JPhysiol Heart Circ Physiol 285:H938-945). While the protective effects of statins in animal models of radiation injury have previously been investigated (Ostrau et al., 2009 Radiother Oncol 92:492-499; Williams et al., 2004, Radiat Res 161:560-567) the studies presented herein are the first to characterize the lung vascular protective effects of statins in RILI.

### Example 4

### Deregulation of Gene Expression and Biological Pathways Induced by RILI and Attenuated by Simvastatin

Expression profiling of murine lung tissues was used to investigate differential lung gene expression in response to simvastatin treatment. Total RNA was extracted from lungs using TRIzol reagent (Invitrogen, Carlsbad, Calif.) and RNeasy kit (Qiagen, Valencia, Calif.) (Hong et al., 2008, Am J Respir Crit. Care Med 178:605-617; Meyer et al., 2009, Faseb J 23:1325-1337) and was used to synthesize double-stranded cDNA using the One-Cycle DNA Synthesis Kit (Affymetrix, Santa Clara, Calif.). Biotin-labeled antisense cRNA was then generated and hybridized to the Affymetrix Mouse Genome 430 2.0 Array as described in the Affymetrix GeneChip protocol.

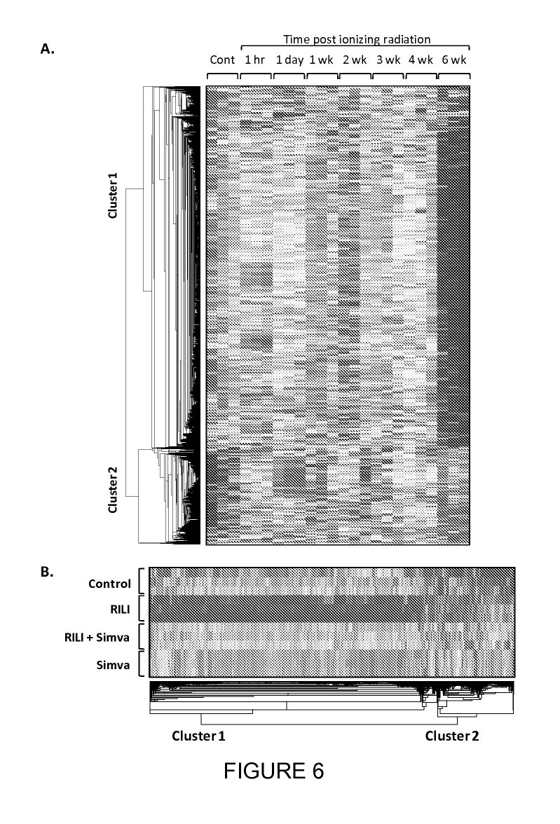

Oligonucleotide arrays were normalized and processed using Bioconductor “GCRMA” package. To identify differentially expressed genes, two group comparisons were conducted using Significance Analysis of Microarrays (Tusher et al., 2001, Proc. Natl. Acad. Sci. USA 98:5116-5121). Data have been submitted to the Gene Expression Omnibus repository of the National Center for Biotechnology Information and have been published (GSE14431). Expression profiles revealed robust radiation-induced differential lung gene expression which was reversed by simvastatin (Table 1). The clustered heat map of radiation-induced dysregulated genes across all time points revealed that only a small sub-group of genes were dysregulated in the early post-radiation phase (1 hour and one day) whereas the majority of gene dysregulation occurred as unique late phase changes (6 wks post-irradiation, FIG. 6A). Simvastatin normalized radiation-mediated transcriptional suppression (FIG. 6B) which was evidenced by the greater number of radiation-induced down-regulated genes (2547 down-regulated genes) compared to up-regulated genes (677 up-regulated genes, Table 1). These findings were validated by Gene Ontology analysis which revealed that five of the seven major radiation-inhibited biological processes were related to transcriptional regulation or processing (transcription, mRNA processing, DNA-dependent regulation of transcription, chromatin modification and RNA splicing) and were reversed by simvastatin (Table 2).

TABLE 1
Gene filtering criteria and results
by significant analysis of microarray

| Gene | 2-group | | | | Probe | | |
|---|---|---|---|---|---|---|---|
| List* | comparison | Delta | FDR % | Fold | sets | Up | Down |
| 1 | Radiation vs. | 15 | 3.8 | 2.0 | 3224 | 677 | 2547 |
| | Control | | | | | | |
| 2 | Radiation-Simva | 15 | 4.5 | 2.0 | 3037 | 2560 | 477 |
| | vs. Radiation | | | | | | |
| 3 | Radiation- Simva | 15 | 3.0 | 2.0 | 933 | 461 | 472 |
| | vs. Simva | | | | | | |

Gene list 1, 2 and 3 are the microarray result of 6 wks observation (GEO accession number GSE14431). The full lists of genes can be found in website http://phenos.bsd.uchicago.edu/publication/Radiation-Simvastatin.

TABLE 2
Biological process enriched with genes repressed
by irradiation and reversed by simvastatin

| | Gene List 1 | | Gene List 2 | |
|---|---|---|---|---|
| GO ID - Function Name | # | q-value | # | q-value |
| GO: 0006511 ubiquitin-dependent | 22 | 1.6E−03 | 17 | 4.5E−02 |
| protein catabolic process | | | | |
| GO: 0006350 Transcription | 156 | 5.5E−04 | 125 | 8.2E−03 |
| GO: 0006397 mRNA processing | 41 | 3.1E−07 | 31 | 8.2E−03 |
| GO: 0007186 GPCR protein | 12 | 1.9E−03 | 8 | 8.4E−03 |
| signaling pathway | | | | |
| GO: 0006355 regulation of tran- | 144 | 8.4E−03 | 136 | 4.5E−02 |
| scription, DNA-dependent | | | | |
| GO: 0016568 chromatin modifica- | 25 | 1.9E−03 | 19 | 4.8E−02 |
| tion | | | | |
| GO: 0008380 RNA splicing | 31 | 3.1E−05 | 24 | 1.7E−02 |
| GO: 0008152 metabolic process | — | NS | 15 | 1.3E−02 |
| GO: 0007242 intracellular | — | NS | 36 | 1.7E−02 |
| signaling cascade | | | | |
| GO: 0006468 protein amino acid | — | NS | 31 | 1.4E−02 |
| phosphorylation | | | | |

Differentially expressed genes in Gene list 1 and 2 were identified by Significant Analysis of Microarray described in Table 1. The genes downregulated by irradiation in Gene list 1 or the genes upregulated by simvastatin in Gene list 2 were uploaded into Onto-Express software to identify overrepresented Gene Ontology (GO) categories. The significance is set at q-value < 0.05 with more than 6 genes in the biological process (see Methods).
* p-value adjusted by Benjamini-Hochberg approach to control multiple test.

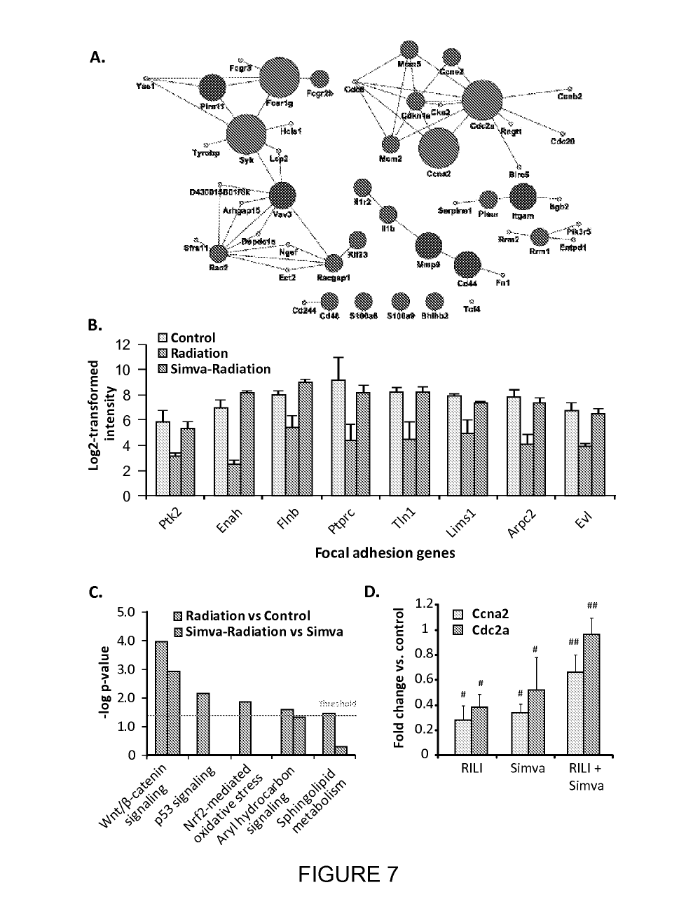

Support for the preclinical RILI model was obtained by filtering RILI- and simvastatin-influenced deregulated genes as an “interactome” of RILI-deregulated proteins, which identified four genes/proteins (CD44, Cdc2a, Syk, Ccna2) as key interactors that are significantly altered by exposure to ionizing radiation (FIG. 7A). Cdc2a and Ccna2 function as checkpoint genes critical to radiation effects in tissues; cytoplasmic spleen tyrosine kinase (Syk) functions as a tumor suppressor involved in responses to oxidative stress (Qin et al., 1998, Biochemistry 37:5481-5486) including endothelial cells (Foncea et al., 2000, Biological Research 33:89-96); and CD44 is a key regulatory receptor for hyaluronan involved in responses to lung injury including RILI (Iwakawa et al., 2004, J Radiat Res (Tokyo) 245:423-433; Sakai et al., 2008, Journal Radiat Res 49:409-416), and regulation of vascular permeability (Singleton et al., 2007, J. Biol. Chem. 282:30643-30657). A PubMed database blast (PubMatrix) was used to determine the number of citations involving prioritized RILI “interactome” gene/protein components confirmed that >50% of “interactome” components are associated with normal cellular responses to radiation (Table 3). The PubMatrix analysis verified the participation of these prioritized genes in cellular responses to radiation.

TABLE 3
PubMed database blast (PubMatrix) of potential gene/protein RILI interactome components

| Pub | | | | pulmonary | Radiation | Gene ID | |
|---|---|---|---|---|---|---|---|
| matrix | Radiation | irradiation | X-ray | fibrosis | pneumonitis | GenBank* | SEQ ID |
| CDC2a | 0 | 1 | 0 | 0 | 0 | 12534 | NOs: 1-4 |
| | | | | | | NM_007659 | |
| | | | | | | NM_001786^{†} | |
| ccna2 | 2 | 3 | 1 | 0 | 0 | 12428 | NOs: 5-8 |
| | | | | | | NM_009828 | |
| | | | | | | NM_001237^{†} | |
| syk | 22 | 23 | 19 | 1 | 0 | 20963 | NOs: 9-12 |
| | | | | | | NM_011518 | |
| | | | | | | NM_003177^{†} | |
| fcer1g | 1 | 1 | 0 | 0 | 0 | 14127 | NOs: 13-16 |
| | | | | | | NM_010185 | |
| | | | | | | NM_004106^{†} | |
| vav3 | 0 | 0 | 0 | 0 | 0 | 57257 | NOs: 17-20 |
| | | | | | | NM_020505 | |
| | | | | | | NM_006113^{†} | |
| CD44 | 145 | 194 | 28 | 22 | 2 | 12505 | NOs: 21-24 |
| | | | | | | NM_009851 | |
| | | | | | | NM_000610^{†} | |
| mmp9 | 12 | 13 | 11 | 6 | 0 | 17395 | NOs: 25-28 |
| | | | | | | NM_013599 | |
| | | | | | | NM_004994^{†} | |
| itgam | 99 | 110 | 17 | 11 | 0 | 16409 | NOs: 29-32 |
| | | | | | | NM_001082 | |
| | | | | | | 960 | |
| | | | | | | NM_001145 | |
| | | | | | | 808^{†} | |

PubMatrix analysis of selected prioritized interactome genes/proteins depicted in FIG. 7A. These interacting proteins were blasted against PubMatrix headers reflecting radiation responses. The majority of these genes reflect involvement in normal cellular responses to radiation.
*The GenBank accession numbers provided are exemplary.
^{†}Human sequences.

Gene Ontology enrichment analysis revealed simvastatin normalization of radiation-induced down-regulation of the focal adhesion pathway (7 genes), highly relevant to regulation of lung barrier integrity (FIG. 7B), and Ingenuity pathway analysis of up-regulated RILI genes revealed robust activation of 5 canonical pathways (wnt/β-catenin, p53, aryl hydrocarbon receptor, Nrf2 signaling, sphingolipid metabolism) with each deregulated pathway either attenuated or completely reversed by simvastatin (FIG. 7C). RILI-associated deregulation of the nuclear factor-erythroid-2-related factor 2 (Nrf2) pathway was consistent with the increased ROS/RNS observed in the preclinical model. Finally, radiation-mediated transcriptional inhibition of the cell cycle genes, Cdc2 and Ccna2, was validated by RT-PCR (FIG. 7D) with the inhibition reversed by simvastatin. Ccna2 and Cdc2 were identified by “Single Network Analysis of Proteins” (SNAP), a protein-protein interaction-network analysis used to identify most deregulated protein network in radiated and simvastatin-treated lungs using the signature genes, network topology, and expression dynamics family. Quantification was performed by TaqMan real-time RT-PCR assays and 7900HT Fast Real-time PCR system (Applied Biosystems, Foster City, Calif.).

Overall, simvastatin potently suppressed radiation-induced gene stress pathways (Wnt-β catenin-, Nrf2-, p53-signaling pathways) via transcriptional reprogramming of radiation-dysregulated genes, findings compatible with reports of simvastatin-mediated down-regulation of chemokine and chemokine receptor expression (Jacobson et al., 2004, Am J Respir Cell Mol Biol 30:662-670) whose expression on the endothelial surface is increased by radiation (Kureishi et al., 2000, Nat Med 6:1004-1010). Quantitative and qualitative changes in RILI gene expression resulted in cytokine overproduction, which in autocrine and paracrine fashion increase mRNA translation (Lu et al., 2006, Cancer Research 66:1052-1061) triggering a cascade leading to RILI pathobiology. Simvastatin normalized radiation-induced Nrf2 deregulation, which is essential for the coordinated induction of genes encoding stress-responsive and cytoprotective proteins (Dinkova-Kostova et al., 2008, Mol Nutr Food Res 52 Suppl 1:S128-138; Cho et al., 2006, Antioxid Redox Signal 8:76-87).

### Example 5

### Identification of sphingosine-1-phosphate (S1P) pathway-related biomarkers in RILI

Potential S1P pathway-related RILI biomarkers were investigated. Lung tissues were homogenized in a polytron in a buffer containing: 20 mM Tris-HCl (pH 7.4), 150 mM NaCl, 2 mM EGTA, 5 mM (3-glycerophosphate, 1 mM MgCl₂, 1% Triton X-100, 1 mM sodium orthovanadate, 10 μg/ml protease inhibitors, 1 μg/ml aprotinin, 10 μg/ml leupeptin, and 1 μg/ml pepstatin. Lysates were centrifuged at 500×g for 5 min at 4° C., and equal amounts of protein (20 μg) were loaded onto 10% SDS/PAGE gels and Western blotting performed according to standard protocols.

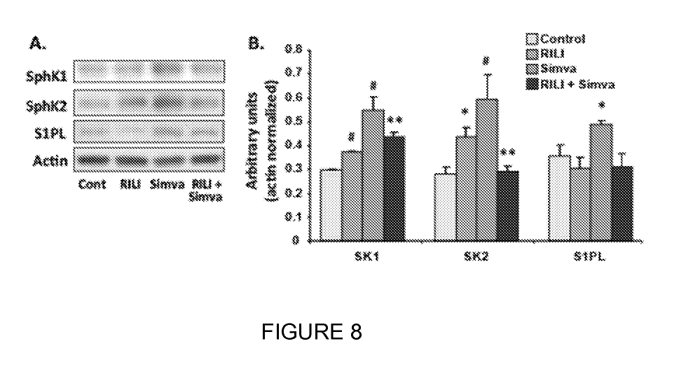

Simvastatin treatment (10 mg/kg IP, 3×/wk beginning 1 week prior to irradiation) resulted in a significant increase in lung SphK1 but a significant decrease in lung SphK2 at 6 weeks compared to RILI-challenged controls (FIGS. 8A and 8B). These data suggest that SphK1 and SphK2 are useful clinical biomarkers.

Unexpectedly, SphK1 expression was further augmented in RILI-challenged mice that received simvastatin as compared with RILI mice without treatment while expression of SphK2 was significantly reduced by simvastatin. Given the primacy for SphK1 in increasing cellular S1P levels compared to SphK2 (which has been inferred to participate in apoptosis; Zhao et al., 2007, J Biol. Chem. 282:14165-77), these findings suggested that simvastatin reversed the RILI-mediated dysregulation of the sphingolipid rheostat.

### Example 6

### Analysis of S1P, DHS1P, and Ceramide in Biological Fluids

The effects of radiation on ceramide and S1P levels in biologic fluids were analyzed via combined LC/MS/MS on an API4000 Q-trap hybrid triple quadrupole linear ion-trap mass spectrometer (Applied Biosystems, Foster City, Calif.) equipped with a turbo ion spray ionization source interfaced with an automated Agilent 1100 series liquid chromatograph and autosampler (Agilent Technologies, Wilmington, Del.). The sphingolipids were ionized via electrospray ionization (ESI) with detection via multiple reactions monitoring (MRM). Analysis of sphingoid bases employed ESI in positive ions with MRM analysis and was conducted as follows (see also Berdyshev et al., 2005, Anal Biochem. 339:129-36; Berdyshev et al., 2006, Cell Signal. 18:1779-92). Positive ion ESI LC-MS/MS analysis was employed for detection of S1P as the sphingoid base-1-phosphate. The ion source conditions and gas settings for positive ESI LC-MS/MS analysis were as follows: ion spray voltage=5500 V, ion source heater temperature=520° C., collision gas setting=4, ion source gases 1 and 2 settings=50, curtain gas setting=10. The MRM transition monitored for detection of S1P was m/z 380/264. Optimized parameters for S1P positive ion ESI LC-MS/MS analysis were as follows: declustering potential=46 V, collision energy=21 V, collision exit potential=26 V. Several different C18 and C8 reversed-phase columns of various lengths, inside diameters, and particle sizes of packing material were employed in initial studies in attempting to eliminate carryover from a previous injection or previous injections. In addition, several solvent gradient systems were evaluated for possible elimination of previous sample carryover.

Negative ion ESI LC-MS/MS analysis was employed for detection of bisacetylated sphingoid base-1-phosphates. The ion source and gas settings for negative ion ESI LC-MS/MS analysis were as follows: ion spray voltage=−4500 V, ion source heater temperature=520° C., collision gas setting=4, ion source gases 1 and 2 settings=50, curtain gas setting=10. The MRM transitions monitored were as follows: C17-S1P (internal standard) m/z 448/388, S1Pm/z 462/402, DHS1P m/z 464/404. The optimal declustering potentials for C17-S1P, S1P, and DHS1P were −135, −140, and −140 V, respectively. The optimal collision energies for C17-S1P, S1P, and DHS1P were −26, −28, and −28 V, respectively. The optimal collision exit potentials were −9 V for C17S1P and −11 V for both S1P and DHS1P. Liquid chromatographic resolution of C17S1P, SIP, and DHS1P as bisacetylated derivatives, either as a mixture of standards or extracted from biological matrices, was achieved via the use of an Agilent Zorbax Eclipse XDB-C8 column (150×4.6 mm, 5 μm particle size) employing gradient elution. A mixture of water/methanol/formic acid (20:80:0.5, v/v) containing 5 mM ammonium formate was used as solvent A, and methanol/acetonitrile/formic acid (59:40:0.5, v/v) containing 5 mM ammonium formate was used as solvent B. The elution protocol was composed of a 2-min column equilibration with 100% solvent A, followed by sample injection in methanol, a 2-min period with 100% solvent A, a 3-min linear gradient to 100% solvent B, a 3-min period with 100% solvent B, and a 2-min linear gradient to 100% solvent A. The solvent flow rate was 0.5 ml/min. The program included three cyclic needle washes consisting of duplicate needle washes per cycle prior to sample injection.

Standard curves of S1P and DHS1P, with C17-S1P as the internal standard, were constructed by adding increasing concentrations of S1P and DHS1P to 40 pmol of the internal standard, followed by treatment with acetic anhydride. Two sets of standard curves were obtained. One set was obtained in the absence of a biological matrix, and the second set was obtained in the presence of total lipids extracted from human pulmonary artery endothelial cells (HPAECs) (4 nmol total lipid phosphorus per vial). Linearity of the standard curves and correlation coefficients were obtained by linear regression analysis.

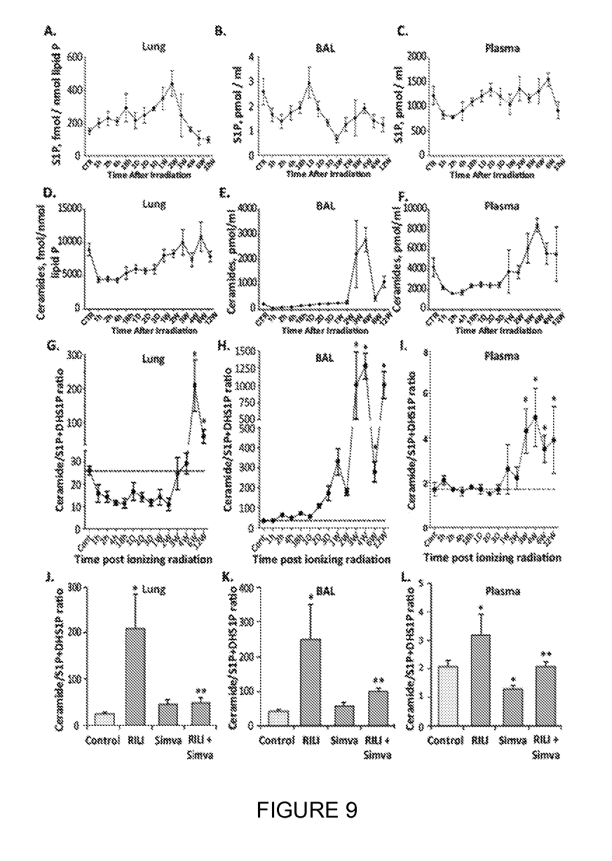

S1P levels were significantly increased at 1 week post-radiation in lung homogenates and decreased significantly in BAL at this same time point (FIGS. 9A-9C). There were no significant changes detected in plasma at any time point (FIG. 9B). In contrast, ceramide levels were significantly decreased<1 wk post-radiation in lung homogenates but were unchanged at later time points (FIG. 9D) while BAL ceramide levels were significantly increased at 3-4 weeks (FIG. 9E). Again, however, there were no significant changes detected in plasma at any time point (FIG. 9F). Nonetheless, the ratio of ceramide to cumulative S1P and DHS1P levels was significantly increased 3-6 weeks post radiation in lung homogenates and BAL fluid and plasma (FIG. 9G-I). The increases in ceramide/(S1P+DHS1P) ratio was attenuated in mice that received simvastatin (10 mg/kg body weight, 3×/wk) or vehicle beginning 1 week prior to radiation (25 Gy, single thoracic dose) and continued up to 6 weeks post-irradiation. Overall, simvastatin treatment significantly altered ceramide/S1P-DHS1P ratio in lung tissue, BAL fluids and plasma of animals exposed to radiation as compared to animals receiving radiation alone. These results provide further evidence that the beneficial effects of simvastatin in RILI may be linked to an attenuation of radiation-mediated changes in sphingolipid metabolism.

Previously, RILI has been hypothesized to be the result of a sustained cytokine cascade due to an inflammatory response activated by radiation, with a large body of experimental data implicating a number of chemokines and cytokines including TGFβ, IL-1α, IL-6, and TNFα (Anscher et al., 1998, Int J Radiat Oncol Biol Phys. 41:1029-35; Chen et al., 2002, Semin Radiat Oncol. 12:26-33; Rubin et al., 1995, Int J Radiat Oncol Biol Phys. 33:99-109). However, neither the prediction nor the amelioration of radiation pneumonitis has been consistently correlated with cytokine levels or specific neutralization of individual cytokines (Ogata et al., 2010, Radiat Oncol. 5:26). These findings suggest that sphingolipids may serve as clinical biomarkers for both RILI and to monitor responses to therapy.

### Example 7

### Role of Sphingolipid Pathway Components in RILI Pathogenesis

To further characterize the role of specific sphingolipid pathway components in the elaboration of RILI, genetically-engineered mice with complete or partial targeted deletion of alleles for SphK1 (SphK1^{-/-}), S1PR1 (S1PR^{+/-}), S1PR² (S1PR^{2-/-}), or S1PR³ (S1PR^{3-/-}) were exposed to a single dose of thoracic irradiation (10-25 Gy), then their responses assessed at 4-6 weeks. Mice were anesthesized with ketamine (100 mg/kg) and acepromazine (1.5 mg/kg) and administered radiation (10-25 Gy) to the thorax (Mathew et al., 2010, Am J Respir Cell Mol. Biol. 2010 May 27, ePublicatino only, PMID: 20508068). A 5 mm thick lead block was used to shield the rest of the animal while the thorax, between the clavicles and below sternum, was irradiated with 250 kV x-ray beam at a dose rate of 2 Gy/min using an orthovoltage animal irradiator. Each experimental group consisted of 10 mice irradiated to a single dose of 10, 20, or 25 Gy. The variation of the dose delivered within the lung was estimated to be within ±5% of the prescribed dose using thermoluminescence dosimeters. Mice were sacrificed and indices of lung vascular leak and inflammation assessed via BAL fluid protein levels and cell counts at 4-6 weeks as described above (see also Nonas et al., 2007, Am J Physiol Lung Cell Mol. Physiol. 293:L292-302). Lungs were harvested and stored at −80° C. for histologic evaluation.

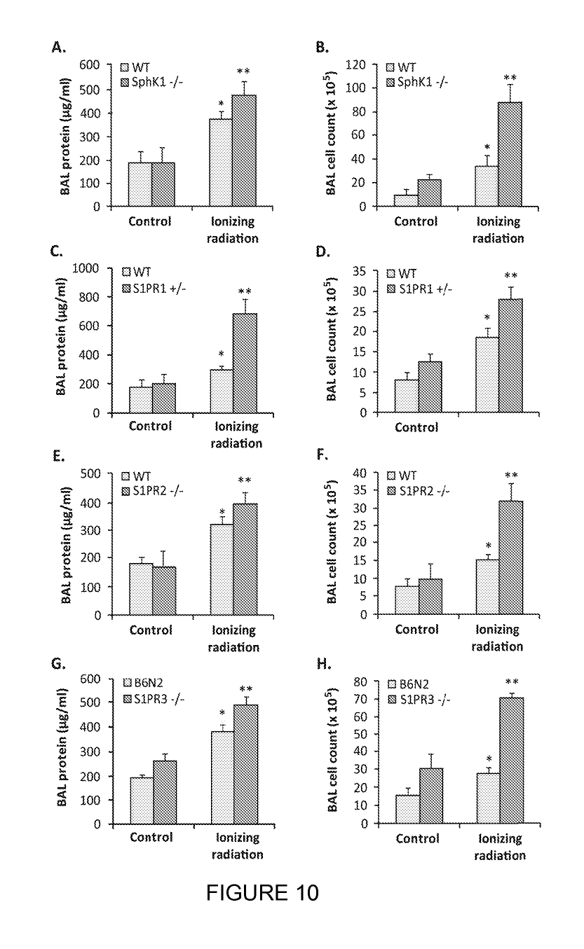

While the differences in BAL protein and cell counts were not significant at baseline, these indices were significantly increased at 4-6 weeks post-radiation in each group compared to wild type controls consistent with increased susceptibility to RILI in S1P pathway-modified mice (FIG. 10). The degree of increased RILI susceptibility was relatively comparable across the strains of genetically-engineered mice albeit at variable time points and radiation dosing.

The observation that genetically-engineered mice with targeted S1P receptor deletions, including S1PR1, S1PR2, S1PR3, as well as mice with targeted SphK1 deletions, all demonstrated increased susceptibility to RILI is consistent with the importance of the sphingolipid pathway in RILI pathogenesis. The observed deleterious effects of S1PR2 or S1PR3 depletion contrast with results of prior studies showing that S1PR2^{-/-} and S1PR3^{-/-} mice exhibit reduced injury in a LPS-induced preclinical acute lung injury (ALI) model (Sammani et al., 2010, Am JRespir Cell Mol Biol 43(4):394-402). In those studies, S1PR2^{-/-} mice administered intratracheal LPS were found to have elevated BAL fluid total protein levels compared to LPS-treated wildtype animals although no difference was detected with respect to BAL cell counts. Similarly, in animals administered S1PR3 siRNA via ACE antibody-conjugated nanocarriers, LPS-induced elevations in BAL fluid albumin and total protein levels were significantly reduced compared to LPS-treated controls (Sammani et al., 2010, Am J Respir Cell Mol Biol 43(4):394-402). These results strongly suggest unique roles for S1PR2 and S1PR3 in specific models of murine inflammatory lung injury.

A potential explanation for the conflicting roles of S1P receptors in ALI and RILI responses is that alveolar epithelial cell barrier function and vascular endothelial cell barrier function are distinct in susceptibility to ionizing radiation and LPS treatment. It is speculated that S1PR2 and S1PR3 likely exert complex, possibly injury-, cell- and species-specific barrier-regulatory properties, potentially due to their ability to activate multiple multimeric G proteins (Sanchez and H1a, 2004, J Cell Biochem. 92:913-22). Nonetheless, these data suggested that S1P receptors were critical to RILI pathobiology, and that S1P-related compounds could be effective and novel therapeutic agents for radiation pneumonitis.

### Example 8

### Effect of S1P Analogs on Severity of Murine RILI

Despite its known vascular-protective effects both in vivo and in vitro, endogenous S1P produces a myriad of effects, including several that limit its usefulness as a therapeutic agent in patients. Given these limitations for S1P, structurally similar compounds such as FTY720 (FTY), SEW2871 (an S1PR1 agonist), and (S)-FTY720-phosphonate were evaluated as potential novel therapies for RILI.

The vascular-protective effects of FTY720 were previously reported in a murine model of lipopolysaccharide (LPS)-induced acute lung injury (<24 hrs) (Peng et al., 2004, Am J Respir Crit. Care Med. 169:1245-51). Subsequent studies in chronic models (2 weeks) of lung injury have suggested that FTY720 induces increased lymphopenia and bradycardia, as well as mortality, thereby limiting its potential utility in human disease (Kovarik et al., 2004, Ther Drug Monit. 26:585-7; Matloubian et al., 2004, Nature. 427:355-60). Other S1P receptor agonist SEW 2871 and S1P analog (S)-FTY720-phosphonate were also shown to reduce vascular leak and inflammation both in vitro and in vivo in murine models of LPS-induced ALI as well as in a pre-clinical brain death model of lung injury (Camp et al., 2009, J Pharmacol Exp Ther. 331:54-64; Sammani et al., 2010, Am J Respir Cell Mol Biol 243 (4):394-402).

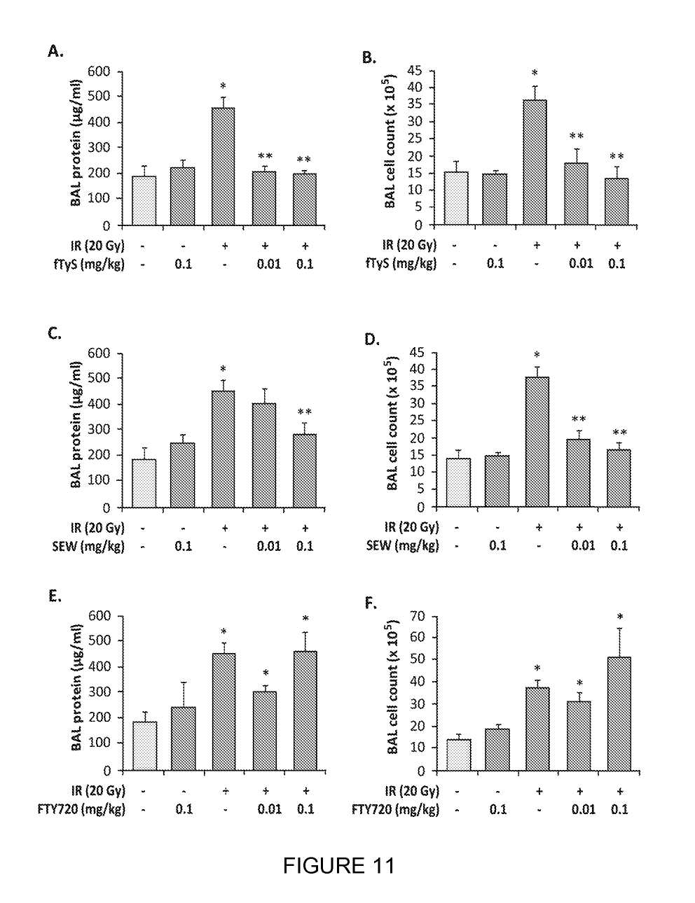

To investigate the effects of FTY720, SEW 2871 and (S)-FTY720-phosphonate in murine RILI, these compounds were administered in either low or high concentrations (0.01 or 0.1 mg/kg, respectively) to RILI-challenged mice and assessed at 6 weeks via BAL fluid protein levels and cell counts (FIG. 11). Mice were anesthesized with ketamine (100 mg/kg) and acepromazine (1.5 mg/kg) and administered radiation (10-25 Gy) to the thorax. A 5 mm thick lead block was used to shield the rest of the animal while the thorax, between the clavicles and below the sternum, was irradiated with 250 kV x-ray beam at a dose rate of 2 Gy/min using an orthovoltage animal irradiator. Each experimental group consisted of 10 mice irradiated to a single dose of 10, 20, or 25 Gy. The variation of the dose delivered within the lung was estimated to be within ±5% of the prescribed dose using thermoluminescence dosimeters. Select mice were treated with 0.01 or 0.1 mg/kg FTY720, SEW 2781, or (S)-FTY720-phosphonate via intraperitoneal injection 2×/week beginning 1 week prior to irradiation and continuing for a period up to 6 weeks afterwards. Mice were then sacrificed and indices of lung vascular leak and inflammation assessed via BAL fluid protein levels and cell counts at 4-6 weeks as described above. Lungs were harvested and stored at −80° C. for histologic evaluation. To characterize histological alterations, lungs from each experimental group were inflated to 30 cm H₂O with 10% formalin for histological evaluation by hematoxylin and eosin staining.

The results shown in FIG. 11 confirmed the dose-dependent protective effects of both (S)-FTY720-phosphonate and SEW2871. Compared to RILI-challenged controls, mice treated with (S)-FTY720-phosphonate had significant decreases in both BAL protein and cell counts at both high and low dosing while treatment with SEW2871 resulted in a dose-dependent protective effect. Surprisingly, in contrast, FTY720 did not confer significant protection at comparable concentrations.

To assess microvascular changes associated with RILI in real-time, mice were imaged with ViSen FMT 1500 Quantitative Tomography In Vivo Imaging System. Intergisens⁷⁵⁰ NIR (ViSen Medicals, Bedford, Mass.) was used as a probe, which targets the vasculature as a selective non-peptide small molecule integrin αᵥβ₃ antagonist and a near-infrared fluorochrome. Mice were injected with probe (2 nM, IV) at 6 weeks post radiation and imaged 24 hrs later. Probe intensity was quantified using TrueQuant 3D software (VisSen Medicals).

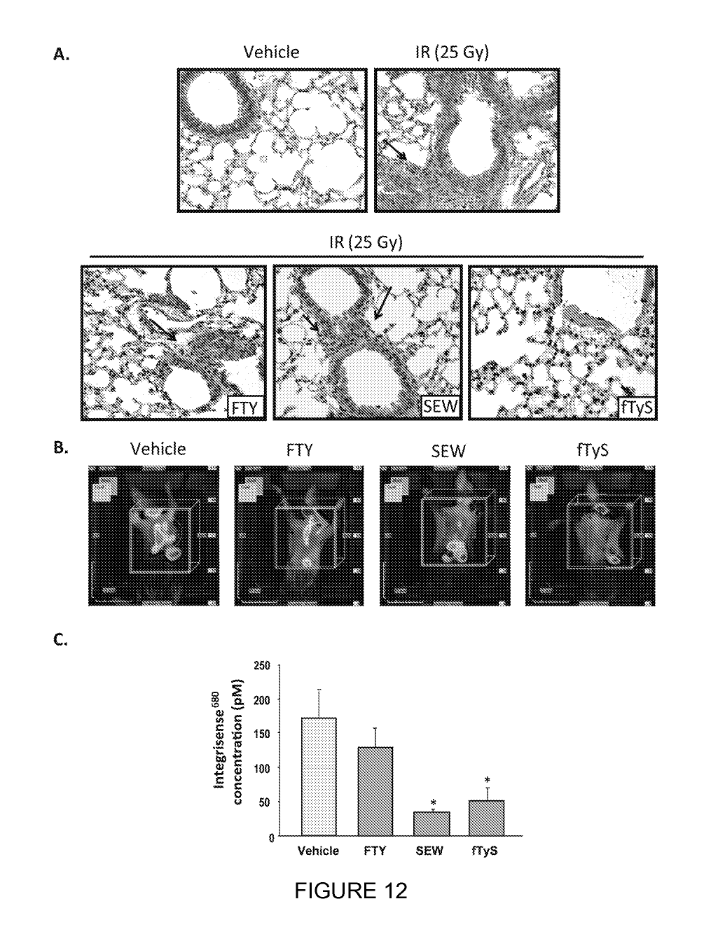

Lung histological examination as well as ViSen FMT lung imaging corroborated biochemical and cellular levels of S1P analog protection at 6 weeks (FIG. 12) with abundant areas of inflammatory cell infiltration into the lung interstitium and modest interstitial edema induced by radiation, which were markedly attenuated by (S)-FTY720-phosphonate (0.1 mg/kg, i.p., administered 2×/wk beginning one week prior to irradiation) and to a lesser extent by SEW 2871 and FTY720. Separately, ViSen FMT imaging demonstrated significant probe signal localized to the thorax in RILI-challenged mice consistent with increased lung vascular permeability. Quantification of probe intensity confirmed significant decreases in radiation-induced probe extravasation in animals treated with either SEW 2871 or (S)-FTY720-phosphonate (0.1 mg/kg), consistent with the vascular barrier-protective effects of these compounds. In contrast, there was no evidence of protection in animals treated with FTY720 (0.1 mg/kg) compared to RILI controls. Thus, surprisingly, FTY720, which has been shown to protect against LPS-induced acute lung injury (see), failed to confer comparable protection against RILI.

### Example 9

### Modulation of RILI-Induced Lung Gene Dysregulation by S1P Analogs

To link the protective effects of the S1P analogs in murine RILI to genomic influences of these interventions, genome-wide expression profiling of lung tissues after RILI were conducted. Total RNA was isolated from whole lungs for expression profiling (Nonas et al., 2007, Am J Physiol Lung Cell Mol. Physiol. 293:L292-302) using Affymetrix Mouse 430 2.0 arrays and protocols (Affymetrix, Santa Clara, Calif., USA). Chips were scanned using a GeneChip Scanner 3000 (Affymetrix). Chip quality and “present” calls were determined by Affymetrix GCOS software. The chip data were normalized by “rank invariant set” method using dChip software (Li and Hung Wong, 2001, Genome Biol. 2:RESEARCH0032). The potential batch effect was corrected by Combat software (Johnson et al., 2007, Biostatistics. 8:118-27). The microarray data have been submitted to the Gene Expression Omnibus (GEO) repository of the National Center for Biotechnology Information and have been published (GSE25295). The differentially expressed genes between two experimental groups were identified using Significance Analysis of Microarrays (SAM) (Tusher et al., 2001, Proc Natl Acad Sci USA. 98:5116-21).

Dysregulated genes were uploaded into the Ingenuity Pathway Analysis (IPA) software, a web-delivered application that utilizes the Ingenuity Pathways Knowledge Base (IPKB) containing a large amount of individually modeled relationships between gene objects, e.g., genes, mRNAs, and proteins, to dynamically generate significant regulatory and signaling networks or pathways (Meyer et al., 2009, Faseb J 23:1325-1337). The genes submitted for mapping to corresponding gene objects in the IPKB are called “focus genes.” The significance of a canonical pathway is controlled by P value, which is calculated using the right-tailed (referring to the overrepresented pathway) Fisher's exact test for 2×2 contingency tables. This is done by comparing the number of focus genes that participate in a given pathway, relative to the total number of occurrences of those genes in all pathways stored in the IPKB. The significance threshold of a canonical pathway is set to 1.3, which is derived by −log₁₀ [P value], with P≦0.05. Principle component analysis (PCA) on the experimental conditions was performed using R package “Ade4”.

Differentially expressed genes were identified by two-group comparison using SAM software and 92 up- and 158 down-regulated genes were identified in response to radiation alone at 6 weeks (≧1.8 fold change, 5% FDR) (Table 4). Differentially expressed genes were identified by two-group comparison using SAM software. The results are shown and full gene lists are provided online: phenos.bsd.uchicago.edu/publication/Radiation_{—}3DrugComparison/(1R=irradiation, CTR=control, FDR=false discovery rate, FC=fold change).

TABLE 4
Genomic Changes Associated with RILI and
Treatment with Sphingolipid Analogs

| Comparison | FDR | FC | Probe sets | UP | DN |
|---|---|---|---|---|---|
| IR vs CTR | 5% | 1.8 | 250 | 92 | 158 |
| IR-FTY vs CTR | 5% | 1.8 | 420 | 186 | 234 |
| IR-SEW vs CTR | 5% | 1.8 | 10 | 7 | 3 |
| IR-fTyS vs CTR | 5% | 1.8 | 3 | 3 | 0 |
| FTY vs CTR | 5% | 1.8 | 1 | 0 | 1 |
| SEW vs CTR | 5% | 1.8 | 44 | 8 | 36 |
| fTyS vs CTR | 5% | 1.8 | 19 | 19 | 0 |
| IR-fTyS vs IR | 7% | 1.8 | 103 | 50 | 53 |

The 250 RILI dysregulated genes were uploaded into Ingenuity software and deregulated canonical pathways identified (Tables 5 and 6) including leukocyte extravasation signaling, IL-10 signaling, and HIF1α signaling. The most highly down-regulated pathways included B cell development and protein kinase A (PKA) signaling. The 92 probe sets up-regulated in response to radiation compared to control were uploaded into Ingenuity software to identify deregulated canonical pathways. The most prominently represented pathways are shown (Table 5).

The 158 probe sets down-regulated in response to radiation compared to control were uploaded into Ingenuity software to identify deregulated canonical pathways. The most prominently represented pathways are shown (Table 6).

TABLE 5
Identification of deregulated canonical pathways

| | −log | |
|---|---|---|
| Canonical Pathways | (p-value) | Molecules |
| Leukocyte Extravasation | 3.70 | ITGAM, NCF2, NCF4, |
| Signaling | | MMP12, MMP9, SELPLG |
| Atherosclerosis Signaling | 2.88 | IL1B, CCR2, MMP9, |
| | | SELPLG |
| TREM1 Signaling | 2.63 | TREM1, TYROBP, IL1B |
| IL-10 Signaling | 2.49 | CCR1, FCGR2A, IL1B |
| Dendritic Cell Maturation | 2.38 | FCGR2A, TYROBP, |
| | | FCER1G, IL1B |
| LXR/RXR Activation | 2.36 | IL1B, APOC2, MMP9 |
| Natural Killer Cell Signaling | 2.05 | TYROBP, CD244, |
| | | FCER1G |
| Graft-versus-Host Disease | 1.97 | FCER1G, IL1B |
| Signaling | | |
| HIF1α± Signaling | 1.94 | MMP12, MMP9, SLC2A3 |
| Systemic Lupus Erythematosus | 1.83 | FCGR2A, FCER1G, IL1B |
| Signaling | | |
| Airway Pathology in COPD | 1.45 | MMP9 |
| Communication between Innate | 1.45 | FCER1G, IL1B |
| and Adaptive Immune Cells | | |
| Eicosanoid Signaling | 1.40 | FPR2, ALOX5AP |
| Role of Pattern Recognition | 1.36 | NLRP3, IL1B |
| Receptors in Recognition of | | |
| Bacteria and Viruses | | |
| Pathogenesis of Multiple | 1.35 | CCR1 |
| Sclerosis | | |
| IL-8 Signaling | 1.34 | ITGAM, NCF2, MMP9 |

The upregulation of HIF1α signaling by radiation in the RILI mouse model was notable as HIF1α has previously been found to be upregulated in the lungs of rats administered thoracic radiation and was also found to correlate with the degree of lung inflammation in these animals (Rabbani et al., 2010, Radiat Res. 173:165-74). In addition, the downregulation of protein kinase A (PKA) signaling by radiation is consistent with the important role for this pathway in responses to radiation as evidence of increased PKA expression has been linked to poor clinical responses to radiotherapy in some patient populations (Pollack et al., 2009, Clin Cancer Res. 15:5478-84).

TABLE 6
Identification of deregulated canonical pathways

| | −log | |
|---|---|---|
| Canonical Pathways | (p-value) | Molecules |
| Primary Immunodeficiency | 3.79 | LCK, IGKC, IGHM, |
| Signaling | | IGK-V28 |
| B Cell Development | 3.06 | IGKC, IGHM, IGK-V28 |
| Glycerophospholipid | 2.66 | PLCB4, DGKD, |
| Metabolism | | PLA2G2D, CHKA, |
| | | ETNK1 |
| Systemic Lupus | 2.20 | LCK, IGKC, IGHM, |
| Erythematosus Signaling | | IGK-V28 |
| Melatonin Signaling | 1.95 | PRKACB, PLCB4, RORA |
| Cellular Effects of Sildenafil | 1.93 | SLC4A5, PRKACB, PLCB4, |
| | | MYH2 |
| Protein Kinase A Signaling | 1.83 | PRKACB, PLCB4, MYH2, |
| | | PDE7A, CREBBP, AKAP9 |
| Citrate Cycle | 1.73 | SUCLA2, PCK1 |
| G Protein Signaling Mediated | 1.65 | LCK, PLCB4 |
| by Tubby | | |
| Phospholipid Degradation | 1.65 | PLCB4, DGKD (includes |
| | | EG: 8527), PLA2G2D |
| D-glutamine and D-glutamate | 1.54 | GLS |
| Metabolism | | |
| Mechanisms of Viral Exit | 1.53 | CHMP4C, NEDD4 |
| from Host Cells | | |
| Synaptic Long Term | 1.43 | PRKACB, PLCB4, CREBBP |
| Potentiation | | |
| Estrogen Receptor Signaling | 1.40 | CREBBP, PCK1, NR3C1 |

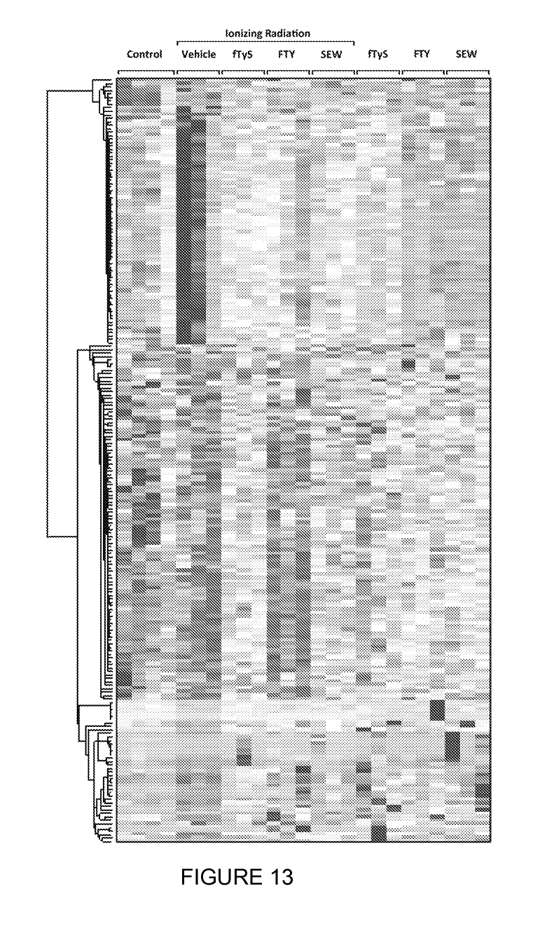

Heat map analysis of S1P analog effects on RILI gene expression (FIG. 13) revealed significant radiation-mediated genomic effects that were strongly attenuated with varying potency by S1PR1 agonism via FTY720, SEW 2871, and (S)-FTY720-phosphonate (0.1 mg/kg). Consistent with the corresponding physiologic data, treatment with (S)-FTY720-phosphonate and SEW 2871 significantly blunted the effects of radiation on lung gene dysregulation while FTY720 had only a marginal effect. Whereas analog effects were minimal in the absence of radiation, the effects of (S)-FTY720-phosphonate on radiation-induced gene expression changes were the most robust, with 54 genes significantly dysregulated by both radiation alone (compared to controls) and by (S)-FTY720-phosphonate in irradiated animals compared to radiation alone. Remarkably, all of these genes demonstrated opposing directional changes in these two analyses as there were 33 genes up-regulated in response to radiation that were down-regulated in response to (S)-FTY720-phosphonate and 21 genes down-regulated in response to radiation that were up-regulated by (S)-FTY720-phosphonate. Included in these gene sets were IL-1β, a gene previously found to be markedly upregulated in the lungs of mice subjected to a single dose of thoracic irradiation (Hong et al., 1999, Int J Radiat Biol. 75:1421-7), and MMP-9, a gene previously identified as activated in the murine RILI model (Mathew et al., 2010, Am J Respir Cell Mol. Biol. ePublication only, PMID: 20508068). IL-1β and MMP-9 were both upregulated by radiation compared to controls (4.4 and 3.8 fold change, respectively) but down-regulated by (S)-FTY720-phosphonate in irradiated mice compared to untreated irradiated control animals (0.33 fold change for both).

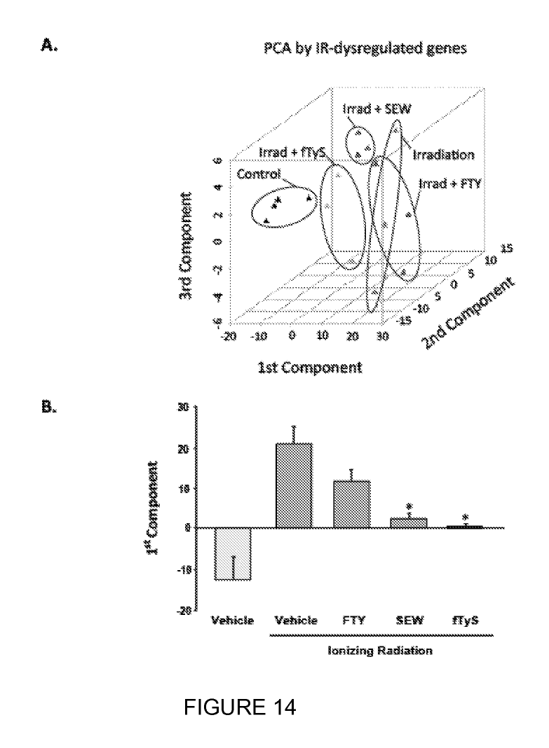

To further characterize the effects of the S1P analogs on genomic changes induced by radiation, principal component analysis (PCA) was performed using the 250 probe sets dysregulated by radiation exposure (FIG. 14). In a 3D scatter plot of the PCA analysis, the first component represents the primary variable affecting sample conditions (lung injury induced by radiation). Two-group comparison by t-test between radiation alone and uninjured controls as well as between each drug-treated, irradiated group and radiation alone revealed the principle component of the radiation alone (25 Gy) group was substantially higher than uninjured controls (as expected), but was significantly reduced at 6 weeks by both SEW 2871 and (S)-FTY720-phosphonate interventions (0.1 mg/kg) but not by FTY720 (p=0.07). Moreover, these data suggest a more potent effect of (S)-FTY720-phosphonate on RILI compared to SEW 2871, as the (S)-FTY720-phosphonate—treated samples are grouped more closely to the controls with respect to the first component. There were no significant differences between the principle component of radiation with FTY720 and radiation alone, indicating that, similar to its effects on direct histologic and biochemical indices of lung injury, the genomic effects of FTY720 on the radiation response were marginal.

### Example 10

### Comparative Studies of Protective Effects of FTY720 Analogues and Simvastatin in Murine RILI

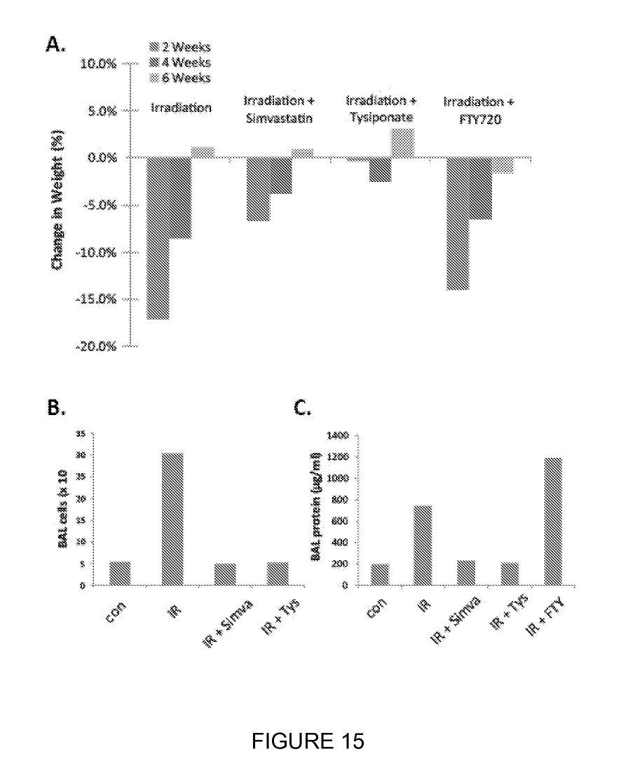

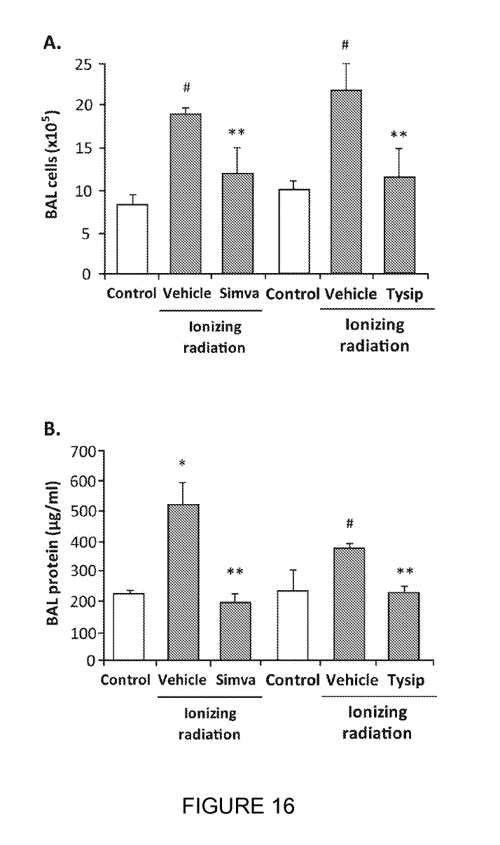

To compare the effects of FTY720 analogues and simvastatin on murine RILI, C57B1/6 mice were pretreated with (S)-FTY720-phosphonate (fTyS), FTY720 (0.1 mg/kg, i.p.) and simvastatin (10 mg/kg), 3 times/wk beginning one week before irradiation (20 i.p.) and continued up to 6 wks post-radiation. Irradiation resulted in significantly increased body weight loss in mice compared to mice treated with simvastatin or (S)-FTY720-phosphonate after irradiation (n=5, p<0.01), while FTY720 treatment did not show any beneficial effect in RILI as evidenced by significantly decreased weight loss compared to RILI-challenged controls and simvastatin- and (S)-FTY720-phosphonate-treated mice (FIG. 15A). Interestingly, the (S)-FTY720-phosphonate-treated mice looked overall healthier and did not lose weight. Overall, (S)-FTY720-phosphonate (compared to simvastatin) had a beneficial effect to mice undergoing radiation (weight and overall health). Similarly, mice treated with (S)-FTY720-phosphonate or simvastatin had significant decreases in both BAL cell counts and protein levels (FIG. 16B). In contrast, treatment with FTY720 did not confer significant protection (n=5/group, * p<0.05 compared to RILI controls).

### Example 11

### Direct Comparison of the Effects of Simvastatin and (S)-FTY720-Phosphonate on Lung Vascular Leakage and Inflammation in RILI Mice

To directly compare the protective effects of (S)-FTY720-phosphonate and simvastatin in murine RIL1, C57B1/6 mice were pretreated with (S)-FTY720-phosphonate (fTyS) and simvastatin (10 mg/kg) 3 times/wk beginning one week before irradiation (20 Gy) and continued up to 6 wks post-irradiation. Mice treated with (S)-FTY720-phosphonate and simvastatin had significant decreases in both BAL cell count and protein (FIG. 16).

### Example 12

### Effects of S1P Analogs on S1P Receptor 1 (S1PR1) Levels

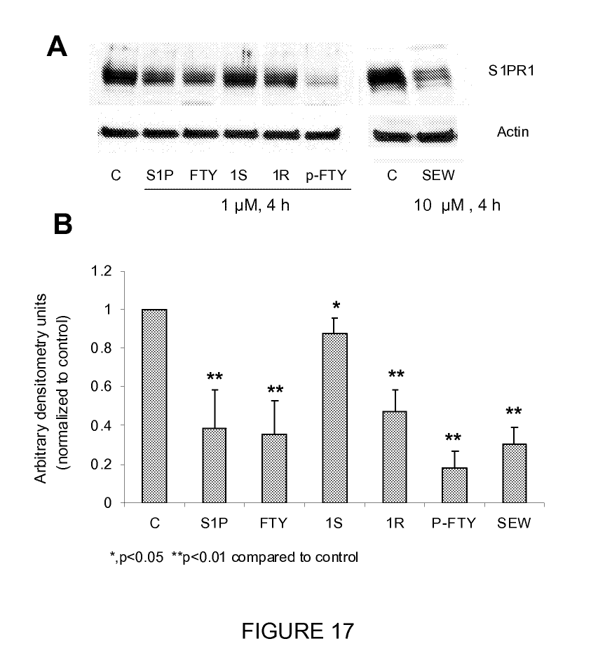

To assess the effects of the various S1P receptor 1 (S1PR1) agonists on S1PR1 protein expression, HPAECs were treated with 1 μM S1P, FTY720 (FTY), (S)-FTY720-phosphonate (1S), 1R, p-FTY720 (p-FTY), or 10 μM SEW2871 (SEW) for 4 h. Cells were collected and S1PR1 expression level was detected by Western blot. As shown in FIG. 17A, the expression of S1PR1 in the cells treated with S1P, FTY720, 1R, p-FTY or SEW2871 was significantly down-regulated. The expression level compared to control was 38.1% for S1P, 35.0% for FTY720, 47.2 for 1R, 17.7 for p-FTY or 30.3% for SEW2871 respectively. However, in sharp contrast, 1S treated cells still maintained S1PR1 expression (87.6% compared to control).

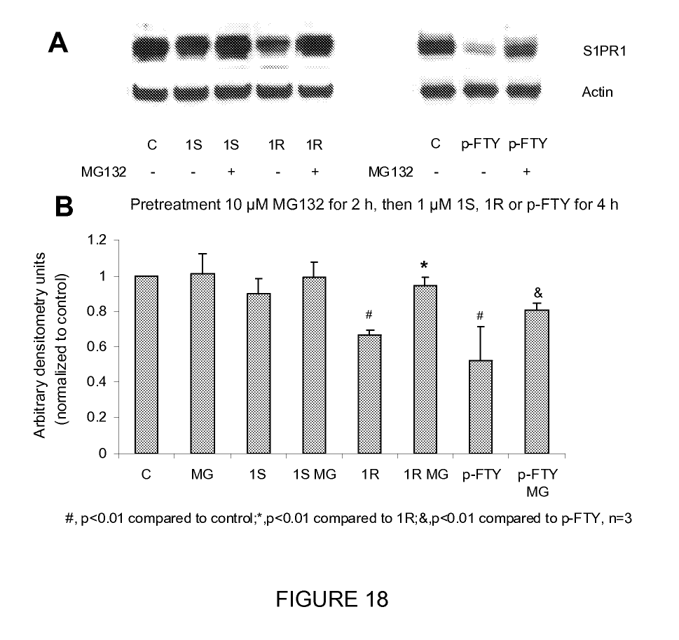

To assess the effects of the proteasome specific inhibitor MG132 administered in conjunction with S1PR1 agonists on the expression of S1PR1, HPAECs were pretreated with MG132 (10 μM) for 2 h and then treated with 1 μM S1P, FTY720, (S)-FTY720-phosphonate (1S), 1R, p-FTY and 10 μM SEW for 4 h. As shown in FIG. 18A-B, treatment of MG132 alone did not affect the expression of S1PR1. However, MG132 (MG in the figure) significantly inhibited 1R- or p-FTY induced S1PR1 degradation and restored S1PR1 expression from 66.3% to 94.2% for 1R and 51.8% to 80.3% for p-FTY. (S)-FTY720-phosphonate (15 in the figure) also caused slight degradation of S1PR1 expression (89.5%); however, MG132 restored (S)-FTY720-phosphonate-degraded S1PR1 expression to 99.2%.

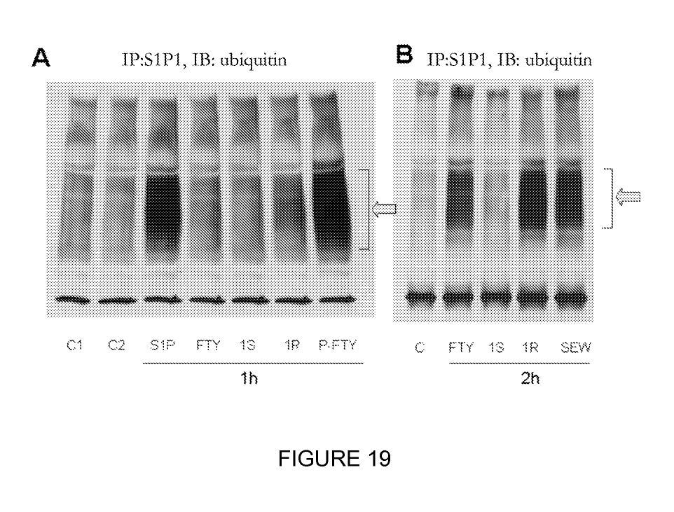

To measure the degree of ubiquitination of S1PR1 in response to treatment with S1PR1 agonists, HPAEC were treated with 1 μM S1P, FTY720 (FTY in the figure), (S)-FTY720-phosphonate (1S in the figure), 1R or phospho-FTY for 1 h. S1PR1 was immunoprecipitated by S1PR1Ab, and ubiquitination of S1PR1 was detected by ubiquitin Ab. As shown in FIG. 19A, treatment with 1 μM S1P and p-FTY at 1 h induced significant ubiquitination of S1PR1. However, 1 μM FTY720, 1S, or 1R at 1 h induced only slight ubiquitination of S1PR1. Next, HPAEC were treated with 1 μM FTY720, 1S, 1R or 10 μM SEW2871 for 2 h and ubiquitination of S1PR1 was detected. As shown in FIG. 19B, 1 μM FTY720, 1R or 10 μM SEW2871 for 2 h but not 15 induced significant ubiquitination of S1PR1. In conclusion, (S)-FTY720-phosphonate does not induce ubiquitination of S1PR1 in both 1 h and 2 h although all other S1PR1 agonists we tested induced ubiquitination of S1PR1 either at 1 h or 2 h.

Overall, these results demonstrate that (S)-FTY720-phosphonate (15) does not degrade S1PR1 protein expression as much as other agonists, that proteasome inhibition blocks the degradation of S1PR1 induced by 1R and p-FTY720., and that (S)-FTY720-phosphonate does not induce ubiquitination of S1PR1.

### Example 13

### Activation of Beta-Arrestin by S1P Analogs

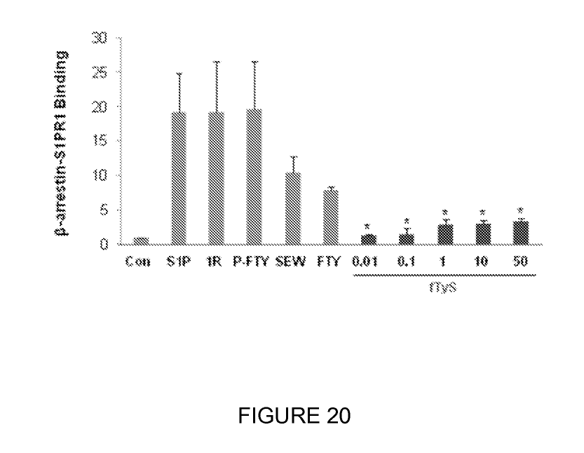

β-arrestin recruitment functions as a critical mechanism for damping down signaling following GPCR activation. The activation of 3-arrestin by S1P, FTY720, (S)-FTY720-phosphonate (1S), 1R, p-FTY and SEW2871 was examined by using Tango EDG1-bla U2OS Cell-based Assay (Invitrogen). Quiescent Tango™ EDG 1-bla U2OS cells were challenged by 1 μM (final concentration) S1P, (S)-FTY720-phosphonate, 1R, FTY720, p-FTY720 and 10 μM (final concentration) SEW2871 for 5 h. After another 2 h-incubation with fluorescence substrate, the fluorescence intensity (blue and green channels) were detected and the blue/green emission ratio was used as the activation indicator. As shown in FIG. 20, 1 μM S1P, 1R, FTY720, p-FTY720 and 10 μM SEW2871 strongly activated β-arrestin. However, (S)-FTY720-phosphonate, at concentrations of 1, 10, 50 μM, only slightly activated β-arrestin. Furthermore, the activation of β-arrestin was not escalated by increase of (S)-FTY720-phosphonate concentration. These data suggest that the activation of β-arrestin is critical for S1PR1 internalization and subsequent degradation. Thus, downstream S1PR1 signaling seems to differ in important ways following (S)-FTY720-phosphonate stimulation than with other agonists.

### Example 14

### Effects of S1P Analogs in a Murine Model of Bleomycin-Induced ALI

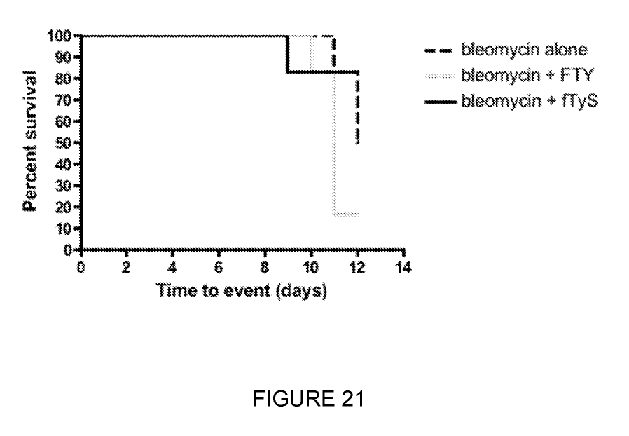

A bleomycin model of acute lung injury (ALI) was used to induce sustained lung inflammation in order to assess the potential use of (S)-FTY720-phosphonate for prolonged therapy (days to weeks). Bleomycin-injured mice receiving prolonged exposure to FTY720 exhibited increased lung injury and mortality (Shea et al., 2010, Am J Respir Cell Mol. Biol. 43(6): 662-73). C57/BL6 mice received bleomycin (1.2 U/kg) or sterile saline administered i.t. (intratracheally) and were then treated with FTY720, (S)-FTY720-phosphonate, (0.1 mg/kg i.p.) or PBS vehicle 3× a week until harvesting. Mice were followed for 14 days to assess mortality rates and multiple indices of lung injury. By day 12 following bleomycin instillation, only 17% of the FTY720-treated animals were still alive, whereas 83% of those receiving (S)-FTY720-phosphonate had survived (50% survival was observed in the bleomycin-only mice) (FIG. 21). Overall, mice receiving (S)-FTY720-phosphonate have a survival advantage over FTY720-treated animals in the bleomycin model of ALI.

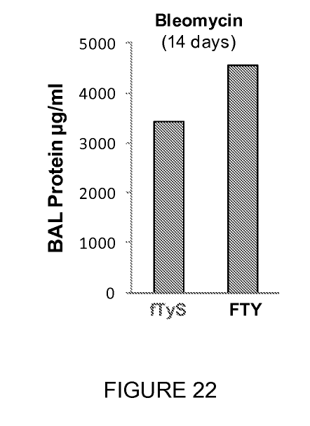

Decreased BAL protein was detected after 14 days in bleomycin-injured mice receiving (S)-FTY720-phosphonate compared to FTY720 (FIG. 22), suggesting reduced permeability after (S)-FTY720-phosphonate in this prolonged model of ALI.

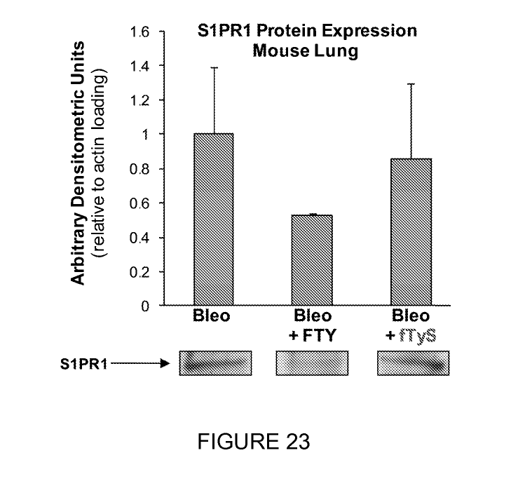

As discussed above, (S)-FTY720-phosphonate maintains S1PR1 expression in mouse lungs after 24 hr relative to other S1PR1 agonists (FIG. 17). The data shown in FIG. 23 extend these observations to 14 days. Mice receiving (S)-FTY720-phosphonate expressed higher levels of S1PR1 in their lungs compared to FTY720-treated animals after bleomycin. These data suggested that an important therapeutic advantage of (S)-FTY720-phosphonate is its ability to maintain S1PR1 protein levels in vivo over the prolonged period of time required to treat clinical ALI.

### Example 15

### Differential Effects of FTY720 Analogs on Endothelial Cell Barrier Function In Vitro

The effects of the (R)- and (S)-enantiomers of three FTY720 analogs (1=phosphonate, 2=enephosphonate, and 3=regioisomer) (see FIG. 24 for the structures of the analogs; note that 1S is (S)-FTY720-phosphonate) on vascular endothelial cell (EC) barrier integrity were measured by transendothelial electrical resistance (TER), a highly sensitive in vitro metric of permeability.

Human pulmonary artery endothelial cells (HPAEC) were obtained from Lonza Walkersville, Inc. (Walkersville, Md.) and were cultured in the manufacturer's recommended endothelial growth medium-2 (EGM-2) (Dudek et al. (2004), J Biol Chem 279: 24692-24700). Cells were grown at 37° C. in a 5% CO₂ incubator, and passages 6 to 9 were used for experiments. Media were changed 1 day before experimentation.

EC were grown to confluence in polycarbonate wells containing evaporated gold microelectrodes, and TER measurements were performed using an electrical cell-substrate impedance sensing system (Applied Biophysics, Troy, N.Y.) as follows (see also Garcia et al. (2001), J Clin Invest 108: 689-701). Endothelial cells were grown to confluence in polycarbonate wells containing evaporated gold microelectrodes (surface area, 10⁻³ cm²) in series with a large gold counter electrode (1 cm²) connected to a phase-sensitive lock-in amplifier. Current was applied across the electrodes by a 4,000-Hz AC voltage source with amplitude of 1 V in series with a 1 MΩ resistance to approximate a constant current source (˜1 μA). The in-phase and out-of-phase voltages between the electrodes were monitored in real time with the lock-in amplifier and subsequently converted to scalar measurements of transendothelial impedance, of which resistance was the primary focus. TER was monitored for 30 minutes to establish a baseline resistance (R₀) which, for bovine pulmonary endothelium, was typically between 8 to 12×10³Ω (wells with R₀<7×10³Ω were rejected). As cells adhere and spread out on the microelectrode, TER increases (maximal at confluence), whereas cell retraction, rounding, or loss of adhesion is reflected by a decrease in TER. These measurements provide a highly sensitive biophysical assay that indicates the state of cell shape and focal adhesion. Values from each microelectrode were pooled at discrete time points and plotted versus time as the mean±SE of the mean. TER values from each microelectrode were pooled as discrete time points and plotted versus time as the mean±S.E.M.

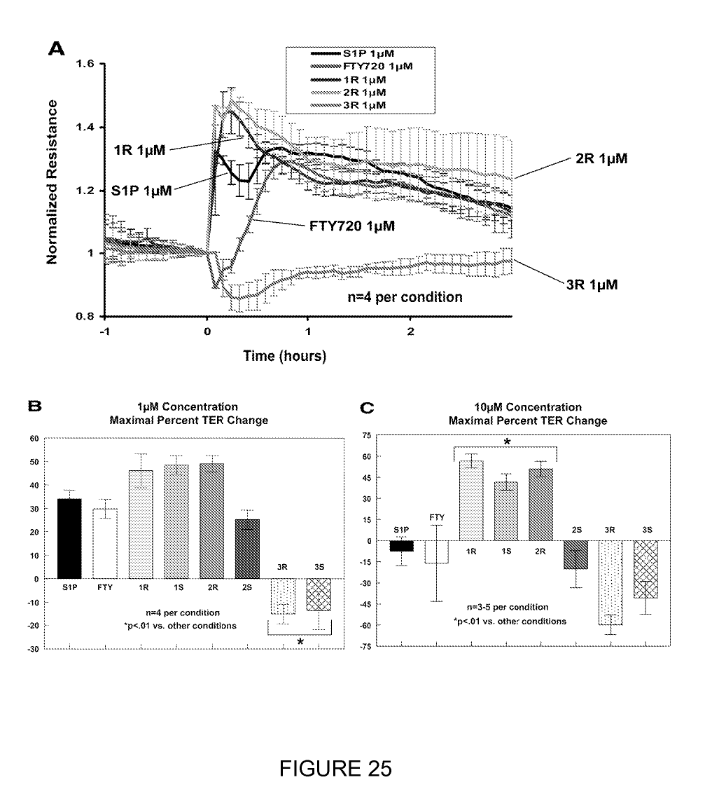

The (R)- and (S)-enantiomers of 1 and 2 are similar to S1P in that they produce rapid and sustained increases in TER (indicative of enhanced EC barrier function), whereas FTY720 itself induced a delayed onset of barrier enhancement (Dudek et al. (2007), Cell Signal 19: 1754-1764) that was slower to rise in TER relative to S1P and the FTY720 analogs (FIG. 25A; note that only (R)-enantiomer TER data are shown. (S)-Enantiomer results are similar and, therefore, not shown for simplicity). Interestingly, the FTY720 regioisomers 3R and 3S (in which the positions of the amino groups and one of the hydroxymethyl groups are interchanged) were barrier-disruptive at similar concentrations despite being structurally very similar to the parent FTY720 compound, indicating the sensitivity of this response to minor structural alterations. Although similar to S1P in the rapid induction of increased TER, the barrier-enhancing FTY720 analogs 1R, 1S, and 2R have a greater maximal percentage TER change at 1 μM compared with both S1P and FTY720 (FIG. 25B). Moreover, when the concentration of these compounds is increased to 10 μM, analogs 1R, 1S, and 2R exhibit even greater maximal TER elevation, whereas S1P, FTY720, and 2S are now somewhat barrier-disruptive at this dose (FIG. 25C), indicating that the barrier-enhancing effects of analogs 1R, 1S, and 2R are sustained over a wider concentration range than those of either S1P or FTY720. In fact, dose-response titrations of 1S, 1R, and 2R demonstrate that these analogs retain near maximal barrier-promoting effects over a range from 1 to 50 μM, suggesting a potential broader therapeutic index for these compounds compared with S1P or FTY720 (data not shown). The results also highlight the importance of enantiomer-specific effects as the enephosphonate analogs (2R and 2S) have diametrically opposing effects on EC barrier function at higher concentrations (≧10 μM).

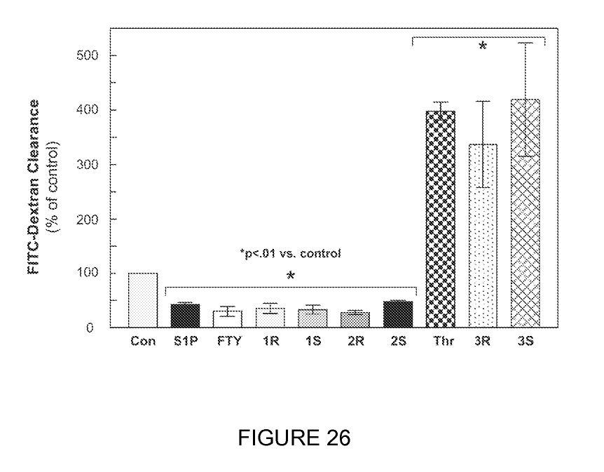

As a complementary approach to further characterize the barrier-protective effects of these FTY720 analogs in vitro, the permeability of FITC-labeled dextran across the pulmonary EC monolayer was assayed in response to treatment by the FTY720 analogs. Vascular permeability was tested using a transendothelial permeability assay, which was performed using labeled tracer flux across confluent EC grown on confluent polycarbonate filters (Vascular Permeability Assay Kit; Millipore Corporation) (Garcia et al. (1986), J Cell Physiol 128: 96-104). HPAEC plated on Transwell inserts were stimulated with S1P, FTY720, 1R, 1S, 2R, 2S (each at 1 μM), thrombin (1 unit/ml), 3R, or 3S (both 25 μM; lower concentrations did not alter permeability) for 1 h before addition of FITC-dextran. After a 2-h incubation, FITC-dextran clearance relative fluorescence was measured by excitation at 485 nm and emission at 530 nm. Data were normalized to unstimulated control. n=3 independent experiments per condition; *, p<0.01 versus unstimulated condition (see FIG. 26).

Whereas TER measurements are an assessment of EC permeability in terms of resistance to an electrical current, this assay allows for characterization of changes in EC permeability to higher molecular weight molecules. Compared with control EC, those treated with S1P, FTY720, or FTY720 analogs 1 and 2 all demonstrated significantly decreased permeability in this assay (FIG. 26), consistent with the TER data shown in FIG. 25. In contrast, the regioisomers (3R and 3S) increase EC permeability to a degree similar to thrombin, a well described and potent barrier-disrupting agent (Dudek and Garcia (2001), J Appl Physiol 91: 1487-1500).

### Example 16

### Differential Cytoskeletal Rearrangement and Intracellular Signaling of FTY720 Analogs

Immunofluorescence was used to assess cytoskeletal rearrangements of epithelial cells in response to treatment with FTY720 analogs. Confluent HPAEC were stimulated with vehicle control or 1 μM S1P, 1R, 2R, or 3R for 5 min or with FTY720 (1 μM) for 30 min. EC were then fixed in 3.7% formaldehyde for 10 min, permeabilized with 0.25% Triton X-100 for 5 min, washed in PBS, blocked with 2% bovine serum albumin in Tris-buffered saline with Tween 20 for 1 h, and then incubated for 1 h at room temperature with the primary antibody of interest. After washing, EC were incubated with the appropriate secondary antibody conjugated to immunofluorescent dyes (or Texas Red-conjugated phalloidin for actin staining) for 1 h at room temperature. After further washing with Tris-buffered saline with Tween 20, coverslips were mounted using Prolong Anti-Fade Reagent (Invitrogen) and analyzed using a Nikon Eclipse TE2000 inverted microscope (Nikon, Melville, N.Y.).

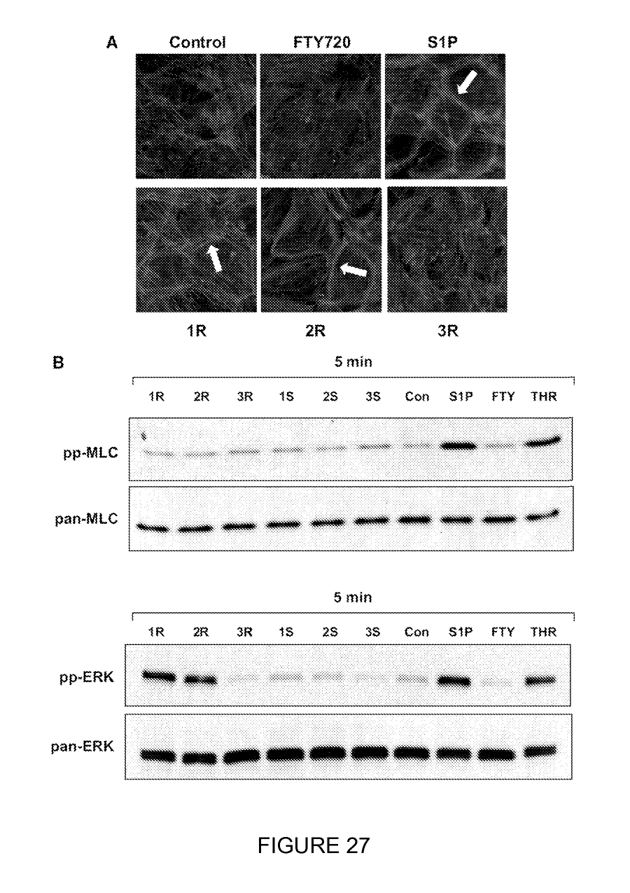

S1P generates dramatic EC cytoskeletal rearrangements such as enhanced cortical actin accumulation and peripheral MLC phosphorylation (Garcia et al. (2001), J Clin Invest 108: 689-701), which are not observed during FTY720-induced barrier enhancement (Dudek et al. (2007), Cell Signal 19: 1754-1764). Because the barrier enhancing analogs 1 and 2 produce immediate TER elevation similar to S1P (FIG. 25A), next evaluated was whether these compounds elicited rapid F-actin cytoskeletal rearrangements similar to exposure to S1P (FIG. 27A) Immunofluorescent analysis reveals that compounds 1 and 2 rapidly induced (within 5 min) increased cortical actin ring formation in the periphery of pulmonary EC characteristic of S1P-induced barrier enhancement (FIG. 27A, arrows) (Garcia et al., 2001, J Clin Invest 108: 689-701). In contrast, FTY720 failed to elicit cortical actin ring formation early at 5 min (data not shown) or at data time points (30 min) associated with peak TER elevation (FIG. 27A). Interestingly, the barrier-disrupting FTY720 analog 3 did not produce dramatic F-actin rearrangements.

After treatment as outlined for individual experiments, confluent HPAEC were lysed for Western blotting with phospho-MLC, pan-MLC, phospho-ERK, or pan-ERK antibodies. Sample proteins were separated with 4 to 15% SDS-PAGE gels (Bio-Rad, Hercules, Calif.) and transferred onto Immobilon-P polyvinylidene difluoride membranes (Millipore Corporation). Membranes were then immunoblotted with primary antibodies (1:500-1000, 4° C., overnight) followed by secondary antibodies conjugated to horseradish peroxidase (1:5000, room temperature, 30 min) and detected with enhanced chemiluminescence (Pierce ECL or SuperSignal West Dura; Pierce Biotechnology, Rockford, Ill.) on Biomax MR film (Carestream Health, Rochester, N.Y.).

Whereas the barrier-enhancing FTY720 analogs exhibited similarities to S1P in cortical actin ring formation, their effects on intracellular signaling events were varied (FIG. 27B). Evaluation of EC lysates for MLC and ERK phosphorylation demonstrated increased MLC and ERK phosphorylation at 5 min in response to SIP, whereas analogs 1R and 2R caused increased phosphorylation of ERK at 5 min. Neither FTY720 nor any of its analogs induced significant MLC phosphorylation over this time frame. Interestingly, the enantiomers 1S and 2S differed from 1R and 2R in terms of ERK signaling because the former failed to induce phosphorylation of this kinase. Thus, these closely related compounds were not equivalent in terms of their downstream signaling effects on cultured pulmonary EC. The barrier-disruptive FTY720 regioisomers 3R and 3S did not increase ERK or MLC phosphorylation (5 min), unlike the well described barrier-disruptive agent thrombin (Dudek and Garcia, 2001, J Appl Physiol 91: 1487-1500).

### Example 17

### Responses in Intracellular Calcium Levels to FTY720 Analogs

To further explore the mechanistic differences in barrier regulation, intracellular calcium responses to the FTY720 analogs, S1P, and FTY720 were examined using Fura-2 (Harbeck et al., 2006, Sci STKE 2006: p16). HPAEC plated on 25-mm glass coverslips were loaded with 1 μM Fura-2/acetoxymethyl ester (Invitrogen, Carlsbad, Calif.) for 20 min at 37° C. in KRBH5 buffer (Krebs-Ringer-bicarbonate solution containing 119 mM NaCl, 4.7 mM KCl, 2.5 mM CaCl₂, 1 mM MgCl₂, 1 mM KH₂PO₄, 25 mM NaHCO₃, mM HEPES-NaOH (pH 7.4), and 5 mM glucose). After replacement of the Fura-2 loading buffer with fresh KRBH5, coverslips were placed into the specimen stage of an inverted fluorescence microscope (Nikon TE-2000U). A Nikon Super Fluor 10× objective was used for these studies. Filters (340- and 380-nm excitation and 530-nm emission) were used for Fura-2 dual excitation ratio imaging. Imaging data acquisition and analysis were accomplished using MetaMorph/MetaFluor software (Molecular Devices, Sunnyvale, Calif.) and OriginPro 7E (OriginLab Corp, Northampton, Mass.). Fura-2 340/380 dual excitation ratios were converted to [Ca²⁺] by in situ calibration. To calibrate Fura-2 ratios, Rₘₐₓ was obtained by treating cells with 10 μM ionomycin and 2.5 mM Ca²⁺, and Rₘᵢₙ was obtained by treating cells with EGTA to a final concentration of 10 mM. Fura-2 ratios were converted to [Ca²⁺] using the equation: [Ca²⁺]=(Kd′[(R−Rₘᵢₙ)/(Rₘₐₓ−R)]×S_{f}/S_{b}) (Grynkiewicz et al., 1985, J Biol Chem 260: 3440-3450), where Kd′ is the dissociation constant for Fura-2 in the cytosol (225 nM) and S_{f} and S_{b} are the measured emission intensities at 380 nm for Ca²⁺-free and Ca²⁺-bound Fura-2, respectively. Data summaries for all Ca²⁺ measurements are expressed as the means±S.E.

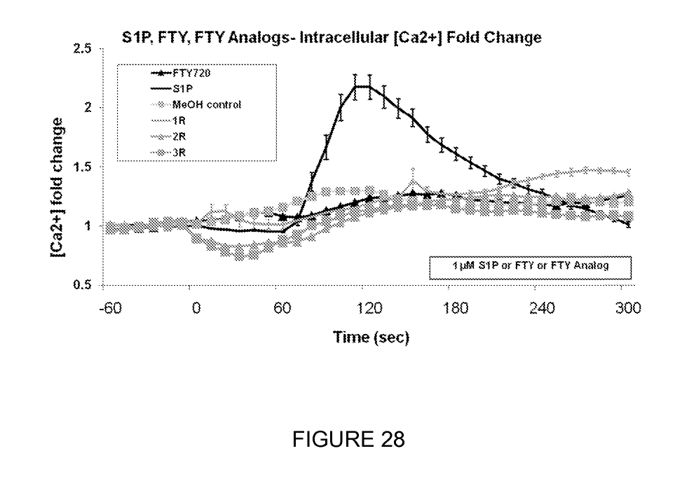

Previous studies have described a brief but substantial increase in intracellular calcium (Ca²⁺) following S1P exposure in pulmonary endothelial cells (Garcia et al., 2001, J Clin Invest 108: 689-701), whereas FTY720 failed to increase intracellular Ca²⁺ (Dudek et al., 2007, Cell Signal 19: 1754-1764). Changes in HPAEC [Ca²⁺]ᵢ after treatment with FTY720 analogs, S1P, FTY720, and vehicle (all at 1 μM concentration) revealed that only S1P produced a transient Ca²⁺ spike (FIG. 28), demonstrating that the FTY720 analog-induced barrier enhancement does not require the calcium signaling observed in association with S1P.

### Example 18

### Mechanistic Components of FTY720 Analog-Induced Barrier Enhancement

Similar to S1P and FTY720 (Dudek et al., 2007, Cell Signal 19: 1754-1764), TER elevation induced by all four barrier-enhancing compounds (1R, 1S, 2R, and 2S) was significantly inhibited by preincubation with either pertussis toxin (PTX) or genistein (EMD Biosciences, San Diego, Calif.), a nonspecific tyrosine kinase inhibitor (Table 7), indicating essential involvement of Gᵢ-coupled signaling and tyrosine phosphorylation events in these barrier-enhancing responses. In this experiment, confluent HPAEC were plated on gold microelectrodes and then stimulated with 1 μM S1P, FTY720, 1R, 1S, 2R, or 2S after either a 2-h preincubation with 100 ng/ml PTX, 30-min preincubation with 200 μM genistein (Gen), or 2-h preincubation with 2 mM MβCD or their respective vehicle controls. Data were pooled from multiple TER experiments (4-10 independent experiments per condition) and expressed as percentage inhibition of maximal barrier enhancement at 60 min relative to agonist-only control. Signaling pathways initiated in membrane lipid rafts were essential to S1P- and FTY720-induced barrier enhancement (Singleton et al., 2005, FASEB J 19: 1646-1656; Dudek et al., 2007, Cell Signal 19: 1754-1764). Consistent with the involvement of lipid rafts in FTY720 analog barrier enhancement, the lipid raft-disrupting agent, methyl-β-cyclodextrin (MβCD), significantly attenuated their TER elevation (Table 7). Overall, these in vitro data supported a barrier-enhancing pathway induced by FTY720 analogs 1R, 15, 2R, and 2S that probably included lipid raft signaling and Gᵢ-linked receptor coupling to downstream tyrosine phosphorylation events.

TABLE 7
Pharmacologic inhibitor effects on
FTY720 analog barrier enhancement

| | % Inhibition of Maximal TER Response (of Agonist-Only Control) | | |
|---|---|---|---|
| | PTX** | Gen* | MβCD** |
| S1P | 98.35 (±0.25) | 42.4 (±13.9) | 81.5 (±8.0) |
| FTY | 84.0 (±9.1) | 86.2 (±10.7) | 88.1 (±5.6) |
| 1R | 79.2 (±5.9) | 54.3 (±14.7) | 67.0 (±19.7) |
| 1S | 92.8 (±2.6) | 51.9 (±21.0) | 97.2 (±0.8) |
| 2R | 88.1 (±7.8) | 41.1 (±12.2) | 87.4 (±5.4) |
| 2S | 76.3 (±12.0) | 91.6 (±2.4) | 95.2 (±1.0) |

**All EC treated with this inhibitor exhibit P < 0.01 decreased TER compared with agonist-only control.
*All EC treated with this inhibitor exhibit P < 0.05 decreased TER compared with agonist-only control.

### Example 19

### Protective Effects of (S)-FTY720-Phosphonate in an LPS-Induced Murine Lung Injury Model

To extend these in vitro findings that FTY720 analogs promoted lung EC integrity, a murine model of LPS-induced lung injury was used to examine the in vivo effects of these compounds on pulmonary vascular leak and inflammatory injury. Preliminary studies indicated that 1S was superior to the other barrier-promoting analogs (1R, 2R, and 2S) in this model (data not shown). Therefore, 1S was further characterized with regard to pulmonary vascular leak and inflammatory injury in this mouse model. Intratracheal administration of LPS (2.5 mg/kg) produced significant murine inflammatory lung injury at 18 h as assessed by measurements of BAL total protein and cell count, BAL albumin, and lung tissue albumin (Peng et al., 2004, Am J Respir Crit. Care Med 169: 1245-1251). Moreover, LPS increased tissue MPO activity, another reflection of lung parenchymal phagocyte infiltration, compared with control mice (Peng et al., 2004, Id.).

All experiments and animal care procedures were approved by the Chicago University Animal Resource Center and were handled according to the Animal Care and Use Committee Guidelines at the University of Chicago. C57BL/6 (20-25 g) mice were purchased from The Jackson Laboratory (Bar Harbor, Me.). Mice were housed with access to food and water in a temperature-controlled room with a 12-h dark/light cycle. For experiments performed in intact animals, male C57BL/6 mice (8-10 weeks) were anesthetized with intraperitoneal ketamine and acetylpromazine mixture according to the approved protocol. Escherichia coli LPS solution (2.5 mg/kg) or sterile saline was instilled intratracheally via a 20-gauge catheter. Simultaneously, mice received either FTY720 or analogs (in doses: 0.01, 0.1, 0.5, 1, and 5 mg/kg i.p.) or PBS as vehicle. The animals were allowed to recover for 18 h. BAL and lungs were collected and stored at −70° C. for evaluation of lung injury.

Pure BAL fluids prepared for protein measurement or myeloperoxidase activity (MPO) lung homogenates were used to test albumin concentration. The assay was performed in 96-well plastic plates (Nalge Nunc A/S, Roskilde, Denmark). Plates were coated with mouse albumin (Bethyl Lab, Montgomery, Tex.), washed, and blocked. Aliquots (100-μl) of the sample or standard and 100 μl of goat anti-mouse albumin antibody (horseradish peroxidase-conjugated) (1:50,000) were then added, followed by incubation at 37° C. for 1 h. Finally, the substrate 3,3′,5,5′-tetramethylbenzidine was added for 10 min, and the reaction stopped by adding 100 μl of 2 M H₂SO₄. The absorbance at 450 nm was read on a kinetic microplate reader (Molecular Devices).

Myeloperoxidase (MPO) was isolated and measured from snap-frozen right lungs as follows (see also Remick et al., 1990, Am J Pathol 136: 49-60). The right lung was homogenized in 1 ml of 50 mM potassium phosphate, pH 6.0, with 0.5% hexadecyltrimethylammonium bromide. The resulting homogenate was sonicated and then centrifuged at 12,000 g for 15 min. The supernatant was mixed 1:30 with assay buffer (100 mM potassium phosphate, pH 6.0, 0.005% H₂O₂, 0.168 mg/ml o-dianisidine hydrochloride), and absorbance read at 490 nm. MPO units were calculated as the change in absorbance with respect to time.

Peripheral blood was examined by the Missouri University Research Animal Diagnostic Laboratory (Columbia, Mo.) for determination of total blood cell counts and differentials in blood samples.

Values are shown as the mean±S.E. Data were analyzed using a standard Student's t test or one-way analysis of variance, groups were compared by Newman-Keuls test, and significance in all cases was defined at p<0.05.

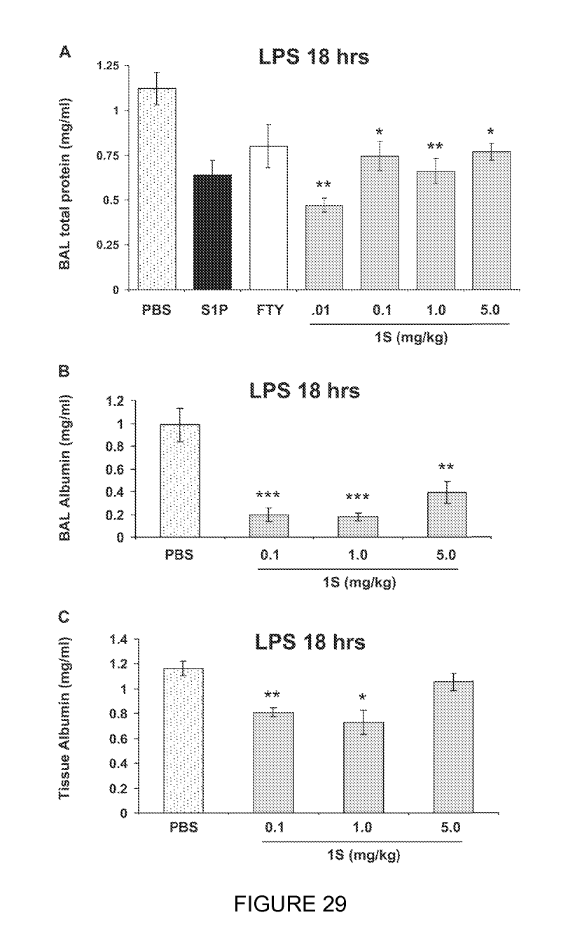

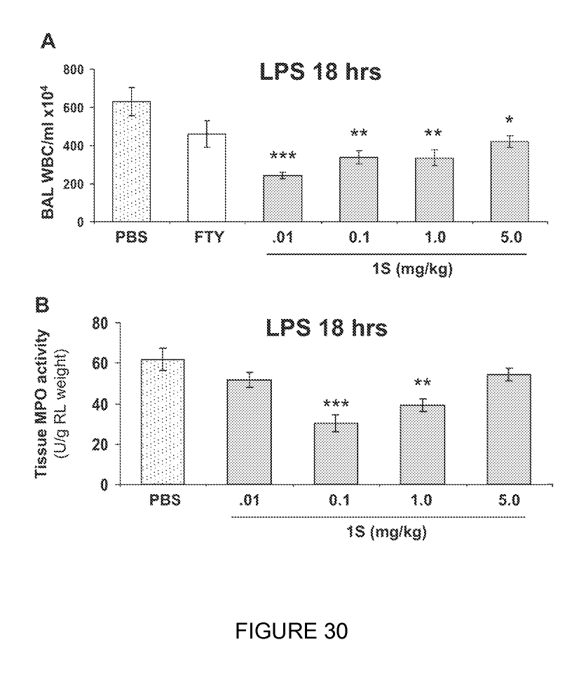

Intraperitoneal injection of a single dose of FTY720 analog 1S (0.1-5.0 mg/kg) delivered 1 h after LPS exposure significantly reduced capillary leak relative to PBS control at all of the concentrations studied as measured by total BAL protein concentrations (FIG. 29A). This reduction in permeability by 1S was comparable to that achieved by S1P or FTY720. In addition, 15 significantly reduced LPS-induced albumin leakage from the vascular space into both the surrounding lung tissue and BAL (FIGS. 29B and 29C), as well as BAL WBC accumulation and lung tissue MPO activity (FIGS. 30A and B). These combined data suggested that the optimal protective dose of 15 is 0.1 to 1.0 mg/kg in this model.

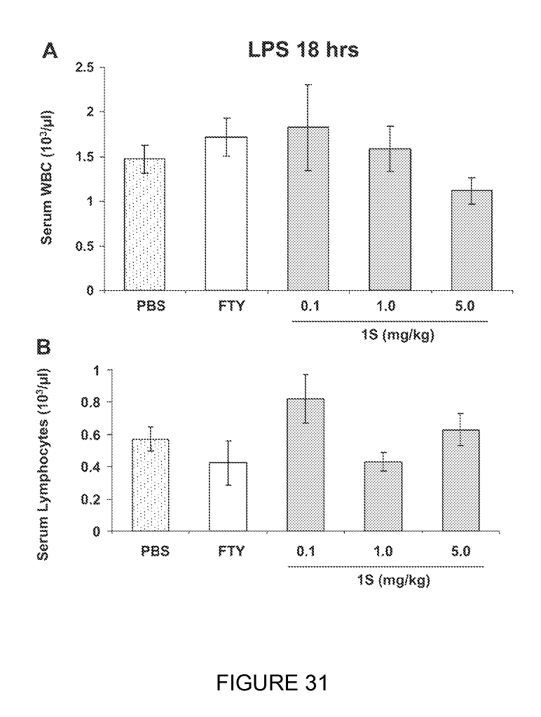

One potential concern when using FTY720 or related compounds in sepsis-related processes such as acute lung injury is the known lymphopenia effect of the parent compound (Kovarik et al., 2004, Ther Drug Monit 26: 585-587). Therefore, peripheral blood WBC levels were assessed in this mouse model. For comparison, at baseline in control mice (no LPS), total circulating WBC is 4.11±1.58×10³/μl and the lymphocyte count is 3.57±1.74×10³/μl (n=6), so these levels are significantly suppressed (p<0.001 for both total WBC and lymphocyte count) by LPS alone in this model 18 h after its administration (FIG. 31). However, 1S treatment in these mice does not further alter peripheral blood leukocyte and lymphocyte levels relative to PBS controls (FIG. 31), suggesting that the 15 analog does not produce additional immunosuppression in this LPS model. Interestingly, FTY720 itself also does not suppress circulating WBC levels relative to PBS controls in this model of inflammatory lung injury. In summary, the FTY720 analog 15 decreased multiple indices of LPS-induced pulmonary injury in this murine model without apparent hematologic toxicity. In summary, using multidimensional approaches a murine model of RILI which exhibits temporal increases in lung permeability, leukocyte influx, and pro-inflammatory cytokine secretion, was established and validates, having findings compatible with the limited reports of human and murine models of thoracic irradiation (Williams et al., 2004, Radiat Res 161:560-567). Using this model, profound clinical promise of simvastatin as a protective strategy to attenuate the untoward effects of RILI was identified, and suggested that simvastatin can be used as a novel alternative to aggressive corticosteroid therapy in RILI. In view of the availability, affordability, and favorable safety profile of this class of drugs, simvastatin-like drugs may potentially allow for radiation dose escalation while enhancing outcomes of patients receiving radiotherapy for thoracic malignancies.

In addition, these results provided evidence of significant protection conferred by (S)-FTY720-phosphonate and, to a lesser extent, SEW 2871 (albeit surprisingly showing minimal efficacy of FTY720). Protection against RILI by specific S1P analogs offered strong evidence in support of the use of these novel agonists in relevant patient populations exposed to thoracic radiation. Furthermore, these data suggested that sphingolipid components can be used as novel RILI biomarkers, and targets for novel and effective therapeutic strategies in RILI.

Studies using a bleomycin-induced mouse model of ALI demonstrate the therapeutic effectiveness of (S)-FTY720-phosphonate in an additional model of lung injury.

Lastly, results in a murine model of LPS-induced acute lung injury provide important mechanistic insights into the regulation of EC barrier function and demonstrate the potential therapeutic utility of several novel FTY720 analogs to reverse the pulmonary vascular leak that characterizes ALI. (S)-FTY720-phosphonate is particularly promising in the LPS-induced model both in vitro and in vivo. Moreover, animal data suggest that, at doses sufficient to protect against lung injury, FTY720 and its derivative (S)-FTY720-phosphonate (1S) do not adversely affect circulating WBC levels during LPS-induced inflammatory states and thus may be appropriate to use in critically ill patients with infection-associated ALI.

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

We claim:

1. A method of treating or reducing the risk of developing radiation-induced acute lung injury (RILI) in a mammal comprising the step of administering to a mammal in need thereof an effective amount of an FTY720 derivative or analog or SEW 2871.

2. The method of claim 1, wherein the FTY720 derivative or analog or SEW 2871 is administered before radiation.

3. The method of claim 1, wherein the FTY720 derivative or analog or SEW 2871 is administered after radiation.

4. The method of claim 1, wherein the FTY720 derivative or analog or SEW2871 is administered concurrently with radiation.

5. The method of claim 1, wherein the mammal is subjected to thoracic radiation therapy.

6. The method of claim 5, wherein the FTY720 derivative or analog reduces weight loss or hair loss associated with the radiation therapy.

7. The method of claim 6 wherein the FTY720 derivative or analog is administered before the radiation therapy.

8. The method of claim 6, wherein the FTY720 derivative or analog is administered after the radiation therapy.

9. The method of claim 6, wherein the FTY720 derivative or analog is administered concurrently with the radiation therapy.

10. A method of reducing weight loss or hair loss associated with thoracic radiation therapy in a mammal comprising the step of administering to a mammal in need thereof an effective amount of an FTY720 derivative or analog to reduce weight loss or hair loss.

11. The method of claim 10 wherein the FTY720 derivative or analog is administered before the radiation therapy.

12. The method of claim 10, wherein the FTY720 derivative or analog is administered after the radiation therapy.

13. The method of claim 10, wherein the FTY720 derivative or analog is administered concurrently with the radiation therapy.

14. A method of treating or reducing the risk of developing acute lung injury in a mammal comprising the step of administering to a mammal in need thereof an effective amount of an FTY720 derivative or analog or SEW 2871.

15. The method of claim 14, wherein the acute lung injury is endotoxin-induced lung injury.

16. The method of claim 15, wherein the endotoxin is lipopolysaccharide (LPS).

17. The method of claim 1 or 14, wherein the administration of an FTY720 derivative or analog or SEW 2871 reduces vascular leakage or vascular permeability in the mammal, wherein vascular leakage or vascular permeability occurs as a result of acute lung injury.

18. The method of claim 1 or 14, wherein the administration of an FTY720 derivative or analog or SEW 2871 reduces BAL protein levels in the mammal, wherein the BAL protein levels increase as a result of acute lung injury.

19. The method of claim 1 or 14, wherein the administration of an FTY720 derivative or analog or SEW 2871 reduces BAL cell count in the mammal, wherein BAL cell count increases as a result of acute lung injury.

20. The method of claim 1 or 14, wherein the administration of an FTY720 derivative or analog or SEW 2871 increases alveolar cell integrity or increases endothelial cell integrity in the mammal, wherein alveolar cell integrity or endothelial cell integrity decreases as a result of acute lung injury.

21. The method of claim 1 or 14, wherein the administration of an FTY720 derivative or analog or SEW 2871 reduces lung inflammation in the mammal, wherein lung inflammation occurs as a result of acute lung injury.

22. The method of claim 1 or 14, wherein the administration of an FTY720 derivative or analog or SEW 2871 reduces dysregulation of the ceramide/sphingolipid metabolic pathway in the lung of the mammal, wherein the dysregulation of the ceramide/sphingolipid metabolic pathway in the lung occurs as a result of acute lung injury.

23. The method of claim 1 or 22, wherein the dysregulation of the ceramide/sphingolipid metabolic pathway in the lung is indicated by decreased combined levels of sphingosine 1 phosphate (S1P) and dihydro-S1P (DHS1P) in a sample from the lung.

24. The method of claim 22, wherein the dysregulation of the ceramide/sphingolipid metabolic pathway in the lung is indicated by increased levels of ceramide in a sample from the lung.

25. The method of claim 23 wherein the sample from the lung is a lung tissue sample, a BAL fluid sample, or a plasma sample.

26. The method of claim 24 wherein the sample from the lung is a lung tissue sample, a BAL fluid sample, or a plasma sample.

27. The method of claim 25, wherein the sample is a BAL fluid sample.

28. The method of claim 26, wherein the sample is a BAL fluid sample.

29. The method of any one of claims 1-28, wherein the FTY720 analog or derivative is the (R)- or (S)-enantiomer of FTY720 phosphonate, the (R)- or (S)-enantiomer of FTY720-enephosphonate, or the (R)— or (S)-enantiomer of FTY720 regioisomer.

30. The method of claim 29, wherein the FTY720 analog or derivative is the (R)- or (S)-enantiomer of FTY720 phosphonate.

31. The method of claim 30, wherein the FTY720 analog or derivative is the (S)-enantiomer of FTY720 phosphonate (tysiponate).

32. The method of claim 29, wherein the mammal is a human.

33. The method of claim 31, wherein the mammal is a human.

34. A pharmaceutical dosage form comprising an FTY720 analog or derivative or SEW 2871 in an amount of about 0.7 mg/dosage unit-about 500 mg/dosage unit.

35. The pharmaceutical dosage form of claim 34, wherein the FTY720 analog or derivative or SEW 2871 is present in an amount from about 0.7 mg/dosage unit-about 70 mg/dosage unit.

36. The pharmaceutical dosage form of claim 34, wherein the FTY720 analog or derivative or SEW 2871 is present in an amount from about 70 mg/dosage unit-about 500 mg/dosage unit.

37. The pharmaceutical dosage form of any one of claims 34-36, wherein the FTY720 analog or derivative is the (R)- or (S)-enantiomer of FTY720 phosphonate, the (R)- or (S)-enantiomer of FTY720-enephosphonate, or the (R)- or (S)-enantiomer of FTY720 regioisomer.

38. The pharmaceutical dosage form of claim 37, wherein the FTY720 analog or derivative is the (R)- or (S)-enantiomer of FTY720 phosphonate,

39. The pharmaceutical dosage form of claim 38, wherein the FTY720 analog or derivative is (S)-enantiomer of FTY720 phosphonate,

40. A method of diagnosing radiation-induced lung injury in a mammal comprising the step of assaying a sample from a mammal after exposure to radiation to detect levels of sphingosine 1 phosphate (S1P), dihydro S1P (DHS1P) or ceramide wherein lung injury is diagnosed when the combined levels of S1P and DHS1P are reduced in the sample from the mammal as compared to the combined levels of S1P and DHS1P in a sample from a control mammal or when the ceramide levels are increased in a sample from the mammal as compared to the ceramide levels in a sample from the control mammal.

41. The method of claim 40 wherein lung injury is diagnosed when the ceramide levels are increased in a sample from the mammal as compared to the ceramide levels in a sample from the control mammal.

42. The method of claim 40 or 41, wherein the sample is a lung tissue sample, a BAL fluid sample, or a plasma sample.

43. The method of claim 42, wherein the sample is a BAL fluid sample.

44. The method of claim 42, wherein the mammal is a human.

45. The method of claim 40 or 41, wherein the sample is taken from the mammal four-six weeks after exposure to radiation.

46. The method of claim 45, wherein the sample is taken from the mammal four weeks after exposure to radiation.

47. The method of claim 45, wherein the sample is taken from the mammal six weeks after exposure to radiation.

48. The method of claim 45, wherein the mammal is a human
